(19) United States
(12) Patent Application Publication
(10) Pub. No.: US 2022/0175269 A1
(43) Pub. Date: Jun. 9, 2022

(54) Methods and Systems for Body Lumen Medical Device Location

(71) Applicant: Frond Medical Inc., Fremont, CA (US)

(72) Inventors: Xin Lu, Palo Alto, CA (US); Rose Monaghan, Los Gatos, CA (US); Shihming Huang, Fremont, CA (US); Xi Lin, San Jose, CA (US); Raymond Chan, San Diego, CA (US); Homer Pien, Boston, MA (US)

(21) Appl. No.: 17/643,066

(22) Filed: Dec. 7, 2021

Related U.S. Application Data
(60) Provisional application No. 63/122,233, filed on Mar. 13, 2021.
(60) Provisional application No. 63/122,424, filed on Dec. 7, 2020.
(60) Provisional application No. 63/122,433, filed on Dec. 7, 2020.
(60) Provisional application No. 63/176,342, filed on Apr. 18, 2021.
(60) Provisional application No. 63/176,341, filed on Apr. 18, 2021.

(51) Int. Cl. A61B 5/06; A61B 5/00; A61B 90/00

(52) U.S. Cl. CPC A61B 5/066; A61B 5/6851; A61B 5/6852; A61B 5/6862; A61B 90/39; A61B 5/742; A61B 2090/364; A61B 2090/3966; A61B 5/0066; A61B 5/0095; A61B 2090/3937

(57) ABSTRACT

Systems and methods for locating a medical device in a body lumen are provided. A first flexible elongate instrument comprises a plurality of imaging markers, and a location information sensor is disposed at the first flexible elongate instrument or at a second flexible elongate instrument configured for relative movement with respect to the first flexible elongate instrument. A processor is configured to establish a reference coordinate system based on the plurality of imaging markers, which are visible in a medical image comprising the first flexible elongate instrument disposed in a body lumen, receive diagnostic scan or therapeutic delivery information at a plurality of locations of the body lumen from the first or second flexible elongate instrument, and correlate the information with the imaging markers. A display configured to display a composite image comprising the correlated diagnostic scan or therapeutic delivery information and the imaging markers.

### RELATED APPLICATIONS

This application claims the benefit of U.S. Provisional Application No. 63/122,233, filed on Dec. 7, 2020; U.S. Provisional Application No. 63/122,424, filed on Dec. 7, 2020; U.S. Provisional Application No. 63/122,433, filed on Dec. 7, 2020; U.S. Provisional Application No. 63/176,342, filed on Apr. 18, 2021; and U.S. Provisional Application No. 63/176,341, filed on Apr. 18, 2021. The entire teachings of the above applications are incorporated herein by reference.

### BACKGROUND

Intracoronary imaging is often used to accurately measure vessel and stenosis dimensions, assess vessel integrity, characterize lesion morphology and aide in body lumen procedures, including percutaneous coronary intervention (PCI) procedures. The frequency of complex percutaneous coronary interventions has steadily increased in recent years due to clinical benefits provided by the interventions, which can increase the life expectancy and quality of life for patients suffering from endovascular neurosurgical, cardiovascular, and peripheral artery diseases. Various diagnostic and therapeutic medical devices (e.g., guidewires, balloons, atherectomy, lithotripsy, stents, imaging and physiology diagnostic modalities, X-ray angiography, and fluoroscopy) enable radiologists, cardiologists, and vascular specialists to visualize a patient's intra-vasculature to guide treatment decisions and to perform intervention procedures. Often, X-ray fluoroscopy with contrast injection is used to guide physicians to position devices (e.g., stents, guidewires, and balloons) toward targeted lesion locations along a guidewire within the endo-vasculature.

In a PCI procedure, vascular access is typically gained through an arterial entry point, such as the radial, brachial, or femoral artery, or through a venous puncture. From the entry point, a physician can access the vasculature of organs such as heart, lungs, kidneys, and brain by advancing a guidewire into the patient until a distal end of the guidewire crosses, for example, a lesion to be treated. After the guidewire position is finalized and situated such that it is viewable on an angiographic image, a desired therapeutic and/or diagnostic device is mounted on a proximal end of the guidewire. The therapeutic and/or diagnostic device is then advanced towards the distal end to the feature of interest.

Depending upon the clinical situation, imaging and/or physiological probes, such as Intravascular Ultrasound (IVUS), Optical coherence tomography (OCT) and Fractional Flow Reserve (FFR) devices, can be used for pre-intervention assessment, such as for determining lesion location, lesion dimension, plaque morphology, and coronary pressure at an area of interest. Endoluminal diagnostic modalities, such as IVUS, OCT, and FFR, which are able to generate more detailed vessel lumen information than that which can be obtained from X-ray imaging alone, are widely used for minimally invasive PCI procedures.

Endoluminal device guidance generally requires a live display of the device's movement inside of a body lumen. The methods currently available for guidance and positioning are based on real-time X-ray angiographic imaging, such that both a blood vessel's lumen path and the device inside of the lumen are continuously visible during the procedure. X-ray imaging for blood vessel diagnosis and device guidance emits X-rays at many frames per second and often requires contrast fluid injection, which allows for visualization of the vessel to help clinicians locate and position medical instruments. This practice results in high radiation exposure to both patients and clinicians, as well as the delivery of large volumes of contrast agents to patients, which are harmful to the kidneys.

There exists a need for improved systems and methods for providing endoluminal device guidance and locating medical devices within a body lumen.

### SUMMARY

Systems and methods for locating a medical device in a body lumen are provided. Such systems and methods can advantageously provide for improved accuracy over existing positioning methods and reduced radiation exposure for clinicians and patients. The example systems and methods below are generally described within the example context of an intravascular diagnostic scan and radiopaque imaging markers; however, the methods and systems can be applied to other endoluminal applications and can make use of imaging markers visible in modalities other than X-ray.

A system for locating a medical device in a body lumen includes a first flexible elongate instrument comprising a plurality of imaging markers (e.g., radiopaque imaging markers) and a location information sensor disposed at the first flexible elongate instrument or at a second flexible elongate instrument configured for relative movement with respect to the first flexible elongate instrument (e.g., parallel, relative movement). The system further includes a processor configured to: establish a reference coordinate system based on the plurality of imaging markers, the plurality of imaging markers being visible in a medical image comprising the first flexible elongate instrument disposed in a body lumen, receive diagnostic scan or therapeutic delivery information at a plurality of locations of the body lumen from the first or second flexible elongate instrument, and correlate the diagnostic scan or therapeutic delivery information with the imaging markers for the plurality of locations based on the reference coordinate system and location information as sensed by the location information sensor. The system further includes a display configured to display a composite image comprising the correlated diagnostic scan or therapeutic delivery information and the imaging markers.

The processor can be further configured to receive the medical image (e.g., an X-ray image, such as an X-ray angiogram) comprising the first flexible elongate instrument disposed in the body lumen.

The location information sensor can be disposed on the first flexible elongate instrument. For example, the location information sensor can be a sensor, such as an optical sensor, configured to detect encoding markers of the second flexible elongate instrument. The first flexible elongate instrument can be a guidewire, and the second flexible elongate instrument can be or include the diagnostic or therapeutic device. The diagnostic device can be, for example, an intravascular ultrasound (IVUS) device, or an optical coherence tomography (OCT) device, a fractional flow reserve (FFR) catheter, a photoacoustic device, an endoscopic device, an arthroscopic device, or a biopsy device. The therapeutic device can be, for example, an angioplasty device, an embolization device, an ablation device, a drug-delivery device, an optical delivery device, an atherectomy device, or an aspiration device. The second flexible elongate instrument can include the encoding markers disposed at an inner circumferential surface of a catheter or liner configured for advancement over the first flexible elongate instrument.

The location information sensor can be disposed on the second flexible elongate instrument. For example, the location information sensor can be a sensor (e.g., an optical sensor) configured to detect encoding markers of the first flexible elongate instrument. The first flexible elongate instrument can be, for example, a fractional flow reserve (FFR) wire.

The location information sensor can be a diagnostic sensor disposed on the second flexible elongate instrument. For example, the first flexible elongate instrument can include a signal emitter configured to emit a signal for detection by the diagnostic sensor. The signal emitter can be an ultrasound transducer, an optical light emitter, or a signal reflector configured to reflect a signal originating from the diagnostic sensor. Correlating the diagnostic scan information with the imaging markers can include establishing a co-position location based on the detected signal.

The first flexible elongate instrument can be a diagnostic device, and the location information sensor can be a sensor that detects a push distance, a pullback distance, or a combination thereof of the diagnostic device. Correlating the diagnostic scan information with the imaging markers can include establishing a start location of a diagnostic sensor of the diagnostic device based on a relative position of the diagnostic sensor to at least one of the plurality of imaging markers.

The second flexible elongate instrument can be a diagnostic device comprising at least one imaging marker, and the location information sensor can be a sensor that detects a push distance, a pullback distance, or a combination thereof of the diagnostic device. Correlating the diagnostic scan information with the medical image can include establishing a start location of a diagnostic sensor of the diagnostic device based on a relative position of the at least one imaging marker of the diagnostic device and at least one of the plurality of imaging markers of the first flexible elongate instrument.

The system can include the second flexible elongate instrument. The location information sensor can be disposed at a distal portion of the first or second flexible elongate instrument. The reference coordinate system can be one-dimensional, two-dimensional, or three-dimensional. For example, for a three-dimensional reference coordinate system, receiving the medical image can include receiving at least two medical images comprising the first flexible elongate instrument disposed in the body lumen. The location information sensor can be a single element sensor

The system can further include a direction sensor configured to detect advancement and retraction of the relative movement of the first and second flexible elongate instruments.

The composite image further can include a representation of a treatment delivered to at least one of the plurality of vessel locations. The composite image can include a simulated representation of a location of the diagnostic or therapeutic device with respect to the medical image. The simulated representation can provide for a dimensional representation of the diagnostic or therapeutic device with respect to the lumen.

A method for locating a medical device in a body lumen includes establishing a reference coordinate system based on a plurality of imaging markers of a first flexible instrument disposed in a body lumen, the imaging markers being visible in a medical image comprising the first flexible elongate instrument. The method further includes receiving diagnostic scan or therapeutic delivery information at a plurality of locations of the body lumen from the first flexible elongate instrument or a second flexible elongate instrument configured for relative movement with respect to the first flexible elongate instrument (e.g., parallel, relative movement). At least one of the first and second flexible elongate instruments includes a location information sensor. The method further includes correlating the diagnostic scan or therapeutic delivery information with the imaging markers for the plurality of locations based on the reference coordinate system and location information as sensed by the location information sensor. A composite image comprising the correlated diagnostic scan or therapeutic delivery information and the imaging markers is displayed.

Optionally, the method can further include receiving the medical image comprising the first flexible elongate instrument disposed in a body lumen.

The location information sensor can be a sensor configured to detect encoding markers, and the method can further include detecting encoding markings of one of the first and second flexible elongate instruments.

The location information sensor can be a diagnostic sensor disposed on the second flexible elongate instrument, and the method can further include detecting a signal emitted by the first flexible elongate instrument. Correlating the diagnostic scan information with the imaging markers can include establishing a co-position location based on the detected signal.

The location information sensor can be a sensor that detects a push distance, a pullback distance, or a combination thereof of the diagnostic device, and one of the first and second flexible elongate instruments can include the diagnostic device. Correlating the diagnostic scan information with the imaging markers can include establishing a start location of a diagnostic sensor of the diagnostic device based on a relative position of the diagnostic sensor to at least one of the plurality of imaging markers.

The second flexible elongate instrument can be a diagnostic device comprising at least one imaging marker, and correlating the diagnostic scan information with the imaging markers can include establishing a start location of a diagnostic sensor of the diagnostic device based on a relative position of at least one imaging marker of the diagnostic device and at least one of the plurality of imaging markers of the first flexible elongate instrument.

The method can further include receiving directional information from a direction sensor configured to detect advancement and retraction of the relative movement of the first and second flexible elongate instruments.

A system for measuring relative displacement of at least two flexible elongate instruments within a body lumen includes a first flexible elongate instrument comprising a plurality of displacement encoding markers and a second flexible elongate instrument comprising an encoding sensor configured to obtain a signal from the displacement encoding markers. The encoding sensor is disposed at a distal portion of the second flexible elongate instrument and is configured for insertion into the body lumen. The first and second flexible elongate instruments are configured for relative movement (e.g., relative, parallel movement).

A processor in operative arrangement with the encoding sensor can be configured to determine relative displacement distances between the first and second flexible elongate instruments based on the obtained signal. The displacement encoding markers can be disposed at least partially circumferentially about a surface of the first flexible elongate instrument and comprise a reflective medium. The reflective medium can be or include a metal, metal alloy, magnet, ceramic, crosslinked hydrogel, fluoropolymer, or any combination thereof. The surface can be an inner circumferential surface of a catheter or a liner of the first flexible elongate instrument. Alternatively, or in addition, the surface can be an outer circumferential surface of a wire of the first flexible elongate instrument.

At least one of the first and second flexible elongate instruments can include a diagnostic device. The diagnostic device can be configured to obtain body lumen information. The processor can be further configured to correlate the obtained body lumen information and relative displacement distances. The body lumen information can include tissue density, temperature, pressure, flow rate, impedance, conductivity, or any combination thereof.

At least one of the first and second flexible elongate instruments can include a plurality of radiopaque markings. The processor can be further configured to receive at least one X-ray angiogram image of the body lumen comprising the plurality of radiopaque markings, correlate a first engagement position of the first and second flexible elongate instruments with at least one of the plurality of radiopaque markings of the X-ray angiogram image, and correlate a subsequent position of one of the first and second flexible elongate instruments to the at least one of the plurality of radiopaque markings of the X-ray angiogram image. A display can be configured to display a composite image comprising the radiopaque imaging markers and an indicator of the subsequent position or body lumen information obtained at the subsequent position.

The processor can be configured to continuously or periodically correlate subsequent positions of one of the first and second flexible elongate instruments to at least one of the plurality of radiopaque markings of the X-ray angiogram image. The display can be configured to continuously or periodically update the composite image with indicators of the subsequent positions or body lumen information obtained at the subsequent positions.

The system can further comprise a drive unit in operative arrangement with at least one of the first and second flexible elongate instruments. The drive unit can be configured to advance and/or retract the flexible instrument(s) within the body lumen. A processor can be configured to determine a relative displacement distance between the first and second flexible elongate instruments based on the obtained signal and generate a control command for the drive unit based on the determined relative displacement distance and a target location.

The system can include a processor configured to determine a relative displacement distance between the first and second flexible elongate instruments based on the obtained signal. The system can further include a display. The display can be configured to display a composite image that includes a representation of the body lumen and an indicator of a location of at least one of the first and second flexible elongate instruments within the body lumen.

An absolute position encoder system includes a member comprising a position encoder track comprising alternately spaced code lines of high and low reflectance, a light source configured to illuminate the encoder track, and an optical detector. The optical detector includes a single element light sensor configured to detect the encoder lines when the member is adjacent to the optical detector and moving relative to the optical detector, the single element light sensor detecting light reflected from a detection area of finite width. At least one code line of the position encoder track is of equal or greater width than the finite width of the detection area. At least one code line of the position encoder track is of narrower width than the finite width of the detection area. The optical detector generates an optical signal indicative of varying intensities. The system further includes a processor configured to translate the optical signal to code characters and measure an absolute position of the member based on the code characters.

The alternatively spaced code lines can provide for at least three light reflection levels. The optical detector can be in contact with the position encoder track. The optical detector can be disposed at a first endoluminal medical instrument, and the position encoder track can be disposed at a second endoluminal medical instrument. For example, the first endoluminal medical instrument can be a guidewire, and the second endoluminal medical instrument can be a catheter.

The optical detector can be detachably coupled to an endoluminal medical instrument and/or detachably coupled to a unit comprising the processor. The optical detector can include an optical fiber configured to transmit light from the light source to the encoder track and to transmit light reflected from the encoder track to a light intensity meter. Optionally, the alternately spaced code lines of high and low reflectance can be configured to provide directional information. At least one of the member and a component housing the optical detector further comprises a direction sensor.

An absolute position encoder system includes a member comprising a position encoder track comprising code lines engraved on a surface and an optical detector comprising an optical fiber communicatively coupled to an optical coherence tomography (OCT) instrument or an optical light reader. A tip of the optical fiber is disposed at a detection area and is configured to detect an engraved depth of each code line when the member is adjacent to the optical detector and moving relative to the optical detector. The optical detector generates an optical signal indicative of varying engraved depths. The system further includes a processor configured to translate the optical signal to code characters and measure an absolute position of the member based on the code characters.

The position encoder track can include code lines of at least three different depths. The surface of the position encoder track can be cylindrical, and the code lines can be circumferentially engraved on the surface. The optical detector can be in contact with the position encoder track. For example, the optical detector can be disposed at a first endoluminal medical instrument, and the position encoder track can be disposed at a second endoluminal medical instrument. The first endoluminal medical instrument can be a catheter and the second endoluminal medical instrument can be a guidewire.

The optical detector can be detachably coupled to an endoluminal medical instrument and/or detachably coupled to a unit comprising the processor. Optionally, the code lines can be configured to provide directional information. At least one of the member and a component housing the optical detector can further include a direction sensor.

A method of determining an absolute position, direction of motion, or speed of motion of a medical device inserted into a subject includes, with an absolute position encoder system: detecting an optical signal comprising at least two reflective intensities or at least two engraved depths as the member translates relative the optical detector, at least one of the optical detector and the member disposed at the medical device. The method further includes identifying an absolute position, direction of motion, or speed of motion of the medical device based on a time and duration of the at least two reflective intensities or at least two engraved depths.

A guidewire includes a plurality of radiopaque imaging markers, an embedded optical fiber; and a single element sensor disposed at a distal portion of the guidewire and operatively coupled to the optical fiber. The single element sensor is configured to detect location information encoding of a flexible elongate device.

The devices, systems, and methods provided are generally described within the context of X-ray applications, where the medical image can be an X-ray image or video (e.g., an X-ray angiogram, a computed tomography (CT) image) and imaging markers can be radiopaque imaging markers. The devices, systems, and methods provided can alternatively, or in addition, be used within the context of other imaging and sensing modalities. For example, a medical image can be a magnetic resonance (MR) image, including an MR-derived angiogram, and imaging markers can be MR-visible markers. A medical image can be a positron emission tomography (PET) image, or other radionucleotide-derived image, and imaging markers can be radiation-emitting markers. A medical image can be an ultrasound image, and imaging markers can be passive or active acoustic markers. A medical image can be obtained by an optical, thermal, and/or photoacoustic modality, and the imaging markers can be detectable or visible by the modality. A medical image can include a hybrid image generated from at least two imaging modalities. For example, the methods and systems can make use of or include multimodality sensor acquisitions (e.g., MR/PET), with a medical image being a multimodality image and imaging markers being multi-modality-visible markers.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic of an example system for locating a medical device in a body lumen.

FIG. 2A depicts simulated images obtained from an example IVUS scan and FFR scan without the benefit of a congruent location system.

FIG. 2B depicts simulated images generated from an example IVUS scan and FFR scan with the benefit of an example congruent location system.

FIG. 3 is a flow diagram depicting a standard diagnostic process and a process for congruent location measurement among multiple modalities.

FIG. 4 is a schematic of an example device in which a diagnostic sensor and imaging markers are disposed on a same flexible elongate instrument.

FIG. 5A is a schematic of an example device in which imaging markers are disposed on a guidewire for use with separate flexible elongate instrument that includes a diagnostic sensor.

FIG. 5B is a simulation of an X-ray angiogram image obtained with the device of FIG. 5A.

FIG. 6 is a schematic of an example device in which a flexible elongate instrument with imaging markers includes a location signal emitter (e.g., an ultrasound transducer).

FIG. 7 is a schematic of another example device in which a flexible elongate instrument with imaging markers includes a location signal emitter (e.g., an optical light emitter).

FIG. 8 is a simulation of an IVUS image obtained during co-location with the device of FIG. 6.

FIG. 9A is a schematic of an example flexible elongate instrument with radiopaque markers of differing size.

FIG. 9B is a schematic of another example flexible elongate instrument with radiopaque markers of differing size.

FIG. 10 is a flow diagram depicting an example coronary intervention procedure with use of the device of FIG. 4.

FIG. 11 is a flow diagram depicting an example coronary intervention procedure with use of the device of FIG. 5A.

FIG. 12A is a schematic of an example device in which a flexible elongate instrument includes a sensor for detection of displacement encoding markers.

FIG. 12B is a schematic of an example optical encoding liner for use with the device of FIG. 12A.

FIG. 13A is a schematic of an example system that includes a flexible elongate instrument having an optical encoding sensor and a liner having encoding markings.

FIG. 13B is a graph of an example signal and output produced with the system of FIG. 13A.

FIG. 13C is a schematic of another example of an optical liner encoder and an associated signal produced for displacement measurement.

FIG. 14 is a schematic of another example system in which displacement encoding markers of a flexible elongate instrument are detected for displacement measurement.

FIG. 15 is a schematic of an example system in which a flexible elongate instrument having an encoding sensor is used in conjunction with a therapeutic device delivering an angioplasty balloon.

FIG. 16 is a simulation of an example display including composite images generated with the benefit of displacement encodings that can be used to guide advancement of a catheter without live X-ray guidance.

FIG. 17 is a flow diagram depicting a standard treatment process with live X-ray guidance versus a treatment process guided with a system that includes flexible elongate instrument(s) with encodings and image markers.

FIG. 18 is a schematic of an example system used within a catheterization laboratory.

FIG. 19 is a simulation of a composite image resulting from co-location with an angiogram image with a guidewire model and a therapeutic/diagnostic device position within a lumen.

FIG. 20 is a block diagram of an example model generation process.

FIG. 21 is a diagram illustrating a 2D to 3D guidewire modeling construction.

FIG. 22 is a block diagram of an example data processing architecture.

FIG. 23 is a flowchart of a guided procedure workflow with a co-location system.

FIG. 24 is a flowchart of a workflow for imaging and lumen position correlation.

FIG. 25 is an example display of a co-location system with position co-location among multiple modalities.

FIG. 26 is a flowchart of a workflow for treatment and lumen position correlation.

FIG. 27 is a flowchart of a typical percutaneous intervention workflow.

FIG. 28 is a flowchart of a percutaneous intervention workflow with an example co-location system providing guidance.

FIG. 29 is a block diagram of a co-location system and communication overview.

FIG. 30 is a schematic of a prior art multi-track code for absolute position encoding and an example resulting signal in amplitude versus time.

FIG. 31 is a schematic of a prior art single-track code for absolute position encoding with an array-type sensor.

FIG. 32A is a schematic of an example detector including a single light-sensitive element for detecting single-track code.

FIG. 32B is a schematic of another example detector including a single light-sensitive element for detecting single-track code.

FIG. 33 is a diagram illustrating an example of determining absolute position with a single element sensor and an example resulting signal.

FIG. 34 is an example of a signal resulting from use of a device as shown in FIG. 32A or 32B and the encoding detection as shown in FIG. 33. The example signal includes detection of random speed movements and includes four direction changes.

FIG. 35A is a schematic of an example code track for detection by a single light-sensitive element.

FIG. 35B is a graph of example signals produced from the code track of FIG. 35A.

FIG. 35C is a schematic of another example code track for detection by a single light-sensitive element.

FIG. 35D is a graph of example signals produced from the code track of FIG. 35C.

FIG. 35E a schematic of yet another example code track for detection by a single light-sensitive element.

FIG. 35F is a graph of example signals produced from the code track of FIG. 35E.

FIG. 36 is a schematic of an example system including two flexible elongate instruments (as illustrated, a guidewire and a monorail catheter) including a detector having a single light-sensitive element for detecting absolute-position encoding.

FIG. 37 is a schematic of an example optical system for position encoding detection of endoluminal instruments.

FIG. 38 is an example of a seven-bit encoding providing for absolute position detection and detection of changes in direction.

FIG. 39 is a graph of an example signal produced from a device having an encoding as shown in FIG. 38.

### DETAILED DESCRIPTION

A description of example embodiments follows.

Devices, systems, and methods for locating a medical device in a body lumen are provided. Such devices, systems, and methods can advantageously provide for improved accuracy over existing positioning methods and reduced radiation exposure for clinicians and patients. The example devices, systems and methods described herein are generally described within the context of percutaneous coronary intervention (PCI) procedures; however, the provided devices and systems can be applied to or used within the context of other types of endoluminal procedures, such as gastrointestinal procedures.

Intravascular diagnostic and therapeutic-delivery methods are often performed with the use of X-ray angiography to aid in the visualization of a blood vessel section of interest. When performing an intravascular diagnostic scan, a sensor receives vessel-specific information (e.g., vessel size, tissue morphology, pressure, density, or temperature) while moving longitudinally within the vessel and records the vessel-specific information at each interrogated section.

While standard X-ray angiography provides for a two-dimensional projection of an interrogated blood vessel from outside of the vessel, intravascular diagnostic modalities interrogate a vessel from within the vessel lumen, and such modalities can generate many thousands of location-specific data points during a diagnostic scan along a lumen/vessel segment.

A shortcoming of intravascular assessment modalities, such as Intravascular Ultrasound (IVUS), Optical coherence tomography (OCT) and Fractional Flow Reserve (FFR), is that it is difficult to identify vessel locations on an X-ray angiography image and associate the locations to corresponding locations from the intravascular diagnostic scan, and vice versa. Furthermore, some types of blood vessel observations, such as calcium deposits and locations of significant pressure changes, obtained during an intravascular diagnostic scan are often difficult to locate on an X-ray angiography image. A clinician may try to use features (e.g., a vessel branch, or severe vessel narrowing) that are detectable in both the X-ray angiography images and in the intravascular longitudinal diagnostic scan to help mentally identify the corresponding locations. However, there are no universal features present in all patients, making the process subject to clinician skill and experience.

Some X-ray equipment manufacturers provide for continuous monitoring during an angiography procedure, while device movement within the vessel during a vessel diagnostic scan is recorded. Post-processing calculations can be employed to correlate locations from intravascular scans to vessel locations on the obtained X-ray angiography images. However, such methods expose the clinician and patient to high X-ray radiation levels and do not provide a clinician with real-time correlation. Furthermore, generating a three-dimensional vessel model in this manner can be cumbersome, disruptive to clinician workflow, and inaccurate.

Interventional procedures performed under X-ray angiography guidance involve similar shortcomings. Once diagnostic imaging information is obtained (e.g., cross-sectional views, longitudinal views, and physiological indices), the imaging probe is withdrawn, and a therapeutic device (e.g., a catheter carrying a balloon or stent) is then deployed under X-ray fluoroscopy guidance. X-ray angiography is often required to locate a position of the guidewire and a position of the therapeutic and/or diagnostic device within the body vasculature because there is some amount of travel between the entry point and the target location, and linear distance tracking during insertion or pullback of a device is often inaccurate.

There is a need for facile methods of correlating vessel locations identified from intravascular diagnostic scans to vessel locations on X-ray angiography images. There is also a need for improved methods of measuring medical device displacement in a body lumen with reduced discrepancy between measured displacements and actual device displacements in the body. There is a further need that such methods significantly reduce radiation exposure to patients and clinicians over existing continuous X-ray angiography procedures.

An example system for locating a medical device in a body lumen includes a first flexible elongate instrument 110 and, optionally, a second flexible elongate instrument 112 configured for parallel, relative movement with respect to the first flexible elongate instrument. The first flexible elongate instrument includes a plurality of imaging markers 130a-130d, which can be, for example, radiopaque imaging markers. A location information sensor 120, 126 can be disposed at the first flexible elongate instrument 110. For example, a location information sensor 120 can be disposed on or in the first flexible elongate instrument at a distal portion of the instrument, and/or a location information sensor 126 can be disposed at a proximal portion of the instrument (e.g., a push and/or pullback sensor, which can optionally be, or be a component of, a drive unit configured to advance and/or retract the instrument), which remains located outside a patient. Alternatively, or in addition, a location information sensor 122 can be disposed at the second flexible elongate instrument. As illustrated, the location information sensor 122 of the second flexible elongate instrument is disposed at a distal portion of the instrument; however, it can alternatively be disposed at a proximal portion (e.g., a push and/or pullback sensor, similar to sensor 126). The first flexible elongate instrument 110 can be, for example, a guidewire, a wire including a diagnostic sensor (e.g., an FFR wire), a wire including a therapeutic device (e.g. an atherectomy wire). The second elongate instrument 112 can be, for example, a catheter (e.g., an IVUS or OCT catheter, a balloon delivery catheter, a catheter of a biopsy device or aspiration device, an endoscopic catheter, etc.). Examples of various arrangements of location information sensor(s) 120, 122, 126, of FFR, IVUS, and OCT diagnostic implementations of the system 100, and of therapeutic delivery implementations of the system 100 are further described in Sections 1-4 herein.

The system further includes a processor 105 and a display 107. The processor 105 can optionally receive at least one medical image that includes the first flexible elongate instrument 110 disposed in a body lumen. In addition, or alternatively, the medical image can be received by a separate system processor and independently displayed. The processor is configured to establish a reference coordinate system based on the plurality of imaging markers 130a-d, which are visible in the medical image, and receive diagnostic scan or therapeutic delivery information at a plurality of locations of the body lumen from the first or second flexible elongate instrument. The processor is further configured to correlate the diagnostic scan or therapeutic delivery information with the imaging markers for the plurality of locations based on the reference coordinate system and location information as sensed by the location information sensor. The medical image can be, for example an X-ray image, such as an X-ray angiography image.

As used herein, the term “medical image” is intended to include any image produced by a medical imaging system for the viewing of internal anatomy of a patient. Medical images can be obtained from, for example, magnetic resonance (MR) imaging, nuclear magnetic resonance (NMR) imaging, computed tomography (CT), X-ray, and positron emission tomography (PET), among other imaging modalities. A medical image can include one or more static images. For example, a medical image can be an ultrasound video.

As used herein, the term “X-ray image” is intended to include any image produced by X-rays being passed through a body, including, for example, an X-ray angiography image, an X-ray fluoroscopy image, and a computed tomography (CT) image. An “X-ray image” can include one or more static images. For example, an “X-ray image” can be an angiography video comprising a plurality of images.

While the system 100 is generally described with regard to radiopaque markings and X-ray images, the system 100 can alternatively provide for use with other imaging modalities, including, for example, magnetic resonance (MR) imaging, nuclear magnetic resonance (NMR) imaging, and positron emission tomography (PET). For such modalities, the markings 130a-d can be modality-specific markers. For example, the markings 130a-d can comprise an MR-sensitive or NMR-sensitive (e.g., comprises atoms with a free nuclear spin), electromagnetic sensitive, electromechanical sensitive, optically sensitive, and/or mechanically sensitive material that is detectable or distinguishable in the image. Instead of an X-ray image, an MR, NMR, or PET image, among other modalities, can be obtained by the processor 105 for correlation with the diagnostic scan or therapeutic delivery information.

As used herein, the term “reference coordinate system” includes one-dimensional, two-dimensional, and three-dimensional spatial reference systems in which at least one location (typically an initial location) of the first flexible elongate instrument is registered with respect to the imaging markers, which are visible on a medical image, and upon which subsequent positions of the first or second flexible elongate instrument are determined. Examples of establishing 1D, 2D and 3D reference coordinate systems to provide for location determination during an endoluminal diagnostic scan or therapeutic intervention are further described in Sections 1-3 herein. For example, establishing a 1D reference coordinate system can include registering an initial location of a flexible elongate instrument in the vessel with respect to the imaging markers. For a further example, establishing a 2D or 3D reference coordinate system can include generating a model of the imaging markers and, optionally, the vessel lumen, based on a representation of the imaging markers in one or more medical images (e.g., one or more X-ray angiogram images).

As used herein, the term “diagnostic scan or therapeutic delivery information” includes any information obtained during a diagnostic scan or during delivery of a therapeutic intervention, including, for example, information pertaining to a location of a diagnostic sensor or therapeutic device, a reading by a diagnostic sensor, and an image obtained by a diagnostic device.

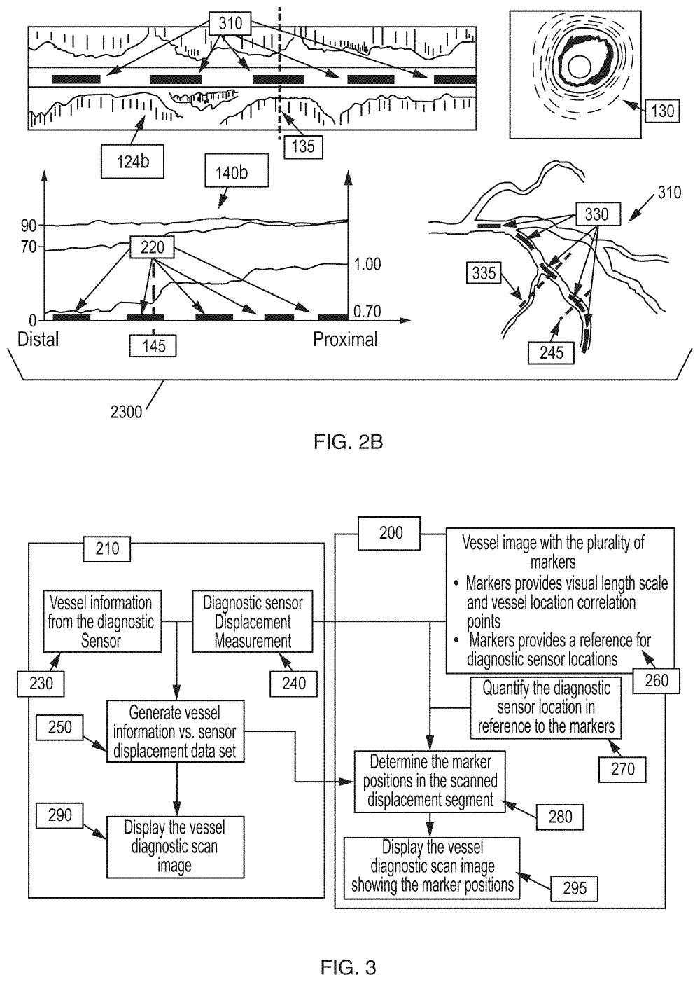

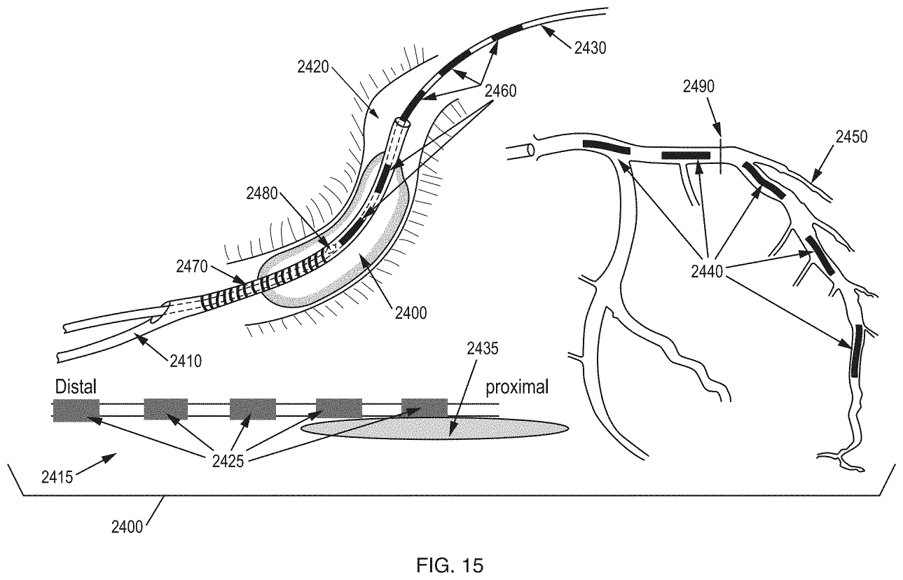

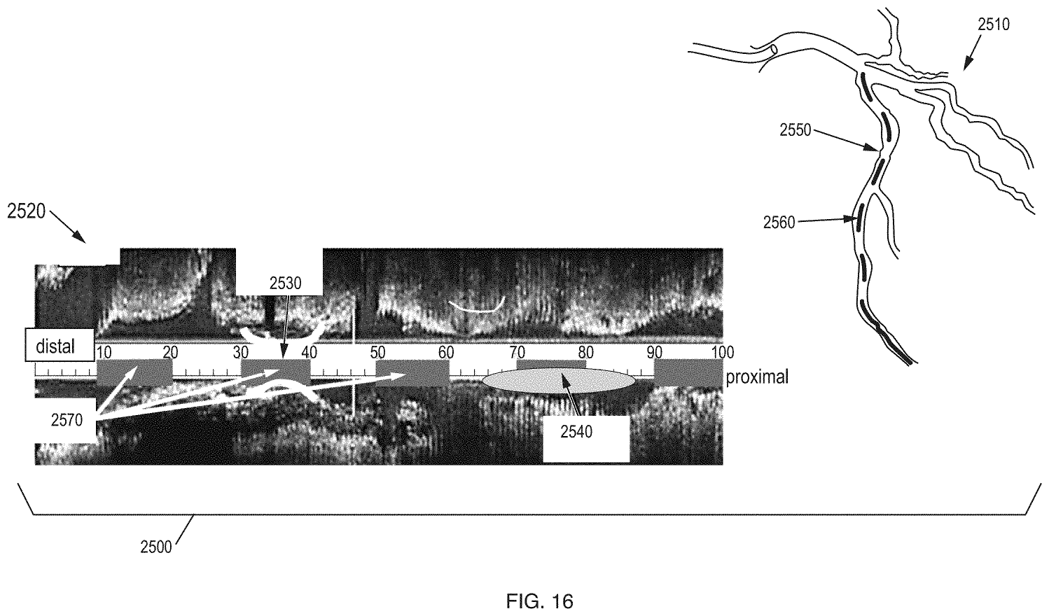

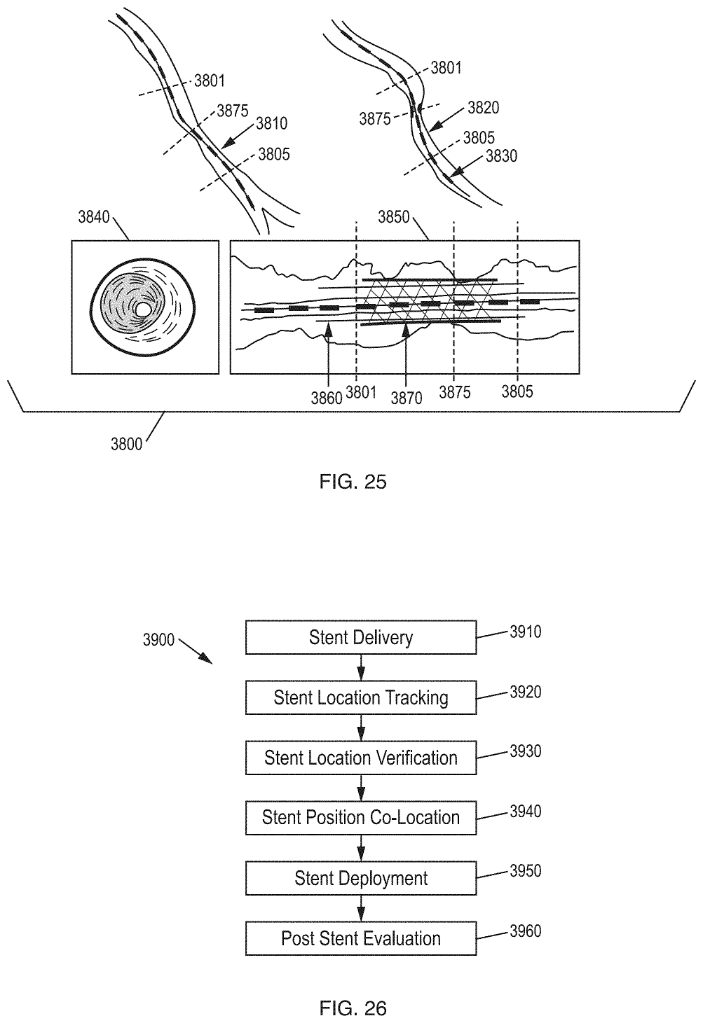

The display 107 is configured to display a composite image comprising the correlated diagnostic scan or therapeutic delivery information and the imaging markers. The composite image can be, for example, an image or graph obtained from the diagnostic scan, such an OCT image or an FFR graph, on which a representation of the imaging markers is superimposed (see, e.g., display 124b, 140b of FIG. 2B, display 2415 of FIG. 15, FIG. 16). The composite image can be, in another example, the X-ray image on which a representation of a location of the diagnostic or therapeutic device is superimposed (see, e.g., display 310 of FIG. 2B, display 2450 of FIG. 15, FIG. 16, FIG. 19). The composite image can, in a further example, include an image in which information from multiple modalities or of multiple device positions are indicated (see, e.g., display 20 of FIG. 2B, display 2400 of FIG. 15, FIG. 16, FIG. 19, FIG. 25). The composite image can include a representation of the body lumen in which the first and, optionally, second flexible elongate device is disposed and an indicator of a location of the device(s) (e.g., FIG. 19, FIG. 25).

The methods and systems described herein can advantageously provide for significant reductions in X-ray exposure as compared with typical PCI procedures. Conventional PCI methods not only rely on constant real-time or about real-time X-ray angiography and fluoroscopy feeds for device displacement measurement and location tracking, but are also not able to offer real-time, precise location correlation across a full range of device tool sets applied throughout a PCI procedure. Conventional methods thus involve high levels of radiation exposure to the patient and/or clinician. Furthermore, a lack of real-time or about real-time positional correlation between the angiogram, diagnostic modalities, therapeutic devices, and associated diagnostic measurements often results in additional X-ray imaging, contrast, and time, thereby further increasing radiation exposure and compromising strategy decisions and treatment outcomes throughout the PCI procedure.

Current PCI procedures heavily rely on real-time or about real-time fluoroscopy. Because the images are taken in real-time throughout the procedure, substantially greater amounts of X-ray radiation are required as compared to a single radiograph (e.g., an image for bone fractures). There are known exposure thresholds for tissue injury that are relevant to patients such as skin erythema (˜2 Gy) and permanent skin injury (˜5 Gy). For operators, the eye lens is susceptible, and a risk of cataracts increases with acute exposure as low as 0.1 Gy and chronic exposure of 5 Gy. Stochastic effects, including cancer, involve a long latency period, and a lifetime attributable risk is also presented, though difficult to quantify. Because of the radio-sensitivity of tissues, child patients and patients with preexisting health conditions are presented with a higher radiation safety risk during PCI procedures. Angiography uses radiopaque contrast agents to image the vasculature. In addition to the X-ray exposure, patients may suffer side effects from the radiopaque contrast agents, including pain, adverse drug interactions, and renal failure. For physicians and staff, there are also risks of X-ray exposure as well as orthopedic injuries (e.g., lower back strain) due to the extra weight of the lead-lined aprons and other protective equipment.

The methods and systems described herein allow for a reduced X-ray exposure to the patient and/or the operator when performing PCI procedures. Excessive X-ray exposure is toxic to the human body, with co-morbidities such as cancer, hair loss, and cataracts. While a conventional X-ray dose baseline varies depending upon the nature of a procedure, human factors, X-ray equipment, staff dose registry accuracy, etc., on average, a baseline X-ray exposure ranges from about 3 to 5 Gy (Grays) for a procedure that takes about 20 minutes to about 15 Gy for PCI procedures. The methods of the present disclosure can provide for PCI procedures in which a significant reduction in overall X-ray dosage can be achieved as a result of reducing the X-ray “on” time during the PCI procedure. The X-ray “on” time of the methods described herein can be reduced by up to 10, 20, 30, 40, 50, 60, 70, 80, 90, or 95% relative to a conventional PCI procedure. An X-ray dosage received by a patient during the PCI methods described herein can range from less than 500 mGy for a PCI procedure lasting about 20 minutes (reduced from about 3 to 5 Gy) to about 2 Gy for a complex procedure (reduced from about 15 Gy). An X-ray dosage received by the patient during the PCI procedure can be less than about 500 mGy, less than about 400 mGy, less than about 300 mGy, less than about 200 mGy, or less than about 100 mGy.

### 1. BODY LUMEN LONGITUDINAL LOCATION METHODS AND SYSTEMS

Endoluminal procedures typically require the use of X-ray, often with the aid of contrast agent injection, to allow a user (e.g., a physician) to visualize a vessel such that a guidewire and additional intravascular devices can be located and steered to a correct vessel branch. X-ray with the use of a contrast agent can also be useful as a preliminary diagnostic scan of a vessel/tissue condition. Often, additional diagnostic procedures involving other modalities (e.g., intravascular IVUS, OCT, and FFR) are performed for more critical evaluation of disease conditions. In a typical operating room in which catheterization is performed, an X-ray angiography image and other additional imaging modalities are displayed either on different screens or on a large panel screen within different partitions. Vessel locations observed from an intravascular diagnostic modality such as IVUS, FFR or OCT screen for example, are not correlated to X-ray vessel images, and vice versa.

Guidewires with a plurality of radiopaque markers with known spacing have been used to provide for length estimations of vascular or internal body lumen features on X-ray images. However, such markers are not correlated to other diagnostic scan information, such as IVUS, OCT, and FFR.

Typically, intravascular diagnostic systems combine blood vessel diagnostic information obtained from an ultrasound transducer, such as in an IVUS system, from optical transducers, such as in an OCT system, or from pressure transducers, such as in an FFR system, with a displacement tracking unit, such as a motor drive unit, to generate intravascular displacement scan images. The diagnostic sensor (e.g., ultrasound transducer, optical transducer, or pressure sensor) is placed inside a vessel under real-time X-ray angiography guidance. During a vessel diagnostic scan, thousands of vessel diagnostic data points are generated, each corresponding to a measured displacement point. However, the vessel location of each displacement point is not quantified because there is no vessel location reference system in a body lumen to quantify a sensor location or the location of a data point generated by the sensor. Even if the sensor is detectable on X-ray and the starting point of the sensor during an intravascular scan is detected by an X-ray angiography image, the lack of vessel length scale and the two-dimensional projection nature of a three-dimensional vessel in the X-ray angiography image makes it difficult to correlate vessel locations between the two-dimensional X-ray vessel image and the vessel diagnostic scan image, which is displayed with respect to a measured linear distance.

A flexible elongate instrument (e.g., flexible elongate instrument 110) having a plurality of radiopaque markers strategically located and visible on an X-ray angiographic image of a vessel can advantageously provide for fixed points along the body lumen from which a linear location reference system can be defined. The linear location reference system can enable location correlation among the X-ray angiography image, diagnostic scan images or graphs, and/or therapeutic delivery devices. The flexible elongate instrument can remain at a same position in the body lumen such that subsequent positioning of additional flexible elongate instruments within the body can be correlated with or without real-time X-ray angiography.

FIG. 2A illustrates an example display 10 of images obtained from a typical endoluminal procedure that includes IVUS and FFR scans and without the benefit of a congruent location system. The display includes an X-ray angiography image 110 of coronary vessels. Contrast agent, commonly an iodine solution, is injected into vessel sections of interest such that the vessels are detectable on the X-ray angiography images. Catheter devices disposed inside of a vessel are typically not clearly detectable in the X-ray angiography image without radiopaque markings.

The display 10 further includes a longitudinal IVUS pullback scan view 124a, from which dimensional and morphological vessel features obtained by the ultrasound sensor are displayed with respect to the pullback scan distance, as detected by a pullback sensor disposed externally of the body. The display further includes a cross-sectional IVUS view 130 of the vessel, illustrating lumen size and morphology at the dashed line 135 in view 122. Current IVUS and OCT systems are equipped such that a lumen cross section view can be displayed at any displacement location chosen by a user with respect to the longitudinal view. IVUS sensors rotate during pullback, generating 360-degree views of vessel morphology along a scanned length of the vessel.

The display 10 further includes a longitudinal FFR pullback scan view 140a of the vessel from which the fractional reserve ratio (e.g., a ratio of vessel pressure at a distal location vs. aortic pressure), is displayed against a length of the scan distance.

The vessel lumen information obtained from IVUS and FRR during a displacement scan provides clinicians with more relevant diagnostic information of a vessel segment of interest than from X-ray angiography alone.

The data sets generated by IVUS, OCT, FFR, and other intravascular scan modalities typically register vessel information with respect to a longitudinal pullback displacement. This type of data set is based on linear distance and lacks three-dimensional vessel curvature information. The fact that an X-ray vessel image is a two-dimensional projection of a three-dimensional vessel makes distance judgments among, for example, views 110, 124a, 130, 140a even more difficult.

While viewing the longitudinal pullback scan views 124a and 140a alone, it is difficult to correlate a location from these scan images to a vessel location on the X-ray angiography image 110. Often, even when a scan starting point is identified, it is still difficult to point to an angiographic vessel location with a defined distance from the starting point due to the 2D projection effect of the X-ray angiography image. The IVUS vessel scan location marked by dashed line 135, for example, does not include any clear references that can be used to correlate the location to an angiographic vessel location in view 110.

Similarly, an FFR pullback scan location indicated by dashed line 145, where a change in FFR ratio is observed, is also difficult to correlate to a location shown on the X-ray angiographic vessel image 110.

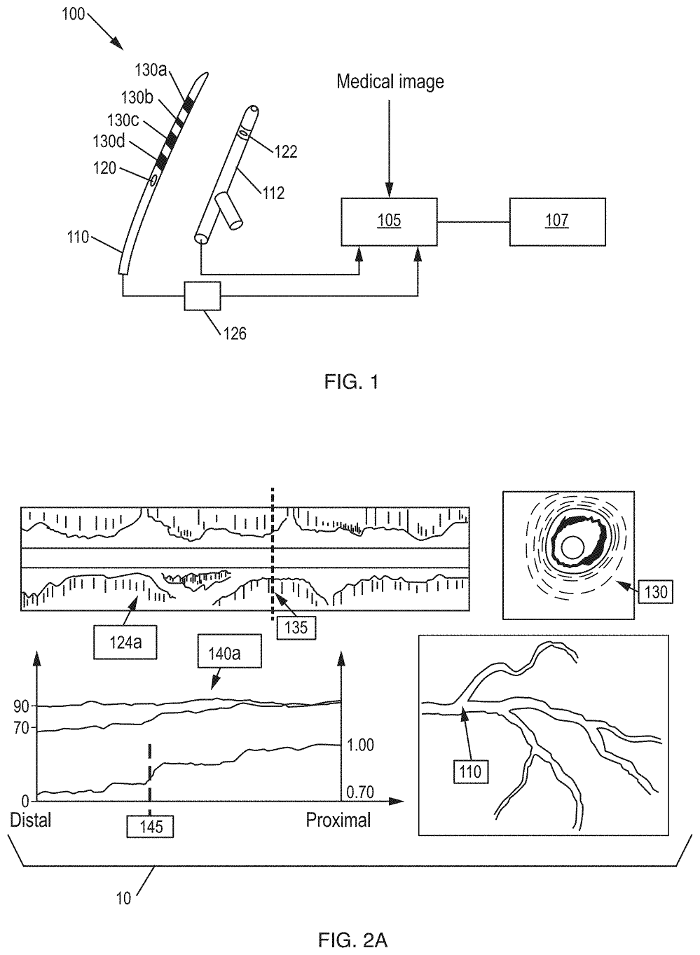

FIG. 2B illustrates an example display 20 of images obtained from an endoluminal procedure that includes IVUS and FFR scans, with the benefit of a congruent location system. A flexible elongate instrument, such as the flexible elongate instrument 110 (FIG. 1) is disposed within a vessel, and an X-ray angiography image 310 is obtained that includes a visualization of the radiopaque markers 330 of the instrument. The locations of the markers in relationship to the vessel (as detected by the X-ray angiographic image) are projected onto the longitudinal IVUS pullback scan view 124b as markers 310 and onto the longitudinal FFR pullback scan view 140b as markers 220.

The IVUS vessel scan location indicated by the dashed line 135 can now be correlated to an X-ray angiographic vessel location indicated by the dashed line 335. It can be easily inferred that the IVUS vessel cross sectional view 130 is located at the position of dashed line 335 in the X-ray angiographic view.

Similarly, the FFR pullback scan location indicated by dashed line 145 can be easily correlated to a location indicated by the dashed line 245 on the X-ray angiographic vessel image 310.

FIG. 3 is a flow diagram depicting a method that includes a standard diagnostic process 210 and a process for congruent location measurement 200 among multiple modalities to obtain correlation information as shown in FIG. 2B. During a vessel diagnostic scan, vessel information 230 from a diagnostic sensor and sensor displacement information 240 are combined to generate a dataset 250 that includes vessel information vs. sensor displacement. The data set is then displayed 290 (e.g., views 124a, 130, 140a of FIG. 2A). In the displayed image, the vessel diagnostic information is displayed relative to the sensor displacement. The vessel diagnostic information at any sensor displacement point is not correlated to any vessel location seen on an X-ray angiography image of the vessel.

With the additional functions depicted in method 220, accurate correlation of sensor displacement points to vessel locations on an X-ray vessel image can be provided. To provide for accurate location correlation to an X-ray angiography image, the image can include a vessel length scale and vessel location correlation points, both of which can be provided by the radiopaque markings of a flexible elongate instrument, such as instrument 110 (FIG. 1). In particular, an X-ray angiography image is obtained 260 with a flexible elongate instrument in place in the vessel of interest. The plurality of markers of the instrument, which are detectable within the vessel, provide for both a vessel length scale and visual reference point(s) for vessel location correlation. The markers, as visualized on the X-ray angiography image, are particularly useful as vessel location references because an X-ray angiography image is a 2D projection of a vessel segment in a 3D space and linear length scales in the diagnostic image are not directly translatable to positions as seen on the 2D projection. The plurality of markers can also provide for quantifying a location of a diagnostic sensor.

Once a diagnostic sensor's location relative to the plurality of markers is quantified 270, the positions of the plurality of markers in the scanned displacement segment can be measured 280 and projected onto a composite image 295 for display (e.g., views 124b, 140b in FIG. 2B).

The provided method 220 does not specify a sequence among items 240, 260, and 270. Depending on the devices and instruments used in a particular application, there are many ways to quantify a sensor location in reference to the plurality imaging markers. Generally, when a sensor location is quantified in reference to at least one of the plurality of markers, the sensor position in a scanned length can be measured for various other locations. Examples of various methods of quantifying a sensor location depending upon an arrangement of the flexible elongate instrument(s) follow.

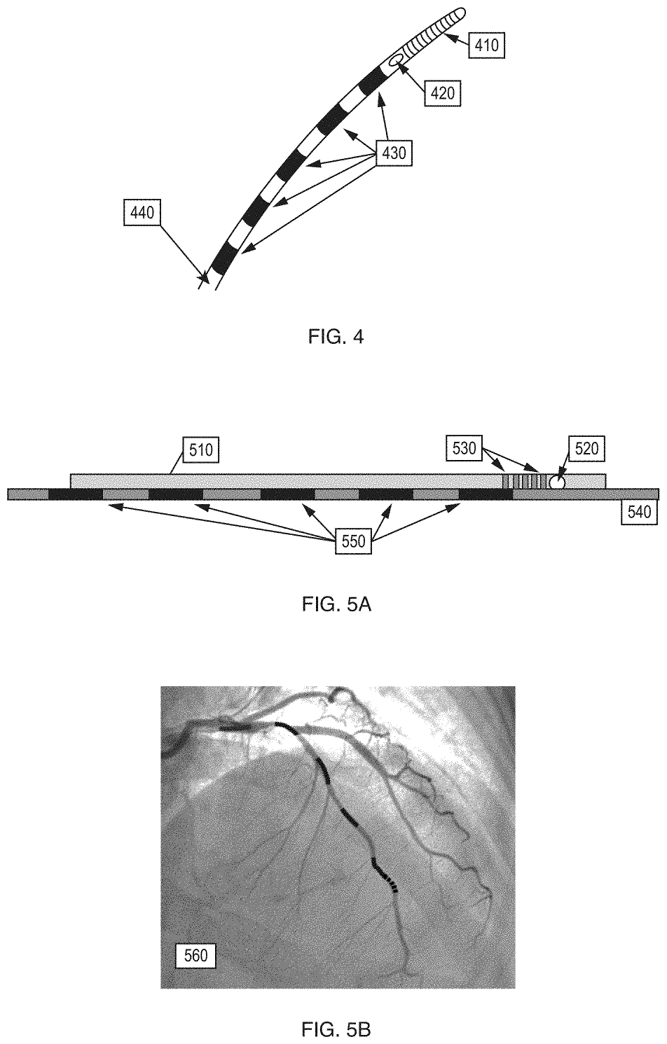

FIG. 4 depicts an example flexible elongate instrument 440 that includes both a diagnostic sensor 420 and radiopaque markers 430. The device 440 can be, for example, an FFR wire. When an X-ray angiography image is obtained, a position of the diagnostic sensor 420 relative to the markers 430 as detected by the X-ray angiography image is known and can thereby be initially quantified. For a device such as FFR wire 440, a location information sensor can be a sensor that detects a pullback distance of the wire (e.g., sensor 126, FIG. 1).

Clinicians often perform FFR pullback scans to better assess pressure changes within a section of a vessel that may have defused or that may include more than one lesion. Pullback scans can be performed by a motor unit positioned outside of the body, which records the pullback distance (e.g., pullback sensor 126, FIG. 1) and/or a push distance. A pressure sensor 420 is disposed in or on the FFR wire, just proximal to a flexible distal tip 410 of the wire. As illustrated, the radiopaque markers 430 are located at known distances proximal to the pressure sensor.

An X-ray angiography image of the vessel and markers can be obtained at any point along the pullback scan, provided the scan pullback distance where the X-ray angiography image is obtained is also recorded. Because the distances of the radiopaque markers to the sensor are already known and remain fixed, the sensor location relative to the markers when an X-ray angiography image is taken is therefore also known, and the marker locations can be calculated relative to the scan length and be projected onto the FFR diagnostic scan display.

In an example workflow with the device 440, an X-ray angiography image can be taken before an FFR diagnostic scan starts, and the known FFR sensor location relative to the markers can coincide with the scan starting point, or zero displacement point. The positions of the makers relative to the scanned length can be measured and displayed. To allow all markers on the instrument to be visualized on a pullback scan display and to provide for the widest range of location references, the location at which an X-ray angiography image is obtained can be substantially that at which the physical location of the sensor is most distal, generally at the start of the diagnostic scan.

FIGS. 5A and 5B depict an example system in which a flexible elongate instrument 540 includes radiopaque markers 550 and is used with a second flexible elongate instrument 510 having a diagnostic sensor 520. As illustrated in this example, the first flexible elongate instrument 540 is a guidewire. Each marker length and spacing between the markers 550 are known (e.g., 10 mm). The second flexible elongate instrument 510 is a diagnostic device with a diagnostic sensor 520 (e.g., an FFR wire).

Unlike the example shown in FIG. 4, a position of the diagnostic sensor 520 relative to the plurality of markers 550 cannot be measured based on the design of the instruments. Proximal to the diagnostic sensor 520, several radiopaque markers 530 are affixed to the shaft near the sensor (e.g., sensor disposed 1 mm from the distal most marker). In this example, the diagnostic sensor 520 is not detectable by X-ray, which is the case for most types of diagnostic sensors. The markers 530 are spaced relative to each other and to the sensor such that the distance of each marker to the sensor can be easily measured (e.g., marker lengths and spacing of 1 mm).

The guidewire-based markers 550 are configured to be easily distinguishable with that of the diagnostic-instrument-based markers 530. An X-ray angiography image 560 of the vessel is shown in FIG. 5B in which both the guidewire-based markers and the diagnostic-instrument-based markers are detectable. A position of the diagnostic sensor relative to the guidewire markers in this example can be measured (e.g. just over 8 mm distal to the distal most marker on the guidewire using the hypothetical parameters provided in this example).

In this example, the markers used for vessel reference are affixed to the guidewire, which does not need to move during a diagnostic intravascular scan. While a real-time or about real-time X-ray angiography image can be obtained during the diagnostic scan, vessel location correlation can instead be performed with a recorded X-ray angiography image.

Guidewire-based markers can also be useful when, subsequent to the initial diagnostic procedure, an interventional device or a vessel treatment device is inserted. As the markers remain in the vessel during both the diagnostic and interventional procedures, the markers can provide for improved correlation to diagnostic data during the interventional procedure and, optionally, can also be used to guide the interventional device to a desired vessel location under either real-time X-ray guidance or guidance with a pre-obtained X-ray.

With the example devices of FIG. 5A, a location information sensor can be a sensor that detects a pullback distance of the diagnostic device 510 (e.g., sensor 126, FIG. 1). As described above, correlating the diagnostic scan information with the X-ray angiogram image can be based on establishing a start location of the diagnostic sensor 520 based on a relative position of an imaging marker of the diagnostic device 530 and the radiopaque imaging markers 550 of the guidewire.

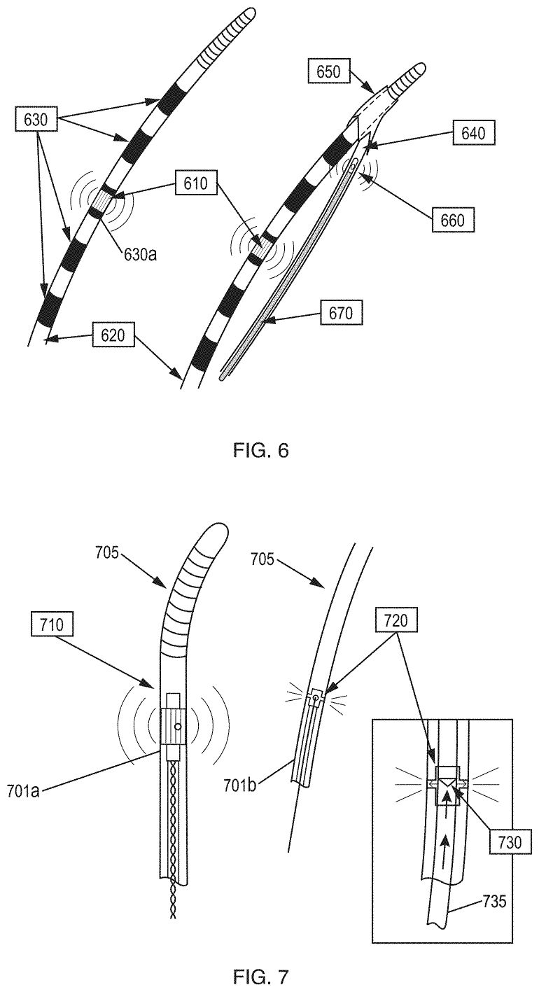

FIG. 6 depicts an example system in which a flexible elongate instrument 620 or 640 includes a location information sensor 610 or 660 disposed at a distal portion of the device. In the example to be described, the plurality of markers 630 and the diagnostic sensor 660 are located on different flexible elongate instruments, and a location of the diagnostic sensor relative to the markers as detected by the X-ray angiography image is not known.

In this example, a first flexible elongate instrument is a guidewire 620 with a plurality of markers 630 and including a signal emitter or transducer 610 of a modality that is detectable by a diagnostic sensor 660 located at a second flexible elongate instrument 640. As illustrated in this example, the second elongate instrument is a diagnostic catheter 640. The diagnostic catheter can be, for example, an IVUS catheter, an OCT catheter, or an FFR catheter. The signal emitter 610 can provide for co-location information in conjunction with the diagnostic device 640.

For an IVUS catheter, the guidewire signal transducer 610 can be an ultrasound transducer or a signal reflector. For an OCT catheter, the guidewire signal transducer 610 can be an optical-fiber-based emitter/receiver.

As illustrated in FIG. 6, the signal transducer 610 is disposed on the guidewire such that it coincides with a middle radiopaque marker 630a. However, the transducer 610 can be located at any location along a distal portion of the guidewire 620.

The radiopaque markers 630 can each be of a known length. For example, if each marker length and the gaps in between two markers are 10 mm, in this drawing, which shows 5 markers, a total indicated distance that can be viewed and precisely measured from X-ray angiography images is 90 mm.

The diagnostic catheter 640 can be, for example, a rotational IVUS catheter or an OCT catheter that has been inserted over the guidewire and permitted to move along the guidewire when advancing or retracting within a vessel. An over-the-wire sliding rail portion 650 of the catheter, often referred to as a catheter guidewire lumen, is situated at a distal tip of the diagnostic catheter 640. A guidewire lumen allows a catheter to be loaded onto a guidewire and follow the guidewire on insertion into a vessel. As illustrated, the diagnostic sensor 660 is mounted at a distal end of a rotational core 670 of the diagnostic device. During a diagnostic scan, the rotational core 670 rotates while the diagnostic device is pulled back by a motor drive unit, which also measures the device's displacement, generating a 360-degree diagnostic view of the vessel along the pullback length.

For IVUS, the diagnostic sensor 660 can be an ultrasound transducer (e.g., operating in 5-60 MHz range). For OCT, the diagnostic sensor 660 can be an optical sensor, for example, a small optical mirror that reflects beams of light 90 degrees from an optical fiber such that the light is projected perpendicular from the catheter.

During a diagnostic pullback scan, the rotating core 670 and the transducer 660 move proximally, generating a cross sectional image of the vessel with every rotation, which is registered with the pullback distance.

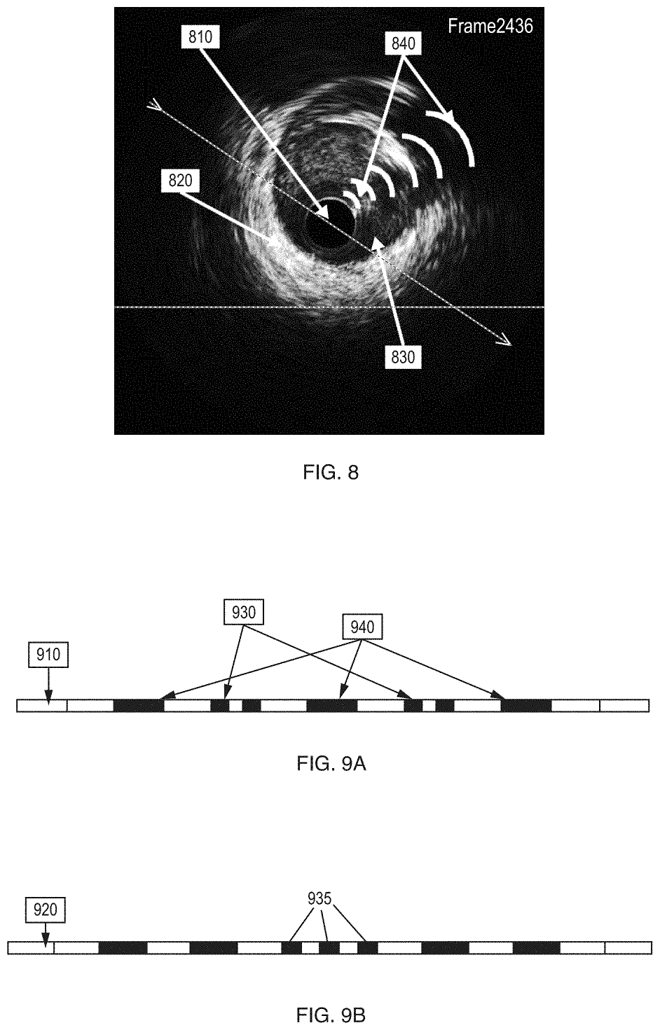

When the diagnostic sensor 660 passes by the guidewire signal transducer 610, the emitted signal from the guidewire transducer can be detected by the diagnostic sensor, or vice versa (see FIG. 8), and the pullback distance where the signal is detected can be recorded. Because the location of the guidewire transducer 610 relative to the plurality of markers is known, when the diagnostic sensor 660 detects the signal emitted by the transducer 610, the diagnostic sensor 660 location relative to the plurality of markers can be quantified. A determination of when the guidewire transducer and the diagnostic sensor are next to each other can be measured based on signal timing and/or signal strength. Once the diagnostic scan displacement point at which the diagnostic sensor 660 is next to the guidewire transducer 610 is calculated, a position of the plurality of radiopaque markers 630 relative to the diagnostic sensor can be established and projected onto the vessel diagnostic scan images.

A strength of the guidewire transducer emission can be adjusted, for example, to make it weak enough such that only the closest few frames register the signal, thereby providing for improved location accuracy. However, such an approach can provide for increased difficulty in detecting these few frames after the pullback scan is completed. Alternatively, the transducer emission can be adjusted to be stronger such that the signal can be more easily detected across a larger number of frames. However, such an approach can result in reduced location registration precision. To aid visualization, or to distinguish the guidewire transducer emission from actual reflected signals of the tissue anatomy, defined signal patterns can be emitted.

The guidewire-based transducer can be configured to act purely as a receiver and use timing of emission and reception for accurate location registration where the guidewire-based transducer and the diagnostic sensors are connected to a same system. This can advantageously avoid the generation of image artifacts in the images obtained by the diagnostic sensor. Signals with a smallest time differential can provide for detection of the position at which the guidewire transducer and diagnostic sensor are closest.

As illustrated in FIG. 6, the diagnostic sensor 660 can serve as a location information sensor. Correlating the diagnostic scan information with the X-ray angiogram image can include establishing a co-position location based on a signal-emitter 610 emitting a signal detectable by the diagnostic sensor 660. The signal-emitter can be, for example, an ultrasound transducer, an optical light emitter, or a characteristic signal reflector that can reflect a signal emitted by the sensor 660 for detection by the sensor 660.

FIG. 7 depicts additional examples of flexible elongate instruments that each include a signal emitter or receiver that is configured to emit a signal for detection by a diagnostic sensor or detect a signal emitted from the diagnostic sensor.

Flexible elongate instrument 701a is a guidewire (radiopaque markings not shown in FIG. 7) that includes an ultrasound transducer 710 disposed near a distal end 705 of the wire and configured for use with IVUS imaging catheters. Flexible elongate instrument 701b is a guidewire that includes an optical light emitter/receiver 720 disposed near a distal end 705 of the wire and configured for use with OCT image catheters.

Ultrasound transducers are typically mostly made of piezoelectric materials which, by nature, can function as both a signal emitter and signal receiver. IVUS catheters, depending upon an intended location of use in the body (e.g., coronary vessel, peripheral vessels, intracardiac applications, etc.) and vessel size, include transducers that operate at different frequencies. For example, an IVUS catheter can include a transducer operating in range of about 9 MHz for large body lumens to about 60 MHz for small body lumens. Guidewires of different diameters are also available for accessing vessel/lumens of different sizes. Transducers with different center frequencies can be used to suit different imaging catheter frequencies and guidewire diameters. For example, a 50 MHz ultrasound transducer made of PZT material can be approximately 30-50 microns in thickness. Such a transducer can be disposed on or in, for example, a guidewire having a diameter of about 300-400 microns without affecting the strength and physical properties of the guidewire.

An ultrasound transducer configured as such can emit/receive signals 360 degrees perpendicular to a length of the guidewire and can be designed such that the signal propagates in a narrow plane.

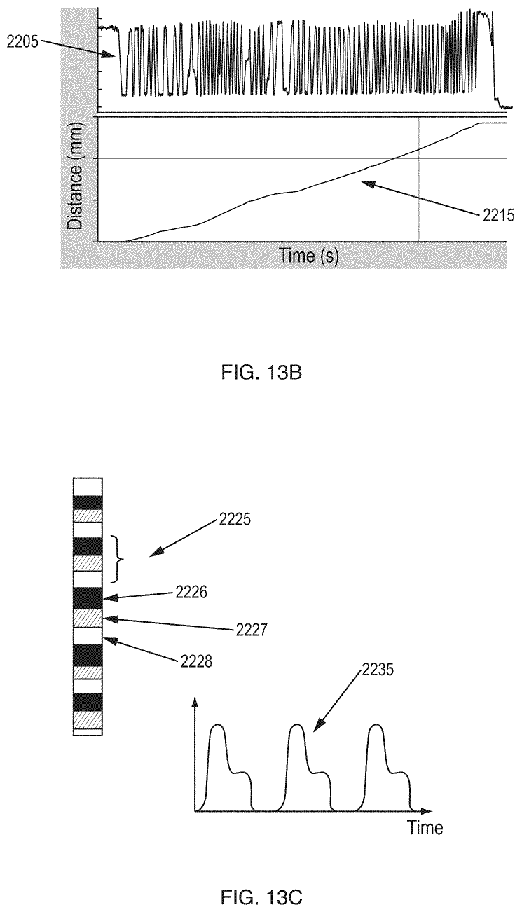

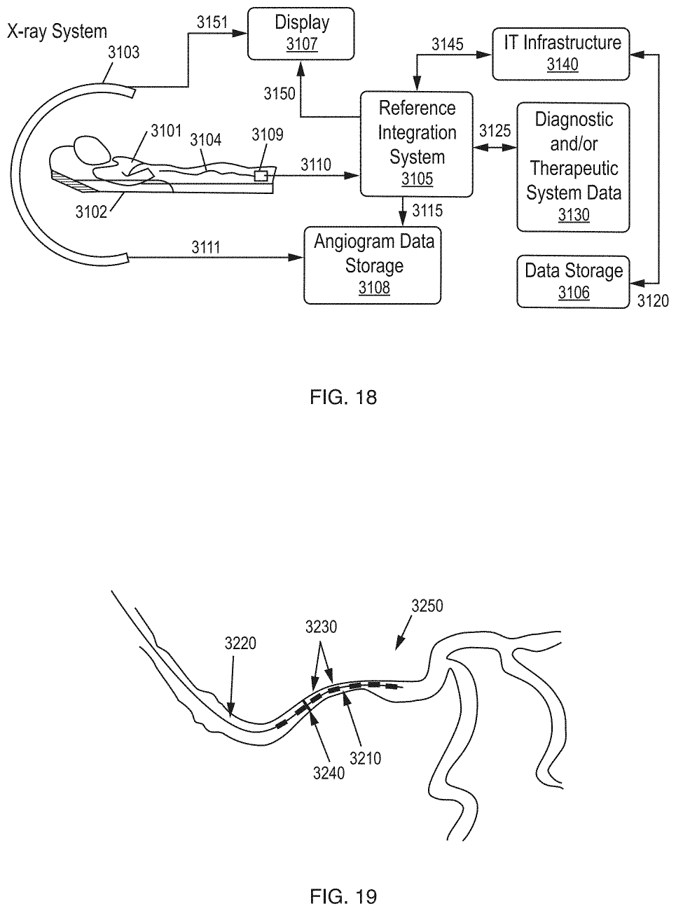

An optical light emitter/receiver 720 can include a small conical mirror 730 for reflecting light exiting from an optical fiber 735 disposed within the guidewire and can receive light and direct it into the optical fiber. Optical signal generation and receipt can be performed at a proximal end of the optical fiber, such as in a hub comprising a light source and sensor (see, e.g., hub 240 of FIG. 13A, FIG. 18, and FIG. 37). In the example illustrated in FIG. 7, a cone shaped reflection mirror 730 is mounted at the distal end of the fiber and can provide for 360-degree emission of light perpendicular to a length of the guidewire.

With a transducer mounted on or in a guidewire (either an ultrasound transducer or an optical transducer, either of which can act as both an emitter and a receiver), location registration with a diagnostic device can be performed in either an emission mode or a receiving mode, or a combination thereof. While operating in an emission mode, a signal emitted by the guidewire transducer can be detected by sensors of the diagnostic scanning catheter for location registration. While operating in a receiving mode, the guidewire transducer can capture signals emitted from the scanning catheter (e.g., either an acoustic or optical signal). A signal emitted by the guidewire transducer can be timed to provide for accurate location registration and to reduce interference to diagnostic signals.

FIG. 8 illustrates a simulation of a guidewire transducer emission signal on an axial cross-sectional view of an intravascular ultrasound image of a vessel generated during a diagnostic pullback scan.

IVUS imaging transducers can operate at a high pulse rate, normally 5000 Hz or higher. Within a single rotation of the imaging transducer, hundreds of pulses can be emitted. With each rotation, a signal received from each pulse is then composited by a processor to generate a single cross-sectional view of the vessel. As illustrated, a dark center hole 810 indicates a location of the catheter. A white section 820 indicates vessel tissue having more acoustic reflection, and a darker section 830 indicates an inner lumen of the vessel with blood or fluid and having less acoustic reflection. A boundary between these two areas indicates an inner surface of the vessel wall. Other features of a normal vessel, such as endothelium, intima, and adventitia, or disease features, such as calcium deposits, fibrotic lesions, and fatty lesions, can also be detected and measured by well-trained physicians.

A pulse signal 840 from a guidewire transducer emitting at a high rate can be visible within the frame, as detected when the imaging transducer is passing by (co-located with) the guidewire transducer. An acoustic wave travels in water and soft tissues at about 1,500,000 mm/s. The guidewire transducer can, for example, pulse at 1,500,000 Hz, and the pulse signal can be detectable on an IVUS image for every 1 mm of depth from a center of the image. An intravascular cross-sectional image with a 10 mm depth setting, for example, can show about 9-10 bright concentric curved white line segments, which can be easy to distinguish from normal fluid and tissue reflections. An emitter pulse rate of the guidewire transducer can be adapted to make the signal more, or less, densely clustered for ease of identification. A strength of the guidewire emission can also be adapted so as to appear detectable but not significantly interfere with reflected tissue signals from the IVUS sensor. A catheter displacement at which these frames are observed can be recorded as the displacement position at which the IVUS transducer is located next to the signal transducer on the guidewire.

FIGS. 9A and 9B show two examples of radiopaque markings for flexible elongate instruments. At least one marker of a plurality of markers can be independently distinguishable to provide for improved vessel location referencing between an X-ray angiographic display and a diagnostic pullback scan display. As illustrated in FIG. 9A, a flexible elongate instrument 910, such as a guidewire, includes five markers, with the second and fourth markers 930 having a visual appearance that is distinctive from the first, third, and fifth markers 940. The distinctive markers can provide for ease of visual correlation between the diagnostic image and X-ray angiography image without having to count markers. As illustrated in FIG. 9B, a flexible elongate instrument 920 includes three shorter markers 935 that can be used to provide a user with finer scaling and more accurate referencing at a middle portion of the wire. Having at least one uniquely identifiable imaging marker can be particularly helpful for measuring a sequence of imaging markers when not all of the plurality of imaging markers of the flexible elongate instrument are in the field of view of an X-ray image.

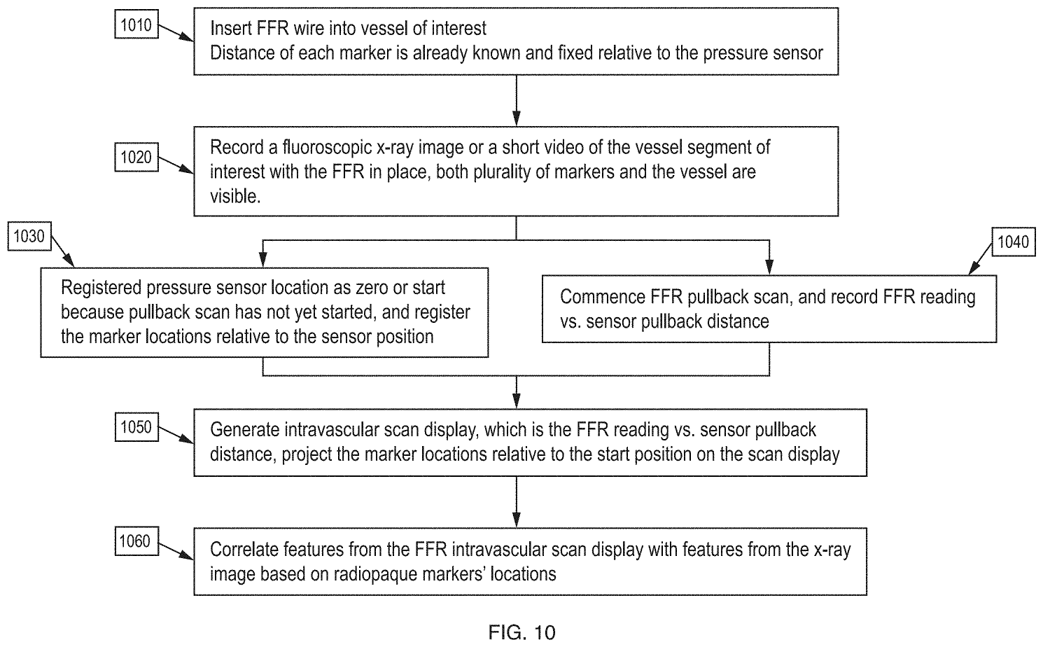

FIG. 10 is a flow chart of an example coronary intervention procedure involving an FFR wire having a plurality of markers, as shown in FIG. 4. An FFR diagnostic procedure begins with inserting the FFR wire into a coronary vessel of interest (1010), typically after normalizing a pressure output with the aortic pressure at a distal end of the guide catheter. The locations of the plurality of markers on the FFR wire disposed in the vessel are detected within an X-ray angiography image or video (1020). A location of the pressure sensor is registered with respect to imaging markers (1030), which can be executed by a processor automatically once an angiographic image or a short video of the markers inside the vessel have been detected. The FFR pullback scan commences and FFR readings versus sensor pullback distance are recorded (1040). Once the pullback scan is complete, a pullback display can be generated with the locations of the plurality of markers projected on the display (1050). For example, a composite image as shown in view 140b of FIG. 2B can be generated and displayed. Clinicians can use the displayed markers, located in both the X-ray angiography image or video and intravascular scan display to correlate vessel features using both imaging modalities (1060). The procedure shown in FIG. 10 can also be used for IVUS and OCT scans where markers are placed on an IVUS or OCT catheter or wire.

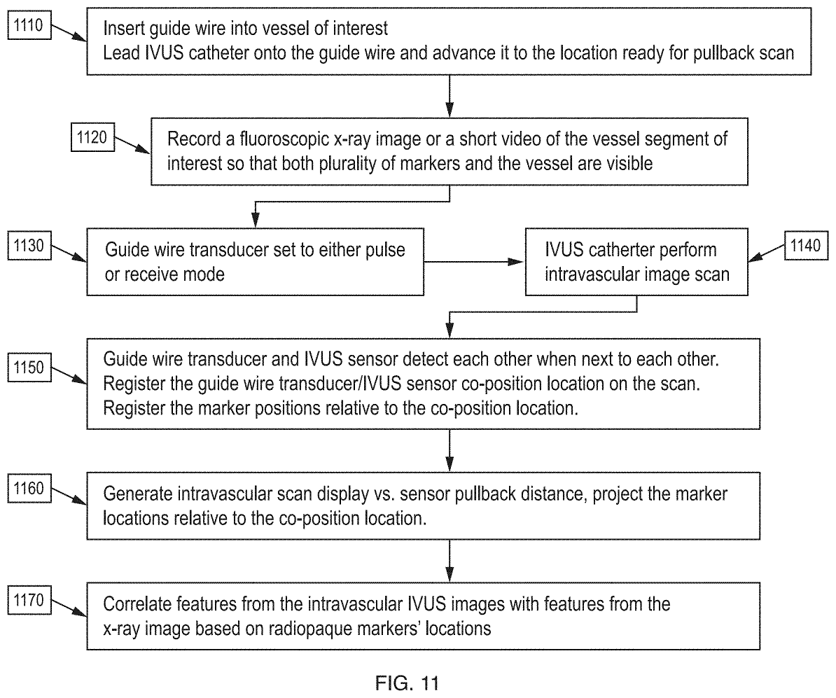

FIG. 11 is a flow chart of an example coronary intervention procedure involving guidewire having a plurality of markers and a signal emitter, as shown in FIG. 6, and an IVUS catheter. An IVUS diagnostic procedure begins with inserting the guidewire into the vessel of interest, followed by inserting the IVUS catheter over the wire to the vessel location (1110). The locations of the plurality of markers on the guidewire disposed in the vessel are detected within an X-ray angiography image or video (1120). The guidewire transducer can be set to either an emission mode or a receiving mode, depending on whether the guidewire is functionally connected to the IVUS system (1130), and the IVUS intravascular image scan is performed (1140). A position of the IVUS sensor is registered when the IVUS sensor is co-located with the guidewire transducer (1150).

An emission mode of the guidewire transducer can be used if the guidewire is not connected to the IVUS system. A pulse emitted by the guidewire transducer can be received by the IVUS sensor and displayed on an IVUS image, as shown in FIG. 8. Based on the IVUS images, a user can manually measure the IVUS sensor position that is closest to the guidewire transducer and input the location into the system. Alternatively, detection of the guidewire transducer pulse among the IVUS images can be automated and performed by a processor.

A receive mode of the guidewire transducer can be used if the guidewire is signally connected to the IVUS system. The emitted pulse by the IVUS sensor can be received by the guidewire transducer, from which the pullback location of the IVUS sensor that is closest to the guidewire transducer can be measured and automatically registered by the IVUS system. In this example, either the signal strength or timing, or both, can be used to calculate the position at which the IVUS sensor is closest to the guidewire transducer.

Once the position at which the IVUS sensor traverses the guidewire transducer has been measured, marker positions relative to the IVUS transducer can be determined as the marker positions relative to the guidewire transducer are known, and the marker positions can be projected on the IVUS pullback scan display (1160). For example, a composite image as shown in view 124b of FIG. 2B can be generated and displayed. Clinicians can use the displayed markers, located in both the X-ray angiography image or video and IVUS pullback scan display to correlate vessel features using both imaging modalities (1170).

For the projected locations of the imaging markers on a vessel diagnostic scan display to represent the same vessel locations as captured on an X-ray angiography image, a location of the starting point of a diagnostic scan (i.e., when the diagnostic sensor is at displacement point zero) can be quantified (referred to as “point zero location”) in reference to the plurality of markers as captured on the X-ray angiography image. For example, a first body lumen location can be quantified such that distances of each body lumen point where diagnostic data is collected can be determined with respect to the first body lumen location. The relationship can be expressed as follows: DR=FR+DF, where DR is a distance from a diagnostic sensor location to a reference point, FR is a distance from the first body lumen location to the reference point, and DF is a distance from the diagnostic sensor location to the first body lumen location.

DF can be expressed as follows: DF=OF+AD, where OF is a distance from the sensor starting point (the origin) to the first body lumen location and AD is an absolute displacement of the diagnostic sensor, starting from its origin at zero.

Combining the two equations provides for the following: DR=FR+OF+AD. The quantification of the point zero location can occur before, during, or after a diagnostic scan. For example, the point zero location can be determined from a detected co-location of the flexible elongate instruments (e.g., as described with respect to, for example, the devices of FIGS. 6-8). The displacement of a diagnostic sensor on a diagnostic instrument can be actuated and tracked by a motor drive unit disposed outside of a patient's body during a vessel scan. Alternatively, or in addition, sensor movement can be tracked by the X-ray equipment by continuously monitoring the position in the vessel. Alternatively, or in addition, sensor movement can be tracked by encodings disposed on a flexible elongate instrument, as described further in Sections 3 and 4 herein.

During a pullback intravascular scan, the pullback distance (i.e., the position of the transducer during a scan) can be continuously recorded. Such a displacement detection mechanism can be used when the diagnostic sensor and the plurality of imaging markers are disposed on the same flexible elongate endoluminal instrument. The positions of the imaging markers relative to the sensor are generally known before the X-ray angiography image of the vessel and imaging markers is obtained. This can be accomplished by the design of the flexible elongate endoluminal instrument. An example of such an implementation is a FFR wire comprising a plurality of radiopaque imaging markers positioned at the distal portion of the wire, near the pressure sensor. The relative positions of the plurality of imaging markers to the pressure sensor is therefore already known at the moment an X-ray angiography image of the vessel and markers is obtained. The markers can be projected on the longitudinal vessel scan such that their relative positions to the point where the X-ray angiography image is taken are the same as the physical imaging marker positions relative to the sensor on the flexible elongate endoluminal instrument. When displayed as such, the projected imaging marker positions on the vessel scan represent the marker positions relative to the vessel as detected by the X-ray angiography image. One disadvantage of using the pullback distance calculation as the displacement detection mechanism is that vessel features from the diagnostic scan can generally not be correlated to real-time X-ray angiography images because the flexible elongate instrument comprising the diagnostic instrument and the imaging markers have moved from the point where the X-ray angiography image was obtained. Furthermore, upon completion of an intravascular scan, the diagnostic scan instrument is typically removed from the patient, and other treatment devices, such as angioplasty balloons and stents, are inserted to treat vessel segment(s) that have been identified from the intravascular scan. It can be beneficial for the imaging markers to remain at the vessel location to help clinicians guide treatment devices to the vessel location of interest with or without real time X-ray angiography. As such, it can be beneficial to have the imaging markers disposed on an elongate instrument that can remain in place in the vessel throughout an entire PCT procedure, such as a guidewire.

Diagnostic instruments such as IVUS and OCT catheters can move along a guidewire that has been positioned inside the vessel. The guidewire does not need to move while other catheters move along it. After a diagnostic procedure, the guidewire can be left in place inside the vessel to be used by other catheter devices. When other devices are inserted and advanced along the guidewire, markers disposed on the guidewire can help clinicians guide the other devices to vessel features observed from intravascular scan. This is particularly useful because the markers are not only able to help guide another device to the vessel location on live X-ray, but also can help guide another device to locations displayed on a longitudinal vessel scan image by correlating its position from, for example, a live X-ray back to the intravascular scan image. In a situation that the guidewire position in the vessel has migrated, it can be easy to reposition it back to the position as the original X-ray capture image simply by using anatomical vessel landmarks such as branches.

When the positions of the imaging markers relative to a diagnostic sensor are not known, but can be measured from the obtained X-ray angiography image, displacement calculation mechanisms involving an initial measurement can be used. An example of such type of implementation is an IVUS catheter that is positioned in a vessel along a guidewire comprising a plurality of imaging markers affixed to its distal portion. A radiopaque imaging marker can be affixed near or at the IVUS transducer so that when the X-ray angiography image is obtained, the IVUS radiopaque imaging marker is also detectable on the X-ray angiogram image. Because the distance between each of the plurality of imaging markers and each imaging marker dimension of the guidewire is known, a relative position of the IVUS transducer to the plurality of imaging markers on the guidewire as recorded by the X-ray angiography image can be measured, either automatically using an imaging processing algorithm, or manually by a trained operator.

When the positions of the imaging markers relative to a diagnostic sensor are not known, but can be measured during the intravascular pullback scan, displacement calculation mechanisms involving at least one co-location determination can be used. Diagnostic instruments such as IVUS and OCT include, respectively, ultrasound and optical sensors for vessel diagnostics. An ultrasound sensor made of piezoelectric material can function as both a signal emitter and receiver. An optical sensor using a fiber optic cable can also be configured to function both as an emitter and receiver. For the purpose of this description, both IVUS and OCT sensors are referred to as diagnostic sensors. A signal transducer can be adapted to be mounted on a guidewire at a location near or within the guidewire segment that comprises the plurality of imaging markers. With this adaptation, a location of the guidewire-mounted signal transducer relative to the markers is known and fixed. Signals from the guidewire-mounted transducer and diagnostic sensor can register when the sensors are aligned next to each other at a position in a vessel. A signal emitted by the guidewire-mounted transducer can be received by the diagnostic sensor and may also be displayed on the vessel pullback scan. The location on the pullback scan where the signal is received can be determined to be location at which the guidewire-mounted transducer and diagnostic sensor are aligned. If a guidewire-based signal transducer is connected to the diagnostic instrument system, the alignment positions can be even more accurately measured by an imaging processor. Both signal pattern and timing can be used to measure the alignment position. At the point when guidewire transducer is aligned with the diagnostic sensor, the guide-wire marker locations relative to the diagnostic sensor can be measured. Because the diagnostic sensor scan distance is tracked, the positions of the plurality of markers relative to the sensor at the moment when the X-ray angiography image was taken can therefore be calculated from the traveled distance of the diagnostic sensor.

### 2. ENCODING METHODS AND SYSTEMS

Body lumen diagnostic modalities often require that a diagnostic device scan through a body lumen length, generating body lumen information at closely spaced displacement points. In most currently available systems, a displacement of the diagnostic device is actuated by a motor drive unit placed outside of a patient, and the tracking of displacement also occurs outside of the body lumen. There can be a large discrepancy between the measured displacement of a diagnostic device as estimated by a motor drive unit and an actual sensor displacement inside of the body lumen. Discrepancies can result due to diameter differences between a moving medical instrument and a guide catheter and the effects of inherent vessel elasticity. Precise length measurement of vessel features can be needed to properly choose a size of a treatment device (e.g., an angioplasty balloon, cutting balloon, and stents). While constant X-ray angiography can be used to track the movement of a diagnostic sensor displacement, this method exposes the patient and/or operator to high levels of X-ray radiation.

Once a vessel endoluminal diagnostic procedure has been performed that provides more detailed information about the vessel lumen than from an X-ray angiography, a treatment decision is often made based on the endoluminal diagnostic information. The treatment decision can be based on a precise location within the vessel of the lesion. Typically, subsequent treatment procedures are guided by X-ray imaging alone. Even with the benefit of vessel location correlation between an X-ray image and endoluminal diagnostic images, it can be desirable that the location of a treatment device moving inside of a vessel lumen be visualized directly in real-time or about real-time on an endoluminal diagnostic image previously generated to help position the diagnostic and/or therapeutic device at a vessel location of interest that has been identified on the diagnostic image. In some instances, a clinician can use features that are visible on both an X-ray and endoluminal diagnostic scan, such as a vessel branch or severe narrowing, to help identify corresponding locations so as to attempt to improve the measurement accuracy of a guided therapeutic and/or diagnostic device during a PCI.

When performing an endoluminal diagnostic scan, the diagnostic sensor receives vessel specific information (e.g., vessel size, tissue morphology, pressure, temperature, etc.) while moving longitudinally within a vessel segment, and the acquired vessel information data and the sensor displacements are both recorded and correlated.

The resulting data set comprises paired data of displacement points and the collected vessel information at each of the displacement points (e.g., as shown in FIG. 2A). The data set is output to a processor and can be displayed on a screen in numerical and/or representative graphical forms.

While standard X-ray angiogram imaging presents a 2-D projection of the blood vessel from outside of the vessel, intravascular assessment modalities assess a vessel from within the vessel lumen, and can generate vessel lumen information at many thousands of displacement points during a diagnostic scan.

To provide for more accurate location correlation among an X-ray angiogram and a diagnostic and/or therapeutic device position, systems and methods providing for more precise device tracking within a vessel are described.

For example, a location information sensor (e.g., sensor 120, 122) can be disposed on one of two flexible elongate instruments and configured to detect encoding markers disposed on the other of the two flexible elongate instruments. The encoding markers can be disposed at and detected at a distal portion of the flexible elongate instruments to provide for accuracy at the location of interest within a vessel. One of the two flexible elongate instruments can further include imaging markers to provide for correlation to an X-ray image.

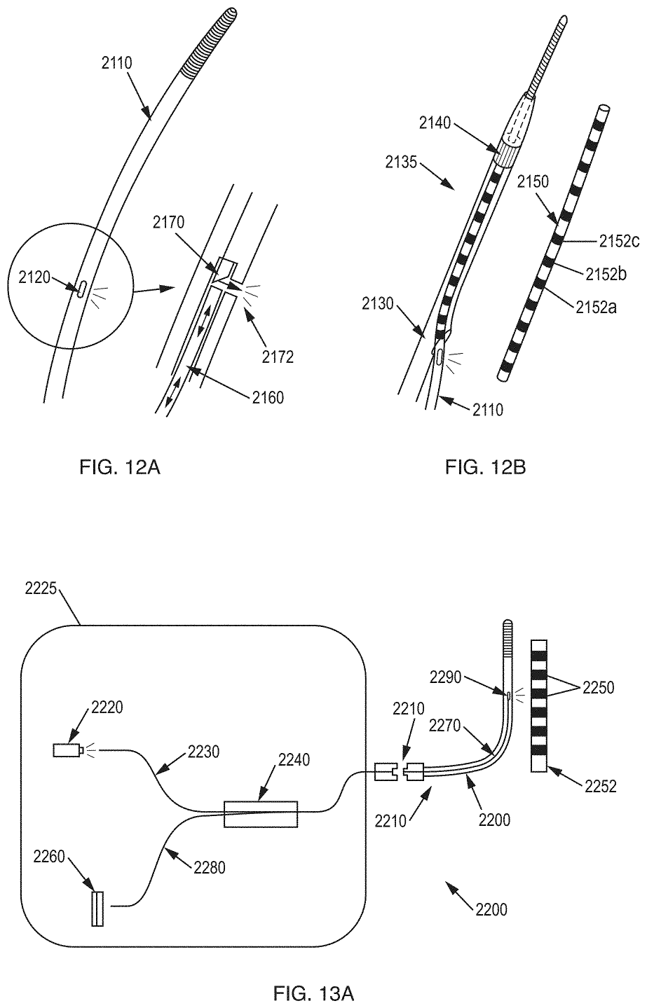

As illustrated in FIG. 12A, a first flexible elongate instrument 2110 can be a guidewire (guidewire imaging markers not shown in FIG. 12 for clarity) with an optical encoding sensor 2120 mounted at the distal portion of the flexible elongate instrument. The guidewire is used in conjunction with a second flexible elongate instrument 2130 which, as illustrated in FIG. 12B, is a phased array IVUS catheter that can generate body lumen morphology information when inserted in a body lumen. However, any catheter can be configured to be used with such a guidewire such that a displacement of the catheter relative to the guidewire can be measured and output to a processor/computer. The displacement information can be correlated to diagnostic body lumen information obtained at each diagnostic point. The phased array IVUS catheter 2130 includes a phased array acoustic transducer 2140 affixed near its distal tip. The catheter includes portions with one or a plurality of displacement encoding markers and portions without displacement encoding markers, which can optionally be configured to be in a periodic order. A monorail portion 2135 of the IVUS catheter includes a liner 2150 that is marked with optical linear encoding. The liner 2150 can be disposed within the monorail portion of the IVUS catheter, such that, as the catheter traverses over the guidewire 2110, the optical encodings are detected by the sensor 2120.

As further illustrated in FIG. 12A, a displacement signal can be transmitted through an optical fiber 2160. The guidewire 2110 can include, for example, a 45-degree polished fiber termination 2170 with a reflective coating configured to divert light from the fiber towards an aperture 2172 and to divert light reflected back to the aperture 2172 from the encoding markers, down the optical fiber to a light intensity meter. The encoding sensor 2120 detects an encoding signal from the inner diameter surface of the monorail liner and sends the signal to a signal processor for conversion to displacement information. In a simplified example implementation, when there is relative movement between the guidewire and the catheter, the optical encoding sensor can detect changes in reflected light intensity due to encoding markings of different reflectance at specified intervals. A processor (e.g., processor 105, alternatively referred to as a calculation unit) can be configured to count changes in signal intensity, from which a displacement between the IVUS catheter and the guidewire can be established.

Optionally, each of the displacement encoding markers 2152a-c can comprise a different color (e.g., red, green, and blue (RGB)) or a different greyscale intensity, with white light illumination from the optical transducer 2120, and an RGB-sensitive or greyscale-sensitive detector (e.g., sensor 2260, FIG. 13). Such an implementation has the advantage of providing different reflected signal time patterns, which can enable automatic direction detection.

The catheter 2130 can be displaced at constant velocity, whereby both the distance/time between each encoding marker and/or the reflectance of a selected encoding marker can be used for labeled displacement detection. In such an implementation, a displacement from a start location can be labeled in conjunction with the encoding, thereby eliminating a need to count a specific number of encoding markers to measure a displacement distance between the sensor and the flexible elongate instrument that includes the displacement encoding markers.

In the above examples, the encoding sensor is disposed on or in a guidewire (as a first flexible elongate instrument) and the encoding markers are disposed on or in the catheter guidewire lumen liner within a catheter (as a second flexible elongate instrument). However, the positioning of the encoding sensor and encoding markers can vary. For example, because movement between the two flexible elongate instruments is relative, an equivalent measurement can be obtained if the guidewire is configured to provide the linear encoding and the catheter is configured to include an optical sensor with which an encoding signal can be detected.

FIGS. 13A-C show another example system 2200 that includes an optical-fiber-based linear encoding and an encoding detector. A light beam can be input into an optical fiber 2270 built into or onto a flexible elongate instrument 2200, such as a guidewire or any catheter based device, via an optional detachable connection 2210 at its proximal end 2212. The light beam can originate from a light source 2220 disposed external of the body. Components of the system that can remain external of the body are indicated by 2225, which can advantageously provide for the flexible elongate instrument to maintain a minimal profile for insertion. Light from the light source 2200 can be transmitted via an optical fiber 2230, into a light splitter 2240, to the detachable connection 2210, and into the instrument 2200. The light can be projected out of the fiber 2270 at the optical encoding sensor aperture 2290, and light reflecting from the encoding markings 2250 of the catheter or catheter liner 2252 can enter back into the fiber 2270, be transmitted back through the light splitter 2240, through fiber 2280, and to a light intensity meter 2260. A change in intensity due to relative movement between the optical reader 2290 and the encoding markings 2250 can be tracked by a signal processer (e.g., processor 105), as illustrated in graph 2205 of FIG. 13B, and translated into relative displacement between the guidewire and the catheter devices, as illustrated in graph 2215 in FIG. 13B. Optionally, a transducer can include a light source. The light source can be, for example, a laser or a light emitting diode. Optionally, the light source can instead be positioned within the guidewire. The light source can be monochromatic of a preferred wave length, or multi-wavelength, depending on the encoding. Longer wavelengths in the infrared range can be less impacted by potential contamination, such as by blood or other body fluids.

The reflected light from the encoding markings 2250 can be transmitted back through fiber 2270 and split by light splitter 2240. At least a portion of the reflected light is delivered to a light intensity meter 2260 through the fiber 2280.

Optical fibers can be obtained with a 50-micron core with overall diameter of 65 microns (Polymicro Technologies (Phoenix, Ariz.), which is sufficiently small to be positioned inside a guidewire or a catheter device. An optical fiber disposed within or on a flexible elongate instrument can be of a diameter ranging from about 20 microns to about 1000 microns.

The emitted light can be continuous or rapidly pulsed so as to not develop aliasing during fast movement. The encoding on instrument 2252 can include two regions of reflectance, as illustrated with encoding markers 2250 in FIG. 13A. In some regions, the two regions of reflectance can be black/white, red/blue, green/red, black/grey, or blue/green, for example. While FIG. 13A illustrates a guidewire 2200 as having an integrated optical fiber and a catheter portion having encoding markers, the displacement encoding markers can instead be configured to be on an outer diameter of the guidewire, and the encoding sensor can be located on an inner diameter of the catheter guidewire lumen, or vice versa. The multiple available variations on relative positions of encoding markers and sensors provides flexibility where one flexible elongate instrument is unable to provide for an optical fiber passage.

A measured reflected intensity signal over time from reflectance encoding markers can be binary, as shown in the example graph 2205 of FIG. 13B. A processor can count the peaks and valleys (e.g., second derivative, positive or negative, respectively) to measure a distance that the optical reader has traveled along the encoded surface. Displacement over time can be calculated and/or displayed, as shown in example graph 2215 of FIG. 13B.

As illustrated in FIG. 13A, the encoding markers appear at a consistent density along a length of the instrument 2252. However, an encoding can comprise a plurality of regions in which each region has a different density of encoding markers, for example, at a distal or proximal region. Alternatively, or in addition, encodings can comprise markers of varying densities to provide for an indication of direction.

A three-reflectance encoding is shown on instrument 2225 in FIG. 13C. As illustrated markers 2226, 2227, and 2228 are of different greyscale density. The reflected light intensity signal over time from the three-reflectance encoding in one direction is shown in the example graph 2235. If the instruments travel in an opposite direction, the shape of the graph 2235 is reversed. The three-reflectance encoding can advantageously provide for directional information of the relative movement between a guidewire and a catheter. A user therefore does not need to manually input a direction of travel at the start of a displacement process.

Encoding markings can be positioned on either an outer diameter surface or an inner diameter surface of a flexible elongate instrument. Optionally, displacement encoding markers on an outer diameter surface can comprise a first pigment of a selected reflectance, and encoding markers on an inner diameter surface can comprise a second pigment of a different selected reflectance. The different reflectance pigments can result in different reflectivity profiles.

The displacement encoding markers can comprise a laser engraving such that micro-grooves of different depths are provided on the encoding surface. For example, a deeper groove can result in a decreased reflection intensity as compared with a shallower groove.

One option for creating an encoding marker is with use of a laser to remove a dark oxidation layer on a metal surface that has been anodized. Another option for creating an encoding marker is to paint an encoding surface with rings of different pigments (e.g., red, green, and blue. The displacement encoding markers and encoding sensor can be based on optical, capacitive, inductive, resistive, electromagnetic, piezoelectric, or magnetic properties.

Generally, due to the small clearance between an outer diameter of the guidewire and an inner diameter of a catheter guidewire lumen, which is typically less than 50 microns, contamination of the encoding surface or light reader by blood is of minimal concern.

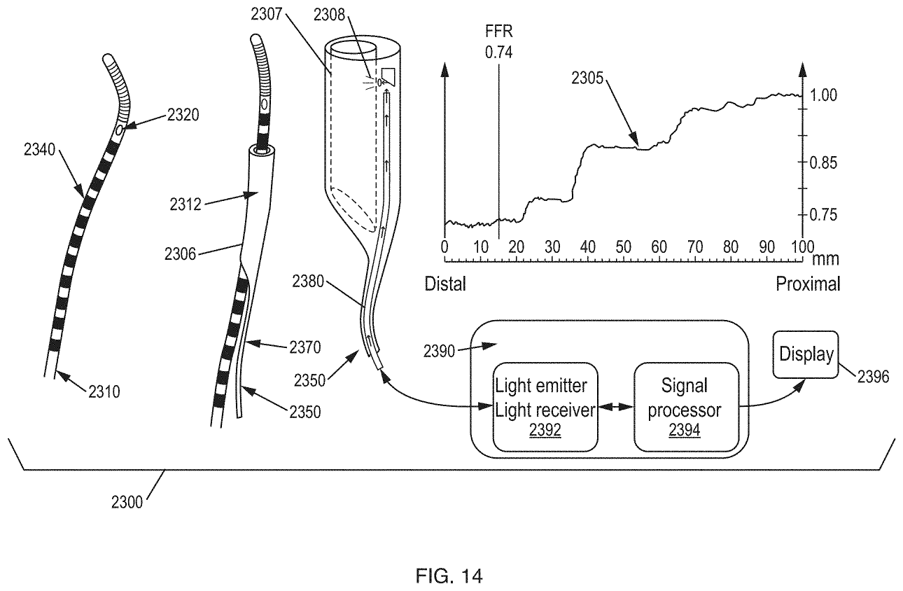

FIG. 14 depicts another example system 2300 that includes an optical-fiber-based linear encoding and an encoding detector. As illustrated, a first flexible elongate instrument 2310 is an FFR wire with a blood pressure sensor 2320 at the distal portion of the device and a section that is marked with optical encoding 2340. Radiopaque markings can also be included on the instrument 2310 (not shown in FIG. 14 for clarity). A second flexible elongate instrument 2350 includes an encoding reading catheter 2306, with an optical encoding sensor 2308 mounted at an inner surface of its guidewire lumen 2307 and facing the guidewire when the guidewire is inserted.

The reading catheter 2306 can be constructed with a short and low profile over-the-wire section 2312 to minimize interruption to blood flow, and a long shaft section 2370 that contains an optical fiber 2380, which is connected to a subsystem 2390 that includes a light emitter and light intensity meter 2392 and a signal processer 2394. The system 2300 can further include a display 2396 configured to display FFR ratio versus displacement distance, as shown in graph 2305.

The FFR wire 2310 can first be inserted into a coronary vessel and advanced to a location of interest. The reading micro-catheter 2306 can then be inserted over the FFR wire and follow the FFR wire until the encoding sensor 2308 reaches the region comprising encoding markers 2340 on the FFR wire near the location of interest. The micro-catheter can be held stationary relative to the vessel. During an FFR diagnostic vessel scan, the FFR wire is pulled back in the coronary vessel while obtaining blood pressure readings, and the encoding sensor provides an encoding signal to the signal processor, which translates the encoding signal to distance displacement. For example, the reading catheter can be held stationary at a coronary vessel location that is just proximal of the coronary ostium, which can provide for minimal disturbance to coronary blood flow.

In conventional methods, FFR pullback distance is measured by either a motor drive unit outside of the body or tracked by X-ray angiography to continuously monitor movement of the FFR wire. Placing the motor drive unit outside of the body can result in large measurement discrepancies due to wire movement slacks caused by the size difference between an FFR wire and a guide catheter and/or multiple tortuous turns, and due to the long path before the device reaches the coronary vessels. Tracking FFR wire movement using continuous X-ray angiography can result in significant X-ray radiation doses to the clinician and/or patient. Typically, FFR procedures require that a pullback speed not be too fast because of the need for averaging heart beat pressure for accurate FFR value determination. To obtain accurate FFR values with sub 1 mm pullback distance resolution, for example, a wire pullback speed is limited to about 1 mm/sec if the patient's heart rate is 60 beats/sec. At 1 mm/sec, a 90 mm pullback distance takes 90 seconds to complete, which equates to 90 seconds of continuous X-ray exposure.

Systems and methods described herein can enable clinicians to obtain accurate FFR pressure measurements at precise locations with high pullback distance resolution without concern for excessive X-ray radiation exposure. The methods described can also provide for re-advancing the FFR wire back to re-assess readings at any vessel points of interest while maintaining an accurate pullback distance measurement. For example, the FFR wire can be pushed (rather than pulled), and the displacement measured using the encoding markers can provide for accurate location information.

FIG. 15 illustrates an example system 2400 providing for location determination of a therapeutic device. As illustrated, a first flexible elongate instrument 2430 is a guidewire having a plurality of radiopaque imaging markers 2460 positioned in a vessel lumen 2420 at a location of interest. A second flexible elongate instrument 2410 is catheter on which an angioplasty balloon 2400 is mounted.

A length of each of the radiopaque imaging markers 2460 and the distances between each of the imaging markers is known. A location of the angioplasty balloon 2400 can be measured relative to the vessel markings 2440 in a depiction of an angiographic X-ray image 2450 of the vessel lumen capturing the plurality of radiopaque markers 2460. The angiographic image 2450 need not be a real-time image, and the X-ray imager does not need to be on and emitting X-rays to determine a location of the balloon 2400 with respect to the image 2450. The angiographic image 2450 can be obtained with the guidewire 2430 inserted in the blood vessel 2420 such that both the plurality of imaging markers 2460 and the blood vessel 2420 can be identified in the image. Optionally, a plurality of X-ray angiographic body lumen images can be obtained from different angles, with the guidewire remaining at the same body lumen location, which can advantageously provide for 3D modelling of the vessel and instruments within the vessel, as described further below.

The angioplasty balloon catheter 2410 includes optical encoding 2470 positioned proximal to the balloon at a selected distance. An encoding sensor 2480 is affixed to or included in the guidewire, which is at a selected distance from the plurality of imaging markers 2460. A relative position between the angioplasty balloon on the catheter and the plurality of markers on the guidewire can therefore be known when the encoding sensor 2480 first engages with the encoding 2470 on the angioplasty catheter. This position is referred to as the first engagement position, as shown in the figure. The short line 2490 appearing in the x-ray image 2450 depicts the location of the distal end of the balloon when the balloon catheter is at the engagement position with the guidewire. Once an angiographic image of the vessel is obtained with the positions of the plurality of imaging markers along the vessel identified in the image, a location of the angioplasty balloon in the vessel at the first engagement position can be measured. The vessel location of the angioplasty balloon can be continuously measurable thereafter, provided the encoding sensor remains within the encoded region of the angioplasty balloon catheter. The location of the angioplasty balloon in the vessel can be displayed in real-time in a linear fashion as shown by display 2415, for example, in which a simulated depiction of the vessel markings 2440 appear as markings 2425 and a simulated depiction of the balloon 2400 appears as balloon 2435. The representation of the balloon 2400 can be dimensionally scaled with respect to the vessel lumber to represent a true indication of its overall position.

If the encoding sensor 2480 moves out of range of the encoding 2470, a location of the balloon can be re-acquired when the encoding sensor re-engages with the encoded region. The balloon can stay within the length of the plurality of radiopaque imaging markers when the encoding sensor is within the length of the encoded region to maximize a range that the plurality of imaging markers can provide as an aid for vessel location correlation.

When tracking and displaying the angioplasty balloon location relative to the position of the plurality of imaging markings is performed in real-time or about real-time, the imaging markings can be used to correlate the balloon position in the vessel image in the angiography for its navigation rather than using real time X-ray imaging to reduce radiation exposure.

FIG. 16 illustrates an example display 2500 using the system of FIG. 15 to provide real-time guidance of an angioplasty balloon when moving inside a vessel to an identified vessel narrowing location 2530. A real-time location of a balloon, represented by simulated balloon 2540, is displayed in a previously obtained diagnostic IVUS scan image 2520 using an IVUS catheter and a guidewire arrangement.

In the example shown in FIG. 16, the first flexible elongate instrument is an IVUS catheter that includes one or a plurality of displacement encoding markers located at a selected distance from the ultrasound transducer (e.g., as shown in FIG. 12B). The second flexible elongate instrument is a guidewire that includes a plurality of radiopaque imaging markers that are detectable by X-ray angiography and an encoding sensor positioned at a selected distance to the plurality of markers (e.g., as shown in FIG. 12A). The imaging marker locations 2550 detected in the X-ray angiographic image of the vessel 2510 with the inserted guidewire can also be projected in an IVUS scan image 2520 and shown as simulated markings 2570.

In this example, the IVUS diagnostic procedure can provide for identification of a vessel narrowing location 2530, on which a treatment decision can be based for the placement of an angioplasty balloon. After the IVUS procedure, the IVUS catheter can be removed, while the guidewire with the plurality of radiopaque imaging markers is left in position in the vessel. An angioplasty balloon can then be inserted and advanced into the vessel via the same guidewire. Once the angioplasty balloon catheter is advanced to a first engagement position, the location of the balloon can be measured, and a simulated representation of the balloon 2540 can be projected in real-time onto the previously obtained IVUS scan image on the displacement axis. A length of the simulated balloon 2540 can be based on the length of the actual balloon used.

As the balloon advances distally within the vessel, the real-time display can show that the simulated balloon is moving from right to left in the IVUS scan image, towards the narrowing 2530. In the diagnostic IVUS scan image shown here, the distal end of the balloon is near the second marking, which correlates to position 2560 in the angiographic vessel image. The display can further include an indication within or projected onto the angiographic vessel image 2510 or the balloon position 2560.

Optionally, multiple angiographic images can be displayed along with the diagnostic IVUS scan image to provide different angle of views of the vessel location when multiple angiographic images from different angles of view are obtained.

Advanced rendering of the vessel lumen, including, for example, a 3D display and/or an internal lumen view display can be generated based on either the vessel lumen diagnostic scan image and/or X-ray angiographic images. The locations of the imaging markings and the location of a diagnostic device can be projected in such a display.

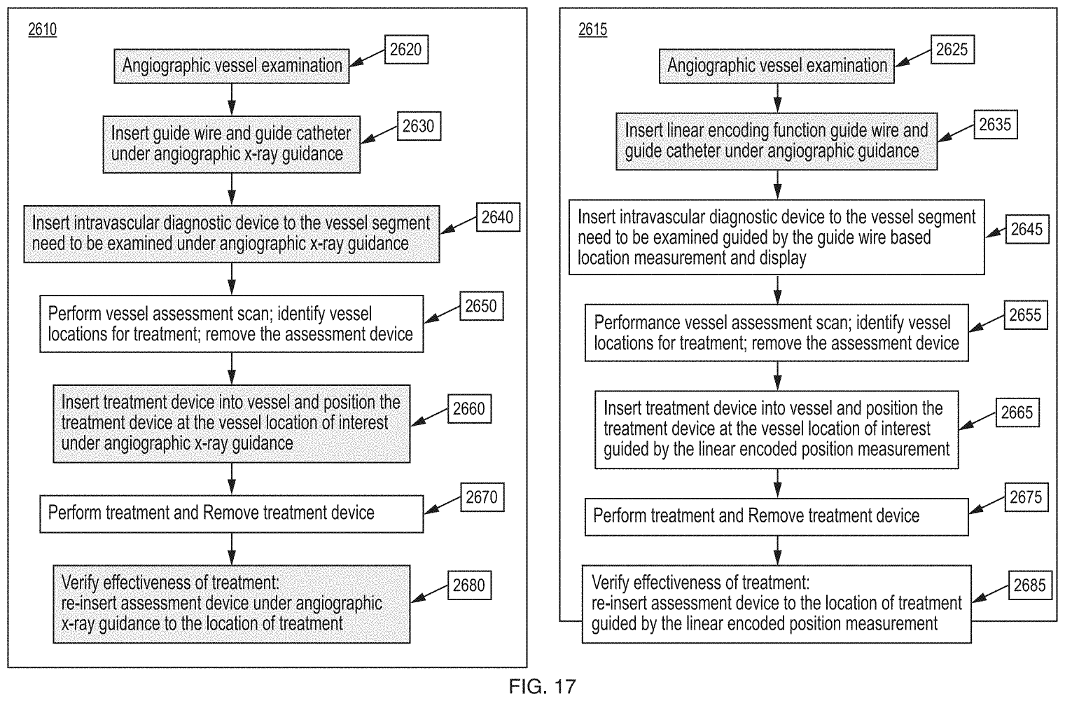

FIG. 17 is a flow diagram depicting a method 2610 that includes a standard therapeutic delivery process involving live, X-ray-guided device navigation alongside a method 2615 of delivering a therapy with marker-guided device navigation using systems as shown in, for example, FIGS. 11-16. In a standard therapeutic delivery work flow for non-complication IVUS and/or OCT-guided PCI procedures, X-ray imaging is used as the primary means for intravascular guidance. In contrast, a work flow using the devices described herein can be performed without X-ray imaging after an initial angiographic vessel examination 2625 and guidewire insertion 2635 are performed. The gray shaded boxes in FIG. 18 indicate steps that involve X-ray angiographic guidance and contrast agent injection. As visible in the figure, out of the five steps involving X-ray and contrast agent injection in the current standard work flow 2610 (i.e., steps 2620, 2630, 2640, 2660, and 2680), three can be replaced by the endoluminal device based guidance methods described herein (i.e., steps 2640, 2660, and 2680), thereby reducing the number of procedure steps involving X-ray exposure from five to two.

Currently, in a standard PCI work flow, an angiographic examination of the vessel is performed to identify the coronary branch that needs intervention (2620), followed by insertion of a guidewire into the identified coronary branch (2630). Both procedure steps (2620, 2630) are performed under angiographic X-ray imaging and guidance. Once the guidewire is in place, a diagnostic device (e.g., IVUS, OCT, and/or FFR device) is inserted for a more detailed examination of the vessel. The insertion and placement of the diagnostic device (2640) is performed using real-time X-ray guidance. Once the diagnostic device is positioned in a desired vessel location, a vessel lumen diagnostic scan can be performed (2650) and a vessel location for treatment can be measured. The diagnostic device is then removed from the vessel so that a treatment device can be inserted on the guidewire.

The insertion and placement of a treatment device (2660) is guided by real-time X-ray angiography to the identified vessel location. Without being able to correlate the vessel location identified from the diagnostic scan image to the angiographic X-ray vessel image, the targeted vessel location in the X-ray image is estimated. The accuracy of placing the therapeutic device to a selected location is highly dependent on the experience and training of the clinician. Once the therapeutic device is in the target location, standard treatment procedures such as balloon inflation, stent expansion, etc. can be performed (2670) without X-ray angiography. After performing the treatment, a diagnostic device may be optionally re-inserted to verify the effectiveness of the treatment (2680).

In contrast, in a guidewire-based location measurement method, after an angiographic vessel examination (2625) and insertion of a guidewire (2635), treatment device guidance can be performed with the X-ray turned off. As illustrated in FIG. 17, insertion of an intravascular diagnostic device (2645), vessel assessment (2655), insertion of a treatment device (2665), treatment (2675), and, optionally, reinsertion of a diagnostic device to verify treatment effectiveness (2685) can be performed without X-ray. Furthermore, the vessel location of the treatment can be projected onto the vessel diagnostic scan image from which the target site was identified, providing for an estimation of the location in the angiographic X-ray image, resulting in a more precise therapeutic device placement in the vessel. Further still, the guidewire-based location measurement method of this disclosure can guide the diagnostic device back to the treated target vessel site without using the X-ray instrument to verify treatment. Optionally, a brief X-ray angiography can be used to verify treatment device location accuracy. The X-ray emission time, however, can be minimal and the verification can be performed without additional contrast injection because the imaging markers are detectable in the X-ray image.

### 3. MULTI-MODALITY IMAGING CO-LOCATION SYSTEMS AND METHODS

To optimize clinical decisions and outcomes for intravascular intervention in an effective, efficient, safe, and cost sensitive manner, a precise, real-time position detection based on co-location can be performed among multiple diagnostic and therapeutic devices and systems during a complex percutaneous intervention procedure. Methods, systems and workflows for such guided procedures are described herein.

Intravascular intervention methods that yield better clinical decisions, outcomes, and safety for the operator and/or patient are provided. A flexible elongate instrument equipped with position sensing can provide real time position co-location of a guidewire and a diagnostic or therapeutic device. The methods described herein enable an operator (e.g., technician or physician) to obtain real-time or about real-time image customization and flexibility during each procedure step while minimizing radiation exposure.

The methods and systems provided are described below by way of examples involving an endoluminal device, a flexible elongate instrument with a plurality of imaging markers of known spacing and dimension to serve as a reference for device position(s), and a same or different flexible elongate instrument with displacement sensing capability. The examples are described within the context of an interventional cardiology catheterization laboratory. The data acquisition and processing, location detection, communication and real-time co-location among multiple modality displays are described by way of example via X-ray angiographic images, diagnostic intravascular images, such as IVUS and OCT, physiology probes, such as FFR and iFR, and therapeutic devices, such as balloon catheters and stents, that can be mounted upon a guidewire (e.g., guidewire 110, 2110, 2210, 2310).

Co-location information can be obtained with respect to the position of a second flexible elongate instrument (e.g., a therapeutic and/or diagnostic device) in relation to a first flexible elongate instrument (e.g., a guidewire) through the following method, which includes use of a therapeutic and/or diagnostic device that includes a transducer (e.g., ultra-sound or light, emitter and detector) and a flexible elongate instrument that includes a plurality of radiopaque markers and a transducer or sensor: (a) the first flexible elongate instrument is first inserted into the body lumen, (b) the therapeutic and/or diagnostic device, or a catheter comprising the therapeutic and/or diagnostic device, is inserted into the body lumen using the flexible elongate instrument as a guidewire, (c) the transducer of the therapeutic and/or diagnostic device traverses past the plurality of radiopaque markers either in push-mode or pullback mode, (d) the transducer of the therapeutic and/or diagnostic device emits a signal (e.g., ultra-sound or light), (e) the transducer or sensor of the first flexible elongate instrument detects the signal emitted from the transducer of the therapeutic and/or diagnostic device, (f) the transducer or sensor of the first flexible elongate instrument sends a signal to the calculation unit, the signal including information regarding the time, intensity, and/or pattern of the detected signal, and (g) the calculation unit compares the signal sent from the transducer to expected signal information for a pre-selected position of the therapeutic and/or diagnostic device in relation to at least one of the radiopaque markers on the first flexible elongate instrument. Optionally, the method further includes: (h) the calculation unit receives information from a secondary imaging method of the body lumen (which can include or exclude X-ray imaging), (i) the calculation unit superimposes the expected relative position of the therapeutic and/or diagnostic device and/or the flexible elongate instrument with the body lumen, (j) the calculation unit sends a signal to a display for the superimposed expected relative position of the therapeutic and/or diagnostic device and/or the flexible elongate instrument within the body lumen. Obtaining co-location information regarding the position of the therapeutic and/or diagnostic device in relation to the first flexible elongate instrument can be repeated at selected intervals (e.g., at each unit of displacement during pullback or push-through).

Reference is now made to FIG. 18, which illustrates an example system for carrying out the provided methods in the form of a Reference Integration System 3105 and associated apparatuses used in a catheterization laboratory. The Reference Integration System 3105 can include subsystems for any of: (1) receiving real-time or about real-time angiographic information with a flexible elongate instrument disposed inside a patient, and processing the angiographic information to establish 2D and/or 3D models of the plurality of radiopaque markers on the flexible elongate instrument for superimposing on corresponding lumen images; (2) receiving real-time or about real-time position and/or displacement information for a therapeutic/diagnostic device from a Device Position Acquisition System (e.g., a system comprising a sensor and labelling markers disposed on a first and/or second flexible elongate instrument, such as system 100), integrating the position/displacement information with the 2D and/or 3D models of the plurality of radiopaque markers, generating a real-time or about real-time device position illustration and superimposition with the 2D and/or 3D model, generating a position correlation display via real-time or about real-time data integration among the radiopaque marker 2D/3D model and any of: corresponding lumen image(s), simulated device illustration(s), diagnostic and therapeutic system data, and angiogram data; (3) providing data storage for X-ray imaging, Device Position Acquisition System data, modeling data, and position correlation display data; and, (4) providing bi-directional data communication with a body imaging system (e.g., an X-ray angiography system), a therapeutic and/or diagnostic system, a sensor, data storage, display, operator/physician interface, and local and/or external computer network systems.

As illustrated in FIG. 18, a patient 3101 is positioned upon an angiographic table 3102. The angiographic table 3102 is arranged to provide sufficient space for the positioning of an X-ray system 3103 (e.g., including angiography/fluoroscopy equipment) set-up in an operative position in relation to the patient 3101 on the table 3102. X-ray imaging data can be acquired by the X-ray system 3103 with presence of contrast flow in the patient's blood vessels at various projections to assess lesions of interest. A guidewire 3104 is inserted into a lumen of the patient 3101 (e.g. a blood vessel, such as a coronary artery). The X-ray system 3103 acquires real-time or about real-time fluoroscopic images of the guidewire in the absence of contrast flow at the targeted blood vessel(s) during insertion of the guidewire. A final position of the guidewire 3104, corresponding to a lesion of interest 3210 (FIG. 19), is measured. With the guidewire inside the vessel, one or a plurality of angiographic and/or fluoroscopic images from one or more angiogram projections can be taken with and/or without contrast flow. The angiographic/fluoroscopic images are archived in an Angiogram Data Storage 3108, which is connected via interface 3111 to a local data storage 3106 inside the catheterization lab, optimally in various formats, such as native binary and DICOM formats, as selected by the users. The therapeutic and/or diagnostic system in this example comprises the guidewire 3104, which can be equipped with a linear encoding reader at its distal end to measure device displacement (e.g., sensor 120, 2120, 2290), an electronic device referred to as a Hub 3109 that is attached at a proximal end, of the guidewire and which converts the device displacement signal to an electrical signal, and an interface 3110 to the Reference Integration System 3105. The therapeutic and/or diagnostic device positioning data acquired by the guidewire 3104 are streamed to the Reference Integration System 3105 via the interface 3110 from the Hub 3109.

The displacement of the therapeutic and/or diagnostic device inside the lumen can be measured via the linear encoding reader of the guidewire. Alternatively, or in addition, the displacement of the therapeutic and/or diagnostic device inside the lumen can be measured via the diagnostic and/or therapeutic system (e.g., through a pullback sensor 126). The displacement data is part of the Diagnostic and/or Therapeutic System Data 3130, connected via interface 3125 with the Reference Integration System 3105. Alternatively, or in addition, the therapeutic and/or diagnostic device displacement inside the lumen can be measured via the X-ray System 3103. Such displacement data are connected with the Reference Integration System 3105 from the Angiogram Data Storage 3108 via interface 3115. The Reference Integration System 3105 is connected to the Angiogram Data Storage 3108, Data Storage 3106, Diagnostic and/or Therapeutic System Data 3130 and an IT Infrastructure 3140 via, respectively, interfaces 3115, 3120, 3125 and 3145, respectively. A display 3107 can be connected to both the Reference Integration System 3105 and the X-ray System 3103 via, respectively, interfaces 3150 and 3151 for data output visualization and to provide for an operator/physician interface.

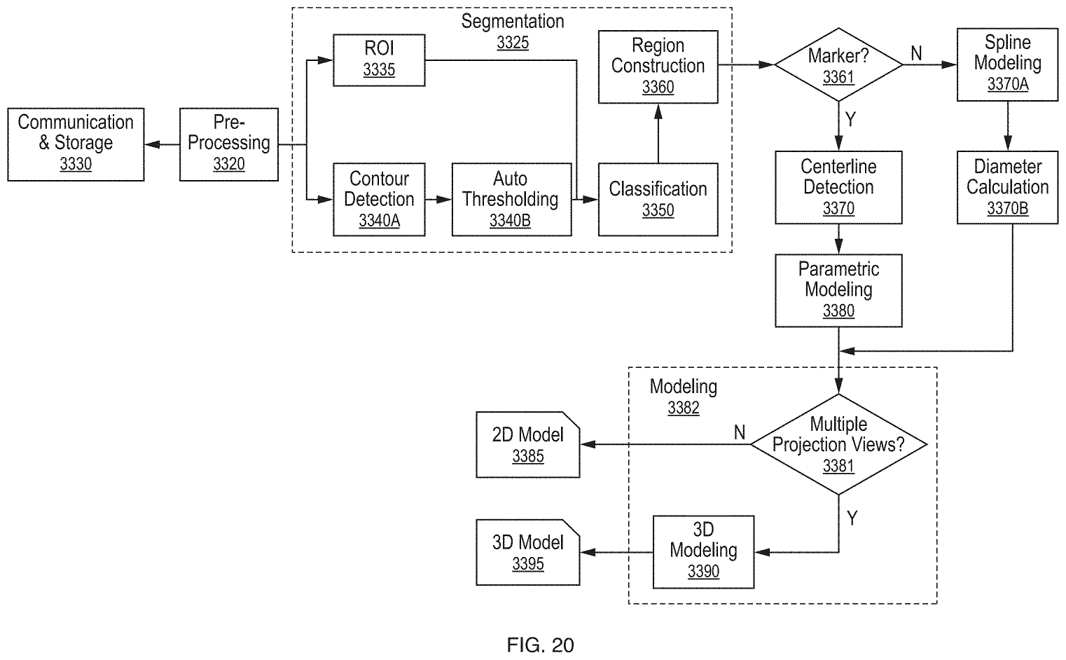

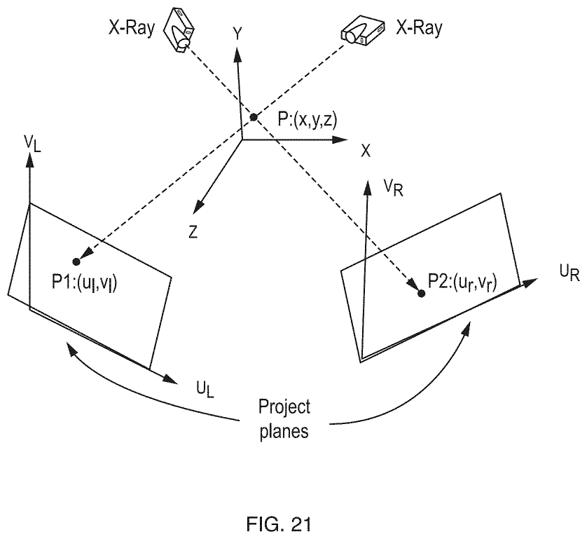

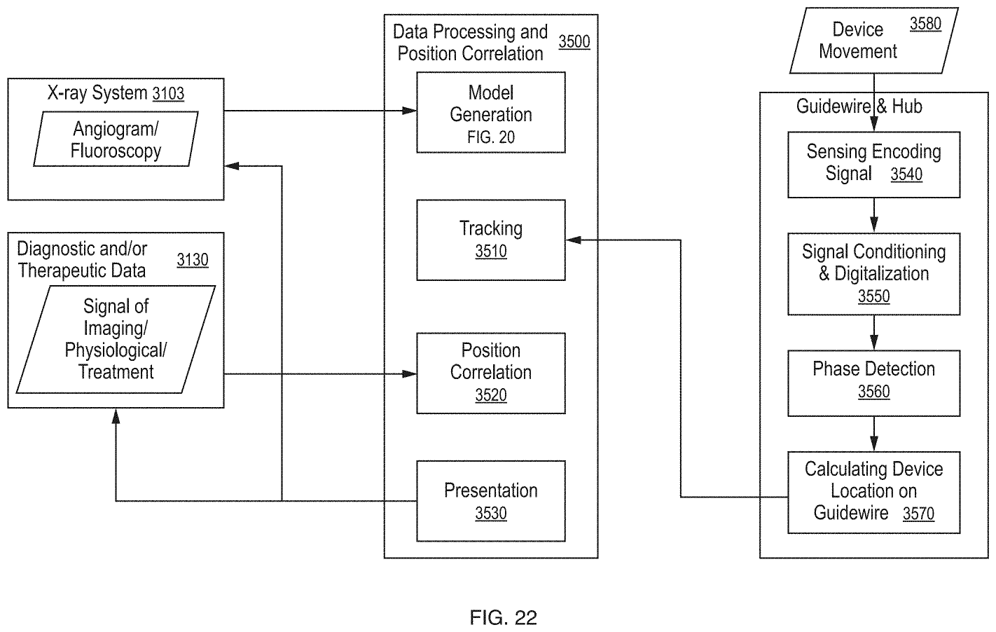

Based upon at least one angiographic image under at least one projected angle of the guidewire, guidewire-based modeling can be performed, as further described with respect to FIG. 20.

Communication and Storage

After the guidewire is deployed at a desired location inside the body lumen, the Reference Integration System 3105 can receive by electronic or wireless communication one or more angiogram/fluoroscopy images from the Angiogram Data Storage 3108, such as by DICOM-RTV (real-time DICOM) via interface 3115, and store the images (3330).

Pre-Processing

Pre-processing of the angiogram/fluoroscopy image(s) can be performed (3320) to remove noise from the images while preserving edges. In this step, the input images can be filtered, for example, with a 2D kernel or with an anisotropic filter. The output image(s) after pre-processing are then feed into a segmentation stage (3325).

Detection of Regions of Interest (ROI)

Features of interest (e.g., radiopaque markers, vessel lumen boundaries) can be separated from the rest of image and connected regions representing the features can be formed (3335). Filters can be used in this step (which can include, for example, Top-hat, Canny filter, Gabor filter, Phase congruency-based filter) to enhance and detect edges, and then a region detection algorithm can be used to separate regions of interest from background.

Contour Detection

An outline of guidewire can be detected (3340A), for example, using an active snake algorithm.

Auto-Thresholding

An automatic thresholding can be performed (3340B) to classify image components into one of three classes: radiopaque marker, lumen boundary, and background. The threshold value can be calculated, for example, with 2D multilevel Otsu's method.

Classification

Images from the region of interest (3335) and auto thresholding (3340B) steps can be combined for classification (3350). The classification can separate objects of interest (e.g., marker, lumen border) from background. Region algorithms can be used to link classified pixels belonging to an object of interest into connected regions. Constraints (such as known dimensions and/or spacing of radiopaque markers) can be applied to improve the degree of linkage.

Region Construction

Morphological operations, such as erosion and dilation, can be performed (3360) to form regions representing guidewire and lumen border. Background can be removed.

Centerline Detection and Modeling

The center line of the vessel lumen and/or each segment of the markers can be detected (3370). For example, a Hessian matrix algorithm can be used to detect the line segments. Depending upon an output at marker decision (3361), centerline models can be established via either parametric modeling (3380) for the marker(s) or via spline modeling (3370A) for the lumen wall. Spline fitting (3370A) can be used to approximate the two border lines of the vessel lumen, followed by calculation of vessel lumen widths (diameters) (3370B) at pre-measured locations along the vessel. The width values can be stored. When multiple views are available, widths can be calculated and stored for each view. Parametric modeling (3380) can include use of a Hough transform to determine parametric equations for the center line of each of the marker segments. As a result, 2D model(s) for radiopaque markers and/or vessel lumen can be established (3385) with calculated shape and dimension. Modelling (3382) can provide for a 2D model, a 3D model, or both. Optionally, when images from two or more known projections are available (3381), the above process (3320-3380) can be applied to each image to produce a 2D model for marker segments and/or lumen, which can then be processed (3390) to construct a 3D model (3395). The process to reconstruct a 3D model from 2D images (3390) can be performed as further described herein, such as with reconstruction by direct linear transform (DLT).

As shown in FIG. 21, a point P can be projected into 2 image planes U_{L}V_{L} and U_{R}V_{R} at points P1 and P2 An intersection of the projecting lines (\vec{P1P},\vec{P2P}), which is the location of P, can be found.

A Direct Linear Transform (DLT) can be used to solve the above problem. The projected coordinate can be written by the following equalizations:

$$\begin{matrix}u = \frac{{L}_{1} x + {L}_{2} y + {L}_{3} z + {L}_{4}}{{L}_{9} x + {L}_{10} y + {L}_{11} z + 1} & ( 1 ) \\ v = \frac{{L}_{5} x + {L}_{6} y + {L}_{7} z + {L}_{8}}{{L}_{9} x + {L}_{10} y + {L}_{11} z + 1} & ( 2 )\end{matrix}$$

The symbols L₁ to L₁₁ are DLT parameters. Because (u, v) is known, the object coordinate (x, y, z) can be calculated once the parameters (L₁, L₁₁) are measured. In linear mathematics, at least 6 points are needed to solve (L₁, L₁₁). In one representative embodiment, assuming N points are acquired, a matrix equation for L can be assembled using the following formula:

$$\begin{matrix}Point 1 \{ \\ Point 2 \{ \\ Point N \{\end{matrix} \underbrace{[ \begin{matrix}{x}_{1} & {y}_{1} & {z}_{1} & 1 & 0 & 0 & 0 & 0 & - {u}_{L 1} {x}_{1} & - {u}_{L 1} {y}_{1} & - {u}_{L 1} {z}_{1} \\ 0 & 0 & 0 & 0 & {x}_{1} & {y}_{1} & {z}_{1} & 1 & - {v}_{L 1} {x}_{1} & - {v}_{L 1} {y}_{1} & - {v}_{L 2} {z}_{1} \\ {x}_{2} & {y}_{2} & {z}_{2} & 1 & 0 & 0 & 0 & 0 & - {u}_{L 2} {x}_{2} & - {u}_{L 2} {y}_{2} & - {u}_{L 2} {z}_{2} \\ 0 & 0 & 0 & 0 & {x}_{2} & {y}_{2} & {z}_{2} & 1 & - {v}_{L 2} {x}_{2} & - {v}_{L 2} {y}_{2} & - {v}_{L 2} {z}_{2} \\ & & & & & ⋮ & & & & & \\ {x}_{N} & {y}_{N} & {z}_{N} & 1 & 0 & 0 & 0 & 0 & - {u}_{LN} {x}_{N} & - {u}_{LN} {y}_{N} & - {u}_{LN} {z}_{N} \\ 0 & 0 & 0 & 0 & {x}_{N} & {x}_{N} & {x}_{N} & 1 & - {v}_{LN} {x}_{N} & - {v}_{LN} {y}_{N} & - {u}_{LN} {z}_{N}\end{matrix} ]}_{2 N \times 11} \underbrace{[ \begin{matrix}{L}_{1} \\ {L}_{2} \\ {L}_{3} \\ {L}_{4} \\ {L}_{5} \\ {L}_{6} \\ {L}_{7} \\ {L}_{8} \\ {L}_{9} \\ {L}_{10} \\ {L}_{11}\end{matrix} ]}_{11 \times 1} = \underbrace{[ \begin{matrix}{u}_{L 1} \\ {v}_{L 1} \\ {u}_{L 2} \\ {v}_{L 2} \\ ⋮ \\ {u}_{LN} \\ {v}_{LN}\end{matrix} ]}_{2 N \times 1} . \qquad ( 3 )$$

Equation (3) is denoted as:

F_{(2N,11)}L_{(11,1)}=g_{(2N,1)} (4)

Similar matrix equations can be written for R(ight) projection (P2).

Equation (4) can be solved to find solution of L(eft) (and similarly R) by pseudo inverse method (or SVD decomposition), e.g.:

L=(F^{T}F)⁻¹F^{T}g (5)

With L and R solved, coordinates of point in object space can be calculated from:

$$[ \begin{matrix}{L}_{1} - {L}_{9} {u}_{L} & {L}_{2} - {L}_{10} {u}_{L} & {L}_{3} - {L}_{11} {u}_{L} \\ {L}_{5} - {L}_{9} {v}_{L} & {L}_{6} - {L}_{10} {v}_{L} & {L}_{7} - {L}_{11} {v}_{L} \\ {R}_{1} - {R}_{9} {u}_{R} & {R}_{2} - {R}_{10} {u}_{R} & {R}_{3} - {R}_{11} {u}_{R} \\ {R}_{5} - {R}_{9} {v}_{R} & {R}_{6} - {R}_{10} {v}_{R} & {R}_{7} - {R}_{11} {v}_{R}\end{matrix} ] [ \begin{matrix}x \\ y \\ z\end{matrix} ] = [ \begin{matrix}{u}_{L} - {L}_{4} \\ {v}_{L} - {L}_{8} \\ {u}_{R} - {R}_{4} \\ {v}_{R} - {R}_{8}\end{matrix} ] . \qquad ( 6 )$$

The left matrix in equation (6) is not square and can be decomposed or pseudo-inversed to solve (x,y,z) in object coordinate space.

At least 2 points can be chosen from each segment (e.g., a radiopaque marker as a segment). The points from each segment can then be linked to form a line section model for the segment, and spaces between any two markers can be estimated using polynomial fitting.

Optionally, a machine learning model can be trained and be inferenced for segmentation. A machine learning model can replace steps 3320 to 3360. For example, the machine learning algorithm U-net, an effective segmentation model for medical images, can be used.

Image blurriness resulting from measurement error for the location of P can be reduced when using a 3D modeling method described herein. The locations U_{L}, V_{L}, U_{R}, and V_{R} each comprise a degree of error in the location measurement, σU_{L}, σV_{L}, σU_{R}, and σV_{R}, respectively. By comparing the error of each location measurement at a different angle, standard error measurement reduction methods known in the art can be applied to the locations to reduce the degree of error in measuring P, thereby yielding a clearer image.

For modeling the body lumen, widths can be used to generate a stripe model (2D). Widths corresponding to the same locations can be measured (or interpolated) and can then be used to generate a tubular model (3D) of the vessel.

Labeling guidewire markers for ML (machine learning) training can optionally be performed. An ML training on guidewire markers can include a process in which the guidewire markers are processed through an algorithm that adjusts relative positions of the guidewires based on translocation data. The marked guidewire markers can be used to construct a model of the guidewire within the patient. The ML model can be used to generate a guidewire model.

When a device is travelling parallel to the guidewire, its location on the guidewire can be tracked and presented to users in real-time. If diagnostic devices are used, their modality data (which can include, for example, an image or a waveform) can be co-registered according to the therapeutic and/or diagnostic device location, and multi-modality data from the same location can be presented. A method for Data Processing and Position Correlation 3500 is illustrated in FIG. 22.

Device Movement (3580) and Tracking (3510)

As the therapeutic and/or diagnostic device travels parallel to the guidewire (3580), a signal modulated by linear displacement encoding markers (e.g., markers 2250, 2340, 2470) can be generated by the sensor (e.g., sensors 2290, 2308, 2480) and the signal communicated (3540). The signal can be sampled and modulated (3550), and then the conditioned and digitalized signal can be decoded (3560), after which the device displacement can be calculated relative to the encoding (3570). The device displacement calculation can be based on the linear encoding signals, and the device location calculation can be based on a known relationship of a linear distance (or dimensions) between the displacement encoding markers and any of the radiopaque markers. The calculation can further be based on the therapeutic and/or diagnostic device's 1-D linear coordinate on the guidewire and a timestamp of the distance (the time when its corresponding modulated signal was received). The calculation can be performed before data is sent to the Reference Integration System 3105 (FIG. 18). The Reference Integration System 3105 can calculate the 2D/3D coordinates of the therapeutic and/or diagnostic device using the 1-D linear coordinate and the 2D/3D model of the guidewire. Note that the disclosed linear encoding system and method described above is one example of how displacement and location can be determined. Device location information received from any source (e.g., a pull-back sensor, an encoding sensor, etc.) can be used, provided the location can be referenced to the radiopaque markers on the guidewire and can be received by the Reference Integration System 3105 to be tracked on the 2D/3D model of the guidewire.

Position Correlation (3520)

One or a plurality of images (or physiology signals, or treatment device signals) can be acquired from other systems, as shown with regard to Diagnostic and/or Therapeutic System Data 3130 in FIG. 18. The images (or physiology signals, or treatment device signals) can be time stamped and correlated with the location data acquired at the closest time instances. Optionally, system delays can be considered to improve accuracy. The diagnostic/therapeutic data can be further correlated to corresponding locations on the radiopaque marker 2D/3D model.

Presentation (3530)

As illustrated in FIG. 22, one or a plurality of simulated device images can be superimposed in real-time onto the guidewire model for display (3530) (e.g., as shown in the example displays of FIGS. 2B, 14, 15, 16). An operator can elect to view real-time or about real-time diagnostic information at any location on the guidewire marker model. Optionally, the operator can manipulate viewing parameters (e.g. enlarge, reduce, rotate) in real-time or about real-time. FIG. 22 depicts an overall data processing method for converting one or a plurality of simulated device images onto the guidewire marker model.

Device position tracking in the form of a visual real-time or about real-time illustration with distance measurements can be integrated with the angiogram vasculature imaging, as illustrated in the example composite angiogram image shown in FIG. 19. While a device, such as a stent on a deflated delivering balloon, is travelling parallel to a guidewire 3220 with markers 3230, travel distance data can be transmitted from the guidewire 3220 to the Reference Integration System 3105. Upon receipt of the travel data, distance measurements can be calculated and displayed in real-time, as described above. An illustration 3240 of the therapeutic and/or diagnostic device (or an illustration representing its location) can be simultaneously superimposed on to the composite angiogram image 3250 according to the therapeutic and/or diagnostic device real-time position. The distance, along with other calculated data, can be displayed (e.g., display 107, 3107). The therapeutic and/or diagnostic device illustration 3240 superimposed onto Angiographic images with precise position co-location can serve as a visual representation and navigation during inactive fluoroscopy. The therapeutic and/or diagnostic device illustration position and the associated measurements can be continuously or periodically updated by the Reference Integration System 3105 throughout a procedure.

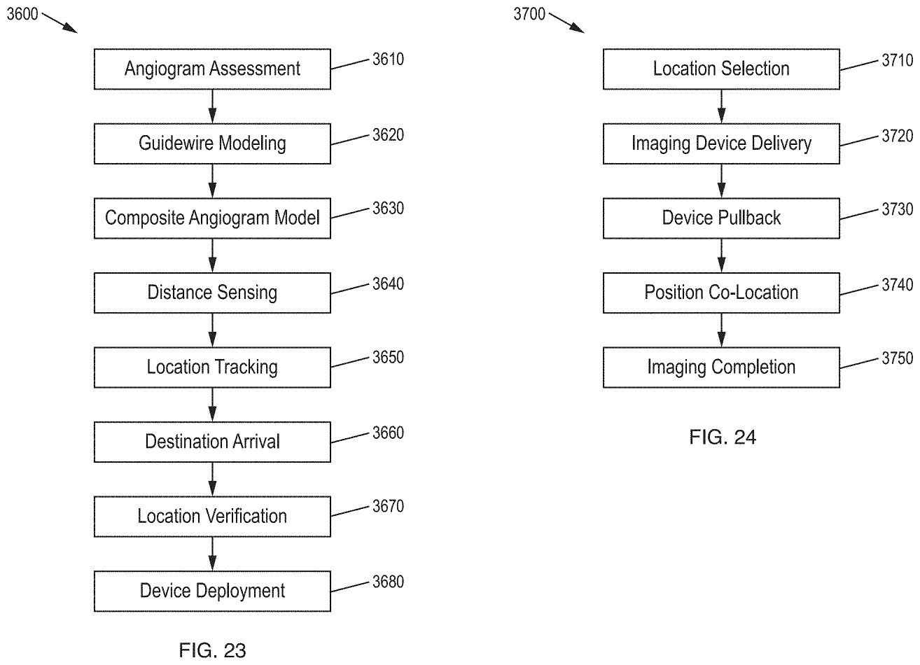

Using complex percutaneous cardiology interventional (PCI) imaging as an example, a workflow for a guided procedure 3600, including associated multiple diagnostic and/or therapeutic device position co-location and display, is shown in FIG. 23. X-ray external body imaging is typically used in cardiology intervention workflows in catheterization laboratory environments. An example of an X-ray interventional image guided system is the Innova™ IGS (GE Healthcare). After identifying an intra-vascular area of interest via a standard X-ray imaging assessment process with a conventional X-ray interventional image guided system, a guidewire with plurality of radiopaque markers (and, optionally, an embedded encoding sensor at a distal end) can be advanced and situated at the area of interest under live X-ray imaging (3610), as per standard PCI workflow. Fluoroscopy imaging can be activated at the desired imaging orientations (projections) while a contrast agent moves through the vasculature being examined. Both the guidewire, along with the plurality of the radiopaque markers, and the borders of lumen tissues with similar radiodensity can be delineated. The fluoroscopy images that contain both the lumen and the guidewire with a plurality of radiopaque markers at the to-be-treated area of the vasculature can be captured and recorded with one or multiple different projection angles. The imaging information obtained in step 3610 can be transferred to the Reference Integration System 3105 for processing and guidewire modeling (3620). In particular, 2D and/or 3D guidewire modeling via the plurality of radiopaque markers can be carried out through the process steps shown in FIGS. 20 and 21. The corresponding 2D and/or 3D vessel segment model can also be established. The guidewire modeling data can be superimposed with the corresponding X-ray image to form a composite image of choice (e.g., as shown in FIG. 19) (3630). With a known dimension and spacing of the plurality of the markers, a linear distance scale along the vessel relative to a reference point per the user's choice can be established on the composite image and can be displayed on, for example a boom display (e.g., display 3107).

For a therapeutic device delivery phase of the process 3600, for example, a balloon dilatation catheter can be delivered. An example of a balloon dilation catheter is the Coyote™ balloon dilation catheter (Boston Scientific). With the composite image remaining displayed, the X-ray system can be switched to inactive. The balloon catheter is advanced from a proximal end of the guidewire to a distal end of the guidewire and position sensing of the balloon catheter can be activated when, for example, an optical marker inside the balloon catheter shaft is detected by the optical sensor on the guidewire (3640). As the balloon travels along the guidewire, its optical markers travel past the optical sensor on the guidewire and a position of the balloon relative to the catheter can be detected via optical signal emission and reception by the guidewire sensor. The received signal can be transmitted to a signal processing component at a proximal end of the guidewire (e.g., Hub 3109) via optical fibers running through an inside the guidewire. The optical signal can be converted to an electrical signal at the Hub 3109. The data can then be transmitted from the Hub 3109 to the Reference Integration System 3105 (e.g., via Bluetooth or a wired connection).

Based upon the known dimension of the balloon and known embedded optical marker sequence of the balloon catheter, the balloon travel distance can be decoded from the electrical signal to obtain a linear displacement value. Because of the guidewire radiopaque marker coordinate system that is pre-established on the composite image, as well as the balloon displacement measurement with known starting and finishing points, a location of the balloon catheter can be calculated and identified on the composite image real time and/or near real time (3650). A representation or illustration of the balloon catheter (e.g., illustration 3240) can also be generated with a scaled real dimension on the 2D/3D guidewire model and corresponding to the balloon catheter location. The balloon catheter illustration 3240 can be superimposed and displayed on the composite image to represent the real-time or about real-time balloon location while the X-ray is inactive. The balloon displacement reading, a distance from the balloon to the target vessel location per a user's selection, and an illustration representing device movement can be updated while the X-ray stay inactive. The Reference Integration System 3105 can optionally signal the operator/physician when the balloon reaches the target location (3660). After arriving at the targeted treatment site, a location verification (3670) can optionally be performed prior to balloon deployment with live X-ray image capture. The live X-ray image data can be received by Reference Integration System 3105. The balloon live location information can be integrated with the pre-established guidewire and lumen 2D and/or 3D model. The balloon illustration location and the associated position information can be adjusted real-time or about real-time, if needed. An updated balloon illustration, alone with other updated location information, can be superimposed on an X-ray image per user's choice and displayed. Optionally, a location verification (3670) can be performed more than once, or anytime, per a user's preference, during balloon advancement on the guidewire. The balloon deployment is carried out (3680) after balloon arrival and the targeted treatment site and, optionally, after location verification. The composite imaging data, including balloon location and illustration, can be processed and updated in real-time or about real-time, recorded, and stored throughout the workflow 3600 by Reference Integration System 3105.

In a situation in which the X-ray system images are at a projection that varies from the initial angle when the location verification (3670) is performed. Reference Integration System 3105 can follow the same procedure descripted above to update the 2D guidewire model based upon the new X-ray image and projection. If the previous model is 3D, the model can generate and display the 2D model at the desired projection. An associated device position and distance information under the new projection can also be updated and displayed accordingly. Illustrations of the endoluminal device (e.g., balloon catheter, diagnostic device) can be adjusted accordingly as the device moves to a desired location while keeping X-ray inactive.

Intravascular imaging and/or physiology assessments are often carried out as part of a diagnostic procedure for further lumen assessment and treatment strategy determination. A workflow for a guided diagnostic procedure 3700 is shown in FIG. 24. The guided procedure 3700 can be performed before treatment delivery (e.g., prior to balloon delivery, prior to step 3640) and/or after treatment delivery (e.g., following balloon delivery, following step 3680). The workflow 3700 of FIG. 24 is described with respect to an example implementation with an endoluminal diagnostic IVUS imaging probe being delivered to verify treatment. An example of an IVUS catheter for use in such a procedure is the Eagle Eye Platinum paired with the Core Mobile stand-alone system (Philips Healthcare).

For example, after completion of a balloon dilation and retrieving process, as described with respect to workflow 3600, an operator/physician can select a desired lumen location from the established composite angiogram image for an IVUS imaging sensor to target (3710). In this example, the balloon dilation location from the previous workflow is the IVUS imaging target. With embedded optical markers included in a shaft of the IVUS catheter, catheter displacement relative to the guidewire sensor can be detected. Under the same distance sensing, location tracking, destination arrival, and location verification processes described in 3640, 3650, 3660 and 3670 of FIG. 23, the IVUS imaging sensor is placed at the desired lumen location (3720) and a starting point is established for catheter pullback. With X-ray inactive, the IVUS imaging sensor is pulled back according to standard IVUS imaging procedures (3730). For example, the pullback can be performed manually by an operator/physician or automatically by an automatic pullback device. Pullback movement of the IVUS catheter can be detected inside the lumen by an optical sensor of the guidewire generated in response to the IVUS optical markers. The optical can be converted to an electrical signal and transmitted to the Reference Integration System 3105 via the Hub 3109. The IVUS imaging sensor position tracking can be calculated via displacement data integration with the pre-established 2D/3D model of the guidewire (3740). The imaging sensor location and the associated position information can be superimposed and displayed on the composite angiogram image in real-time or near real-time while the X-ray is inactive (3750).

FIG. 25 illustrates an example of co-location and display 3800 (illustrating an output of steps 3740, 3750) among different systems and/or devices concurrently and while X-ray is inactive. With a known dimension and spacing of the plurality of the markers on the guidewire, a linear distance scale along the vessel relative to a reference point per the user's choice can be established on a composite X-ray angiogram image 3810 and on a 3D guidewire and lumen model 3820 with the plurality of the radiopaque markers 3830 shown on the 3D model. Continuing with the example implementation involving an IVUS catheter, an initial position 3801 of the IVUS imaging sensor, before pullback, can be obtained from an imaging sensor location verification step. Based upon the displacement and the associated endpoint 3805, the imaging sensor location tracking can be established. As the imaging sensor is generating both an IVUS cross-sectional view 3840 and a longitudinal view 3850 during pullback, a guidewire marker trajectory, along with an imaging sensor illustration, is overlaid with the IVUS longitudinal view based upon the established coordinate system. The imaging sensor location and the associated linear distance information can be displayed on IVUS longitudinal view 3850 while X-ray is inactive. Furthermore, the balloon dilation location segment 3860 from the previous step can be precisely co-located and overlaid on the IVUS longitudinal view. The X-ray composite image, guidewire model and the IVUS longitudinal view can be displayed from any X-ray projection angle and/or IVUS longitudinal viewing angle per the user's choice. The corresponding device location, lumen location information, and/or position information obtained from the previously-described example workflow can be precisely co-located and displayed in real-time or near real-time, or concurrently as the imaging sensor is pulling back. Unlike the current pullback distance measured from a proximal end of the imaging catheter or via live X-ray, the imaging sensor movement detected by the guidewire optical sensor at a distal end of the devices inside the lumen represents a precise device location and displacement, which can eliminate measurement inaccuracies that result from the current pullback method. Furthermore, the precise displacement measurement provided by the guidewire is live X-ray independent and offers flexible and customizable IVUS imaging workflows to the operator/physician that are not achievable by the current procedure. With X-ray continuing to be inactive, a user can complete the IVUS imaging process with recorded imaging and co-location information. Users can apply the same workflow on other endo-luminal diagnostic devices such as OCT and/or FFR/iFR of their choice.

In a further example, a stenting procedure and associated post-IVUS imaging evaluation can be performed, as described in an example of the workflow 3900 depicted in FIG. 26. An example of a stent for use in such a procedure is the Synergy™ stent (Boston Scientific). A target vessel location can be identified by an operator/physician on the composite X-ray image 3810 and/or IVUS longitudinal view 3850, as shown in FIG. 25. With the X-ray system inactive, an operator/physician can deliver a stent balloon catheter to the desired a lumen location (3910) following a similar workflow as described with respect to FIG. 23. As the stent balloon catheter location is detected by the position sensor on the guidewire with X-ray system inactive, its position and associated linear displacement measurements can be co-located and displayed in real-time or near real-time (3920), optionally along with a stent location illustration, on a composite X-ray image and composite IVUS longitudinal view, as previously obtained (FIGS. 24 and 25). The Reference Integration System 3105 can update a position of the stent via the 2D/3D guidewire model during to stent location verification (3930). Corresponding data processing, including data receiving, model computation, co-location integration, and display can be performed (3940). During and/or after stent deployment (3950), an X-ray image can be taken to evaluate the stent deployment, followed by stent apposition and vessel evaluation by IVUS imaging (3960). Since vessel positions as indicated within the X-ray image(s), IVUS image(s), and device position(s) can be precisely co-located via the 2D/3D guidewire model, cross-evaluation among the several modalities can be performed prior to, during, and/or following any diagnostic and/or therapeutic procedure.

As an example, a stent segment 3870 can be deployed and a position 3875 can be co-located with lumen locations of an IVUS image and a balloon dilation segment 3860, as shown in FIG. 25. This integrated format can provide for ease of use and introduce novel clinical insights not previously available. Furthermore, this complex percutaneous intervention procedure described can offer increased flexibility and customizable workflows with minimum radiation exposure to users, patients, and operating environments.

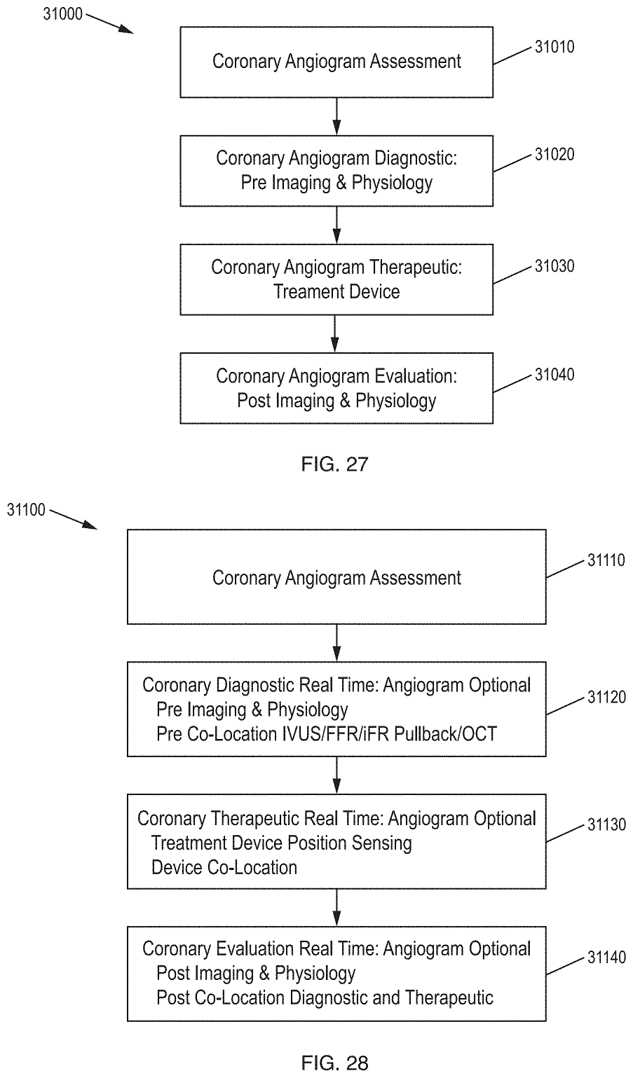

FIG. 27 and FIG. 28 depict a comparison summary of a workflow of a standard PCI procedure 31000 (FIG. 27) and a PCI procedure 31100 with the benefit of a congruent location system, as described above (FIG. 28).

FIG. 27 shows a complex percutaneous interventional cardiology procedure as a representative standard, current method 31000. After finalizing an area of interest, a guidewire is advanced and situated at the area of interest by means of a standard angiogram assessment (31010). With the live X-ray imaging visualization, intra-coronary diagnostics, such as IVUS imaging, OCT, and/or FFR are performed (31020). By reviewing X-ray images and the diagnostic data displayed independently on each modality system, an operator/physician mentally integrates the data to determine a treatment strategy. Based upon a highly operator-dependent treatment decision, treatment devices (e.g., a balloon or stent) are delivered to about the target location under the guidance of live X-ray (31030). Post-treatment evaluation is performed (31040) to assess the clinical effectiveness and potential risk. In particular, the imaging and/or physiology device is again delivered to the treated locations under live X-ray guidance. The operator/physician integrates the therapeutic data with the post-stenting evaluation data mentally for each estimated lumen location of interest.

FIG. 28 shows the workflow of one representative method 31100 using the provided devices and systems, where the advantages over the method described in FIG. 27 are also described. The real-time or about real-time device co-location sensing system and method provided enables each step of the complex percutaneous intervention procedure to be highly integrated, with minimum dependency on X-ray Angiogram and fluoroscopy, thereby reducing X-ray exposure to the operator/physician and/or patient. After an initial assessment (31110) under X-ray Angiogram, live X-ray navigation becomes optional, and diagnostic procedures (31120), therapeutic device delivery and deployment (31130), and post-treatment evaluation (31140) can be performed without live X-ray. Accordingly, radiation exposure can be greatly reduced. Furthermore, a precise real-time sensor location (and associated measurements/imaging for a given location) can be determined and provided to the user. Further still, precise co-location among multiple systems (e.g., X-ray images, diagnostic imaging, physiology assessments, and therapeutic device deployments) can be provided. Thus, a full suite of precise and correlated comprehensive clinical information can be provided to physicians to optimize treatment strategies, treatment deployments, and clinical evaluations in real-time throughout PCI procedures. Real-time or about real-time co-location for decision making, on-target delivery and deployment, and minimum X-ray radiation exposure are some of the advantages of the provided methods over the current, standard PCI method described in FIG. 27. Real-time or about real-time co-location also provides for a solution to previously unmet operator/physician needs for endo-luminal intervention procedures.

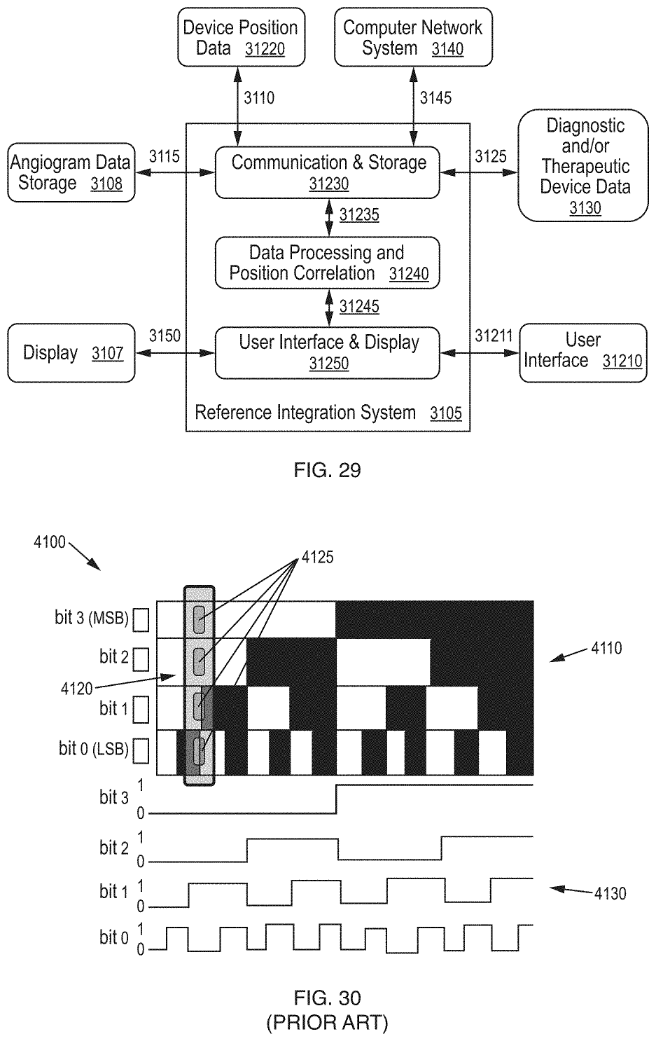

The systems described herein can provide for data acquisition, modeling, procedure guidance, precision lumen location correlation, and position information display with minimum radiation. The Reference Integration System 3105 can include several subsystems, as illustrated in FIG. 29: (1) Communication and Storage subsystem (31230); (2) Data Processing and Position Correlation subsystem (31240); and (3) User Interface and Display (31250).

The Communication and Storage subsystem (31230) can interface with external data streams 3110, 3145, 3115, 3125, store raw data on system memory banks, and provide an internal data stream 31235, allowing the Data Processing and Position Correlation subsystem (31240) to access different streams of data, save the processed data in the system memory bank, and interface with external storage as needed.

The Device Position Data interface (3110) can interface with a guidewire, therapeutic device and/or diagnostic device to obtain position information input (31220) and store corresponding data in the system memory bank for processing by a Data Processing and Position Correlation subsystem (31240).

The Computer Network System interface (3145) can transmit and/or receive data from local and/or external network storage systems containing information for signal processing in subsystems 31240 and 31250. The data can be real-time or about real-time, and can be acquired from different procedures and/or steps such as but not limited to ECG (Electrocardiogram), Doppler, FFR (Fractional flow reserve), FFR-CT (Fractional flow reserve-computed tomography), IVUS (intravascular ultrasound), and OCT (Optical coherence tomography). The Computer Network System interface (3145) can provide for saving raw data and final processed data from memory banks to local and/or external storage systems for further data processing from other therapeutic and/or diagnostic systems.

The Angiogram Data Storage interface (3115) can interface with the Angiogram Data Storage (3108) to obtain real time and/or about real time Angiogram data and store the data in the system memory banks processing by the Data Processing and Position Correlation subsystem (31240).

The Diagnostic and/or Therapeutic System Data interface (3125) can access diagnostic and therapeutic information before, during, and after procedures from a Diagnostic and/or Therapeutic system (3130), such as but not limited to ECG (Electrocardiogram), Doppler, FFR (Fractional flow reserve), FFR-CT (Fractional flow reserve-computed tomography), IVUS (intravascular ultrasound), and OCT (Optical coherence tomography). The Diagnostic and/or Therapeutic System Data interface (3125) can also accesses the therapeutic and/or diagnostic device Position Data or portion thereof that is unique to the configuration of the diagnostic and therapeutic systems and devices, such as but not limited to catheter pullback distance (i.e., as obtained at the proximal end of the catheter via an apparatus as part of the diagnostic/therapeutic system).

The Data Processing/Position Correlation subsystem (31240) can serve several functions. From the angiographic information (3115), including images of the radiopaque markers, the subsystem can establish 2D and/or 3D models of the flexible elongate instrument inside the lumen with dimension information and relative position to the lumen. The subsystem (31240) can receive position and/or displacement information pertaining to the therapeutic and/or diagnostic device in real-time or about real-time from any of: the Device Position Data (31220), the Diagnostic and/or Therapeutic System Data (3130), and the Communication & Storage (31230) via the interface (31235). The subsystem (31240) can integrate the position data with 2D and/or 3D models of the flexible elongate instrument and generating real-time or about real-time device position illustrations, including superimposition of the illustration with the 2D and/or 3D models. The subsystem (31240) can also generate position correlation display data via real-time or about real-time data integration among the 2D/3D model, simulated device illustration(s), diagnostic and therapeutic system data, and Angiogram data. The subsystem (31240) can also provide for input and processing of operator/physician-selected viewing options, such as 2D/3D, a projection of interest, viewing angles with device signals at any location, and/or other execution requests via the User Interface and Display subsystem (31250).

The internal data interface (31235) can serve as an interface between the Communication and Storage subsystem (31230) and the Data Processing/Position Correlation subsystem (31240). Raw data, which can include data from the Device Position Data interface (3110), Computer Network System interface (3145), Angiogram Data Storage interface (3115), and Diagnostic and/or Therapeutic System Data interface (3125), can reside in local memory bank within the subsystem (31230).

The Data Processing/Position Correlation subsystem (31240) can access raw data through the interface (31235). Processed data from the subsystem (31240) can be stored in the memory banks in subsystem (31230) through interface 31235.

The User Interface and Display subsystem (31250) can place the processed data (31245) from the Data Processing/Position Correlation subsystem (31240) in a proper format (3150) for display by the Display system (3107), such as in a graphical representation based upon User Interface data inputs (31211) from User Interface devices (31210). Operator/physician interface inputs can be embedded into the display data (3150) to display system (3107) or can be embedded into the operator/physician interface data (31211) to the User Interface (31210) as a separated display and control.

The provided systems and methods can be applied to any interventional procedure for a body lumen. Optionally, a GUI can be rendered on the Reference Integration System 3105 with components or controls to allow an operator to interact with the Reference Integration System 3105 via command control for execution, including providing for interfacing a lumen position correlation display with third-party diagnostic and therapeutic systems. A form of the visualization display system (e.g., display 3107) can vary and can be or include, for example, a monitor, mobile device, wearable device, and AR/VR head mounted device. The inputs from an operator/physician at an operator/physician interface 31210 can be executed via an electronic device, such as a computer, a server with a monitor, a host workstation, a controller with a monitor, and a third-party system operator/physician interface. An I/O can include a keyboard, joystick, mouse, touch display, project device, microphone, any consumer and/or wearable electronics, such as mobile phone, AR headwear, pointing device, and audio feedback, for communicating with the Reference Integration System 3105 for procedure control, data rendering and visual display, data storage, and basic data process functions. Such a connection mechanism can provide ease of use workflow with adequate customization flexibility on real-time or about real-time lumen position correlation and associated data processing steps for users throughout a guided procedure. The interface connections 3110, 3145, 3125, 31211, 3150 and 3115 with the Reference Integration System 3105 as shown in FIG. 29 can be established via various connection mechanisms such as cables, cell networks (4G, 5G), local and or wide area network (LAN and WAN), Bluetooth network or wireless.

A ML method can further comprise curve-fitting techniques to develop a model of the catheter within the body lumen. The curve fitting may be done manually or may be fully- or semi-automated. For example, on one X-ray image, 3-16 boundary points can be selected along the guidewire as guidewire markers. After placement of the boundary points, a cubic spline interpolation technique may be used to fit a curve between each of the boundary points. The curve may satisfy the following equation:

Sₙ(x)=aₙx³+bₙx²+cₙx+dₙ (7)

By solving the system of n equations, where n is the number of boundary points selected (either manually or automatically), a cubic spline curve can be obtained for the length of the catheter (i.e., the distance from the proximal end to the distal end of the therapeutic and/or diagnostic device).

A “boundary point” can be selected from an edge of an image feature or a centerline of the image feature. An image edge can be ascertained by methods known in the art, including methods that detect where the image brightness changes sharply or has discontinuities.

Optionally, a diagnostic and/or therapeutic device can further include pre-measured modeling data, which can be transmitted to the calculation unit (e.g., Reference Integration System 3105). While diagnostic devices typical to PCI procedures are described in the examples above, the diagnostic device can be of another modality, such as a 3D MRI or CT. Pre-measured modeling data can include distance signal information, which is what would be expected for the body lumen of a patient who was previously imaged using 3D MRI (magnetic resonance imaging) and/or CT (computed tomography scanning) based on the MRI, CT, or X-ray angiogram data of the patient.

Optionally, a relative position of a first flexible elongate instrument and a second flexible elongate instrument can be measured from a plurality of sensors, wherein a first sensor is on one of the flexible elongate instruments (e.g., sensor 120) and a second sensor is outside the body of the patient (e.g., sensor 126) and connected to the other of the flexible elongate instruments. A sensor outside the body of the patient can be, for example, part of a robotic arm, or a motor-drive position unit. Two sensors can be useful for ultra-tortuous body lumen, such as in the brain, where the displacement measurements done at the distal and proximal ends can be very different. A relative co-location identification within the body lumen provides precise displacement relative to the plurality of imaging markers, and such data can be communicated to assist in guiding the robotic arm to advance one of the two flexible elongate instruments.

### 4. POSITION ENCODING AND SINGLE-ELEMENT DETECTORS

Flexible elongate instruments can include single-element sensors to detect encoding on other flexible elongate instrument to provide for position information during an endoluminal procedure. The encoding can be of a single code track configured to provide for absolute position detection. Such a configuration can advantageously provide for location detection of instruments used, for example, in a percutaneous intervention procedure by providing for a compact form suitable for use on or with endoluminal instruments.

Absolute position encoding typically uses sequences of code lines of different widths, which are unique for different positions. For example, for a common binary position code, four code characters are needed for a code sequence that represents a position: a digit separator character, a “0” character, a “1” character, and a position segment separator character. For constant speed motion, time duration can be used as a substitute for a digit separator. For a pseudorandom sequence binary position code, a position segment separator character may not be needed because the sequence change from each additional digit can represent a new position. In short, for absolution position encoding as performed with existing methods, at least three code characters are needed.

Current technology for a single code track, absolute-position, binary encoding commonly uses array-type sensors to detect a sequence of code mark widths. An array-type sensor includes many light sensitive elements, or pixels, that can capture images of the code lines in at least one direction and thereby determine the width of each code line.

In some situations, a single-sensing-element detector can be used when a speed of relative movement between a code track and a detector is constant because a code line width can be calculated based on a time duration of a given signal level. If movement between the code track and code detector is random, then time duration cannot be used to determine code line width.

Most vascular, endoluminal medical devices have small profiles such that the device can be positioned and move within blood vessels. An array type sensor and its associated wirings do not fit within or on these devices. Additionally, when these devices are used, their speed of movement in a body lumen is typically not constant and cannot be predicted. In interventional medical procedures, accurate determination of an endoluminal diagnostic or therapeutic device's location inside of a body lumen can be important. There is a need for an absolute position encoding system that can meet the needs of being both small in profile and providing for accurate coding information with random movement. A position encoder incorporated into these devices can be very small and can accommodate limited room for wiring access.

FIG. 30 illustrates a commonly-used, multi-channel absolute position encoding system 4100. A 4-track, 4-channel encoding strip 4110 provides for a 4-bit binary signal with 16 positions. A detector 4120 includes four sensing elements 4125, each sensing element generating a signal output from its respective code track, as shown in output 4130. In this example, white marking represents the code character 0, and black marking represents the code character 1. The 4-code character in the 4-bit binary sequence is generated simultaneously.

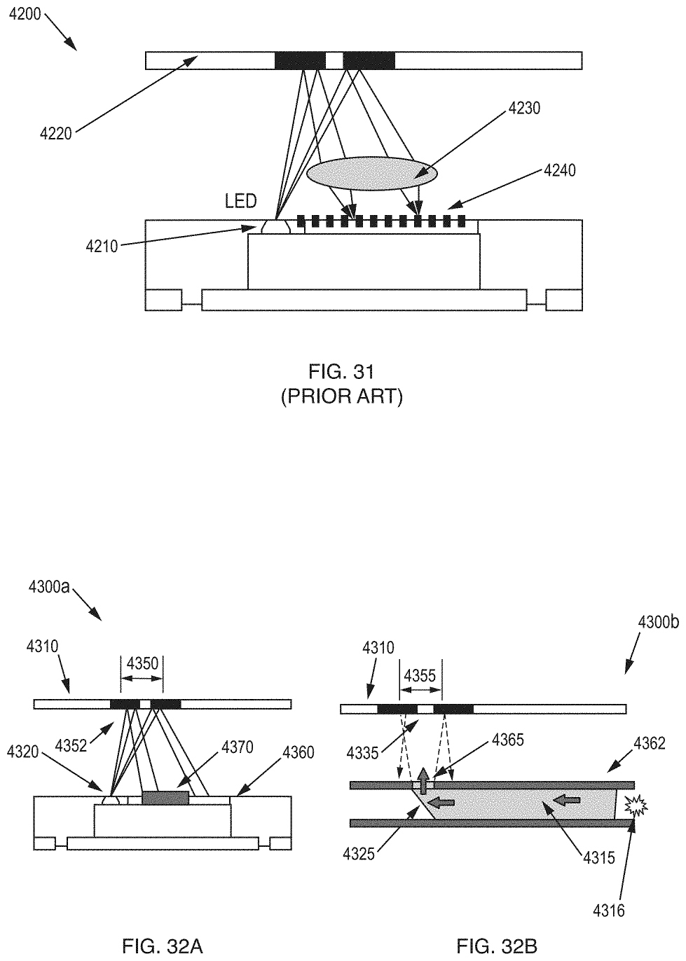

FIG. 31 illustrates a commonly used array-type sensor 4200 for absolute position encoding. A light source 4210 illuminates the code track 4220. Light from the light source 4210 is reflected off the code track, passes through an optical lens 4230, and is focused on an array-type sensor 4240. The array sensor 4240 includes several sensing elements, or pixels. Common array-type sensors can be constructed of CCD sensors, or CMOS sensors, for example. The array can also be either a linear array oriented in the direction of movement, or a two-dimensional array that can include thousands of pixels. Spacing information of the code lines are captured by the array sensor and conveyed to computer processor.

Examples of detectors with single-element sensors and encoding methods that can provide for absolute position determination are provided. The detectors can also allow for random speed movement. With a single sensing element sensor, a detector can be made small enough to be constructed into an endoluminal device. A determination of code line width that is not based on time information, but rather is based on reflected light intensity can be employed. Consequently, a determination of code line width can be made without being impacted by variations in in movement speed.

As used herein, the term “single-element sensor” or “single sensing element sensor” refers to a non-array sensor. A “single-element sensor” can be a single pixel sensor or a multiple pixel sensor that provides for a single output signal.

FIGS. 32A-B illustrate two examples of systems 4300a, 4300b with single sensing element sensors. As illustrated in FIG. 32A, a code track 4310 is illuminated by a light source 4320. Any type of light source can be used for illumination. The light is projected onto the code track 4310, illuminating a finite illuminated area 4352 that has a finite width 4350 in the direction of movement between the code track 4310 and the detector 4360. The reflected light from code track 4310 is captured by sensor 4370, which is a single-sensing-element sensor, also referred to as a single-pixel sensor. Optionally, multiple sensing elements or pixels can be used, but each sensing element or pixel does not provide a separate output; rather sensing is combined into a single output, or a single channel, such that the position information of each individual pixel is not captured.

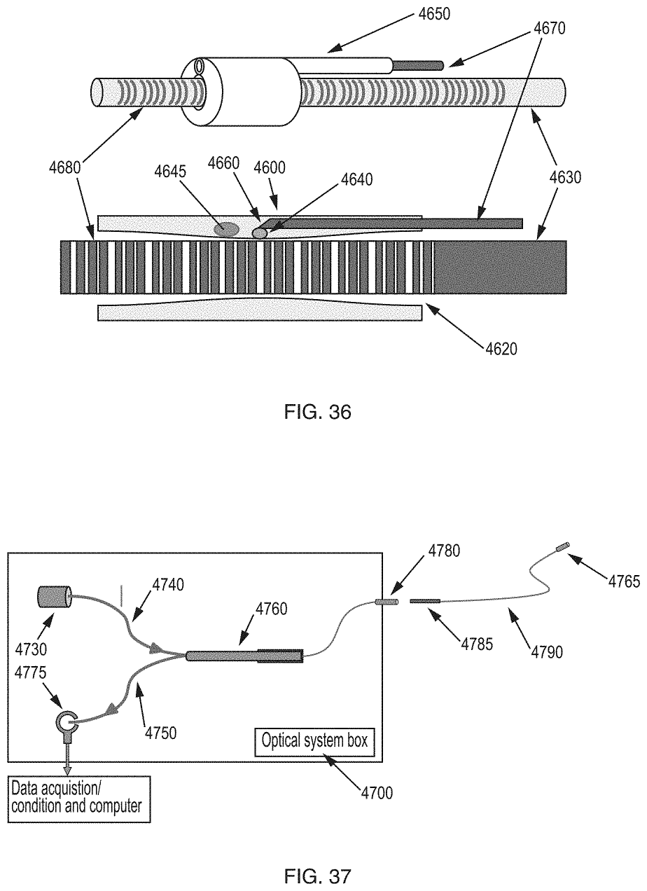

As illustrated in FIG. 32AB, a detector 4362 includes an optical fiber 4315 that transfers light from a light source 4316. A reflective surface 4325 is illustrated as a 45-degree polished end surface of the fiber 4315 having a reflective coating. The coating can be made from a number of materials, such as, for example, aluminum, silver, chrome, gold, platinum, etc., which can be applied to the surface via, for example, vacuum deposition. The light is reflected by the reflective surface 4325, exits a window 4365, and illuminates a finite illuminated area 4335 on the code track 4310. The finite area 4335 has a finite width 4355 in the direction of movement. A portion of the reflected light from code track 4310 re-enters the window 4365 and follows the optical fiber to a light sensor (see, e.g. FIG. 36). The optical fiber can transmit light from a light source to illuminate the code track and transmit the reflected light from the code track to a light sensor.

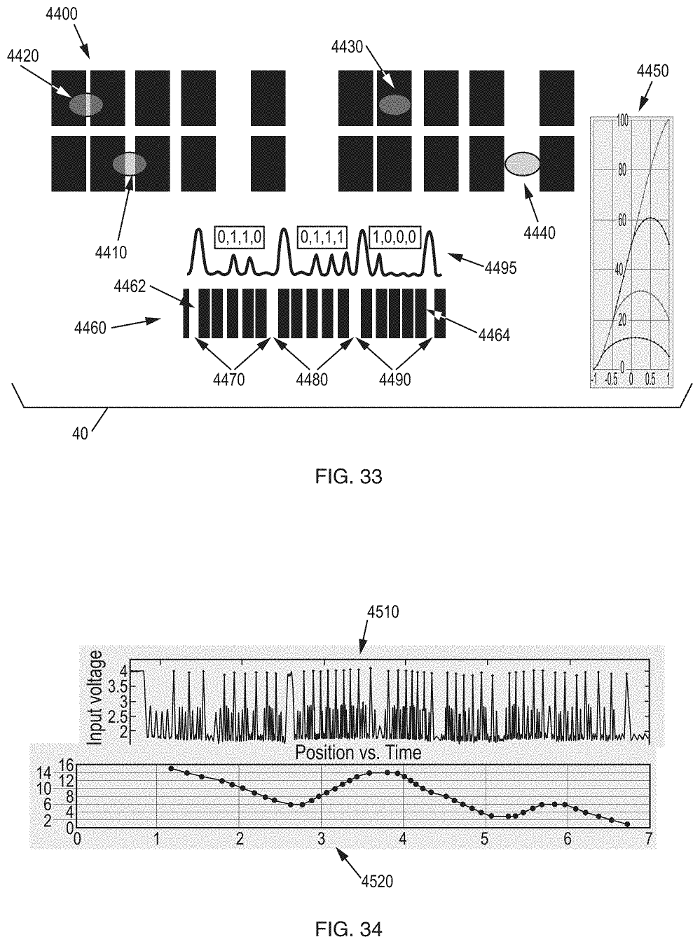

FIG. 33 is a schematic 40 illustrating a principal of operation using a single sensing element sensor to recognize different code characters based on the width of the code lines. A code track 4400 is illustrated with example light-sensitive areas 4410, 4420, 4430, 4440 as provided by a detector passing the code lines. A single sensing element can be sensitive to a finite area on the code track, adjacent to the sensor element. Such a finite area is referred to herein as a light sensitive area. Code markings outside of the light sensitive area are not detected by the sensing element. The light sensitive area can be produced by illumination of a finite area on the code track, or an area that is limited by a size of the sensor element, or a size of a mask or window placed in between the sensing element and the code track that defines an area on the code track for which the light can reach the sensing element.

When a marking width of either a high-reflectance surface or a low-reflectance surface is equal to or wider than the light sensitive area (4430, 4440), a fully-high signal and fully-low signal, respectively, are produced. A wider code line than that which produces a fully-high or fully-low signal does not change an output signal level by the sensor.

When a marking width does not fully cover a light sensitive area (4410, 4420), a partial signal is produced. When different marking widths are calibrated to produce different signal levels, the signal levels can be used to determine the marking width that produced the signal, and different marking widths can be used to represent different code characters. The example of the light sensitive areas shown by 4410, 4420, 4430, and 4440 are approximately circular, but it is understood that a shape of the light sensitive area can be modified depending on a light/sensor design and a use situation.

A graph 4450 displays a theoretical calculation of a light intensity profile change when a high-reflectance code line of full width, ½ full width, ¼ full width, and ⅛ full width passes a circular light sensitive area and when the code lines are flanked by full-width low reflectance code lines on either side.

A section 4460 of a 4-bit code track includes, for example, three position segments 4470, 4480, and 4490. In section 4460, the widest high reflectance code lines (e.g., code line 4462) represent the position segment separator code character. The low reflectance code lines (e.g., code line 4464) represent the digit separator code character. The narrowest, and intermediate high reflectance code lines represent binary code character “0” and “1” respectively. A reflected light intensity signal 4495 results when the detector passes the code track. The 4-bit position codes 0110, 0111, and 1000 represent three unique adjacent positions on the code track.

The example section 4460 provides for a binary position code that includes 4 signal levels for position encoding. For some encoding algorithms, such as a pseudo random sequence code, 3 signal levels can be sufficient.

FIG. 34 illustrates a decoded position vs. time result from a random movement between a 4-bit code track and an encoding detector having a single-element sensor. A reflected light intensity signal 4510 from the code track is shown adjacent to a position vs. time plot 4520 from the movement. As illustrated in this example, the random movement includes four direction changes, which can be determined based on comparison with prior neighboring code sequences.

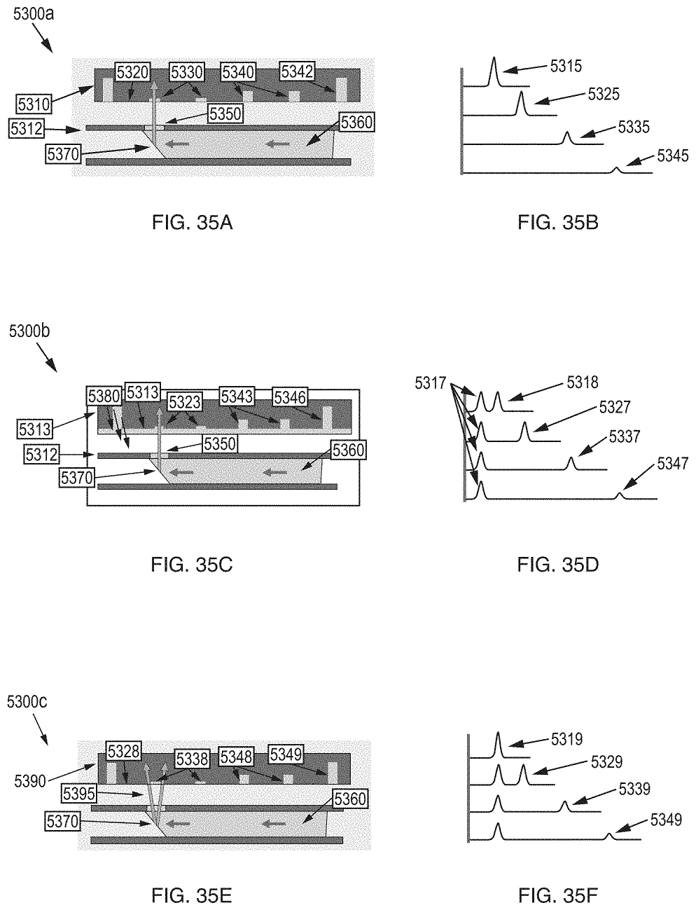

FIGS. 35A-F illustrate further examples of systems with single sensing element sensors that can generate a signal providing for absolute position information, including position, direction of motion, and speed of motion. Systems 5300a, 5300b, 5300c illustrate three different implementations of code track construction that can provide absolute position binary encoding for 4-bit sequences. The systems can make use of what is referred to herein as “OCT-based position encoding” in which code line engraving depths are detectable by an optical detector. A sensor can include a portion of a single optical fiber, through which light is transmitted from a source to the code track and through which reflected light is transmitted from the code track to a photo detector. While the examples described provide for encoding with 4-bit sequences, other types of binary encoding, such as pseudo-random sequences, can alternatively be provided by an OCT-based position encoding, as well as any number of bits of sequence length, which can be determined by a number of positions to be encoded.

FIG. 35B illustrates example reflected signals as detected by the system 5300a shown in FIG. 35A. The reflected signals are as detected from a single-pulse light emission from the sensor 5312 to a code track 5310 in which engraved code lines are wider than a beam width from the optical fiber. Light is transmitted by an optical fiber 5360 and reflected (as illustrated, for example, at 90 degrees by a 45-degree polished end surface 5370) through an optical window 5350 and towards the code track 5310. The code track has an outer surface 5320, shallow depth engraved code lines 5330, intermediate depth engraved code lines 5340, and deep engraved code lines 5342. When the sensor 5312 is maintained at a constant distance from the code track 5310 during relative movement, the reflected light signals from lines 5320, 5330, 5340, and 5342 are as shown by signals 5315, 5325, 5335, and 5345, respectively, in FIG. 35B.

As an example, a coding algorithm can be assigned such that signal 5315 represents a bit separator code character, 5325 represents a “0” code character, 5335 represents a “1” code character, and 5345 represents a position segment separator. When assigned as such, the code lines engraved in 5310 represent the binary sequence 0,0,1,1 in the illustrated example.

FIGS. 35C-D illustrate another example implementation. The code track 5313 in FIG. 35C differs from code track 5310 in FIG. 35A by having a translucent coating layer 5380 applied. The translucent layer can also reflect light from the sensor, producing a signal peak. Consequently, when light is reflected off the surface 5313, a shallow code line 5323, an intermediate depth code line 5343, and deep code line 5346, not only does each line produce its own peak (as shown in signals 5318, 5327, 5337, 5347 in FIG. 35D), but an additional peak 5317 from the translucent coating layer 5380 is produced. An advantage of this implementation is that the sensor 5312 need not to be maintained at a constant distance from code track 5313. The distance between the two signal peaks is not impacted by a distance between sensor 5312 and code track 5313 and can be uniquely different for each code line. Thus, a distance can also be used to represent different code characters.

FIGS. 35E-F illustrate another example implementation. The code track 5390 differs from the code track 5310 shown in FIG. 35A in that the engraved code lines are narrower than the light beam width. When the light beam 5395 is reflected off the code track surface 5328, a single peak in the reflected signal 5319 results. However, when the light is reflected off a code line, a portion of the beam is reflected off the adjacent surface 5328 and a portion of the beam is reflected off the code line, producing two peaks. A distance between the two peaks can be proportional to a depth of the engraved code lines. Signals 5329, 5339, and 5349 are reflected signals from code lines 5338, 5348, and 5349, respectively. In this example, the four different reflected signals can be used to represent four different code characters.

FIG. 36 illustrates an optical fiber based detector 4600 constructed into an interventional medical device system that includes a monorail catheter with a guidewire lumen 4620 and a medical guidewire 4630. Only the distal portion 4650 of the monorail catheter is shown here. A window 4640 at an interior surface of the guidewire lumen can allow light from a reflective end surface 4660 of the optical fiber 4670 to be reflected out and onto a coded surface 4680 of the guidewire 4630. When the monorail catheter is moving relative to the guidewire 4630, or vice versa, light carried by the optical fiber 4670 can be projected out of the window 4660 and reflected back through the window 4660 to the fiber 4670 to be carried back to a light meter. A light intensity modulation vs. time from the coded guidewire surface 4680 can be recorded and analyzed by a processor, which can then be used to calculate a location of the distal portion 4650 of the monorail catheter relative to the guide wire 4630.

Optionally, the detector 4600 can include an additional sensor 4645 configured to provide directional information of the guidewire 4630 (or other type of flexible elongate instrument). For example, the direction sensor 4645 can be a force gauge configured to provide an additive signal indicating advancement and/or a subtractive signal indicating retraction of the guidewire. The inclusion of a directional sensor in a system can provide for directional information with encoding that does not provide for directional information or can be used in conjunction with directional encoding. Directional sensors can be included in systems in which position encodings other than absolute position encodings are provided. As illustrated, the directional sensor 4645 is shown as being disposed at a distal portion of a flexible elongate instrument; however, a directional sensor can instead be disposed at a proximal portion of a flexible elongate instrument (e.g., at or near a pullback or push sensor, such as at or near sensor 126, FIG. 1).

FIG. 37 shows an example optical fiber based system and illustrates light passage through the system. An optical system box 4700 includes a light source 4730 that provides a beam of light for introduction into an optical fiber 4740. It is understood that the light source can be an LED source or a laser source, or any other type of light source with sufficient illumination power. Where OCT-based position encoding is used, the light source can be an OCT light source. Time domain OCT light sources typically provide monochromatic light. Frequency domain OCT light sources typically provide polychromatic light.

The optical fiber 4740 can connect to an optical fiber coupler 4760 that provides for coupling with a light return fiber 4750. The optical fiber coupler 4760 can be, for example, a two-by-two fiber coupler. The light return fiber 750 can transmit reflected light to a detector 4775 (e.g., a light intensity meter, or an optical detector, such as an OCT detector). Light emitted by the light source 4730 can pass through the coupler 4760 and be transmitted into an optical fiber connector 4780, for example, a connector mounted at a surface of the optical system box 4700.

A flexible elongate instrument 4790 (e.g., a monorail catheter, a guidewire) can include an optical fiber connector 4785 at its proximal end that can be detachably connected to the optical connector 4780. The flexible elongate instrument 4790 includes a built-in or attached optical fiber (e.g., fiber 4670 in FIG. 36, fiber 2270 in FIG. 13A) that passes light from the connector 4785 to a distal portion 4765 of the instrument that includes an optical window (e.g., window 4640) that can function as an encoding detector sensor. Reflected light is collected by the optical window 640 and transmitted back through the fiber 4790, to the connector 4780, and to the optical fiber coupler 4760. Through the coupler 4760, at least a portion of the reflected light can be split and permitted to pass through the light return fiber 4750 into the detector 4775. The light collected at the detector 4775 can provide for a measured intensity signal and/or a depth profile signal, which can then be provided to data acquisition processor and converted to code characters.

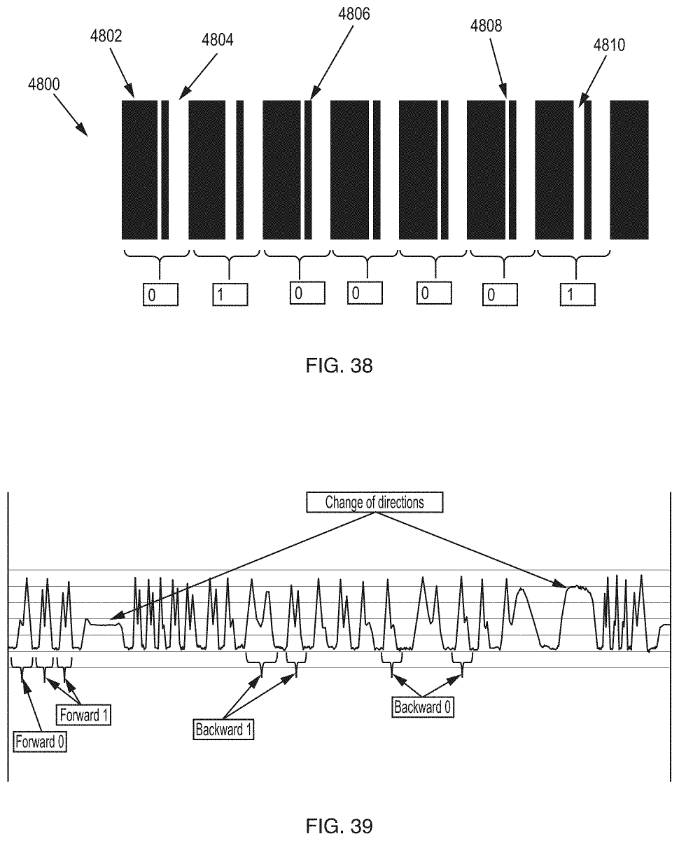

The example systems and methods shown in FIGS. 32-35 are illustrated as providing for four-bit position codes; however, a code track and encoding algorithm can provide for and make use of any number of bits for a given position code. Examples of six-bit or seven-bit position encoding that can also provide for direction determination are shown in FIGS. 38 and 39. A six-bit code can define up to 64 unique positions; and a seven-bit code can define up to 128 unique positions.

As illustrated in FIG. 38, an example code section 4800 includes several of each of the following: a black (or low reflectance) separator bar 4802; a white (or high reflectance) separator bar 4804; a black (or low reflectance) character bar 4806; and white (or high reflectance) character gaps 4808, 4810. As illustrated, the character gap 4808 defines a “0” character, and the character gap 4810 defines a “1” character. As illustrated, seven bits are provided, coding for “010001.” For a six-bit encoding, one less character can be provided.

A width of each of the bars 4802, 4804, 4806, 4808, 4810 can vary depending upon a size of the instrument on which the code is applied (e.g., by reflectance coating, engraving depth, etc.) and a size of an optical fiber/window for detection. For example, widths of each of the

The code bars 4802, 4804, 4806, 4808, 4810 can be of widths of about 20 μm to about 1000 μm. For example, for small fiber applications, widths can be from about 30 μm to about 200 μm, and for large fiber applications, widths can be about 50 μm to about 500 μm. In a large fiber application, for an example, the black separator bar 4802 can have a width of about 500 μm, the white separator bar 4804 can have a width of about 250 μm, the black character bar 4806 can have a width of about 100 μm, the character gap 4808 can have a width of about 56 μm, and the character gap 4810 can have a width of about 160 μm. For a small fiber application, for an example, the black separator bar 4802 can have a width of about 170 μm, the white separator bar 4804 can have a width of about 105 μm, the black character bar 4806 can have a width of about 42 μm, the character gap 4808 can have a width of about 32 μm, and the character gap 4810 can have a width of about 68 μm.

An example signal produced from an encoding as defined using the example configuration shown in FIG. 38 is shown in FIG. 39. As is visible in the figure, forward “0” and forward “1” are distinguishable from backward “1” and backward “0,” and changes in direction of movement are clearly detectable.

### 5. DEFINITIONS AND EXAMPLES

As used herein, the term “patient” or “patient in need thereof” refers to humans as well as non-human animals, such as domesticated mammals, including, without limitation, pigs, cats, dogs, and horses. The systems and methods provided are not limited to the imaging of humans and are applicable to veterinary imaging as well.

As used herein, the term “body lumen” refers to an inside space of a tubular or cavity structure within a patient. For example, a body lumen can be an artery, vein, or capillary in which blood flows (also referred to as “blood vessel”). A body lumen can be a colon, cranial vasculature, uterus, womb, lung, tracheal tract, ear canal, bladder, urethral tract, or uterine tract.

As used herein, the term “distal end” of a component or of a device is to be understood as meaning the end furthest from the user's hand (e.g., a physician administering a PCI) and the “proximal end” is to be understood as meaning the end closest to the user's hand. Likewise, in this application, the “distal direction” is to be understood as meaning the direction of insertion, and the “proximal direction” is to be understood as meaning the opposite direction to the direction of insertion.

As used herein, the term “flexible elongate instrument” refers to a medical instrument adapted for use inside of a body lumen through access via small puncture through the skin and tissue or via an orifice. The medical instrument is often elongated to impart flexibility and can optionally be lubricious for enabling access deep into a body lumen. More than one flexible elongate instrument can be used for an endoluminal procedure, in which case the plurality of flexible elongate instruments are herein referred to as a first flexible elongate instrument and a second flexible elongate instrument.

Terms such as “first” and “second”, and other numerical terms, when used herein, do not imply a sequence or order unless clearly indicated by the context. For example, a “first flexible elongate instrument” and a “second flexible elongate instrument” do not intend to refer to one flexible elongate instrument being inserted into the body lumen before, or primary to, another flexible elongate instrument.

Flexible elongate instruments, alternatively referred to herein as “flexible elongate endoluminal instruments,” can be adapted for navigation inside of a body lumen to access a target location. A flexible elongate instrument can be a guidewire and/or can comprise a section which performs a therapeutic and/or diagnostic function while inside of the body lumen. For example, at least two flexible elongate instruments can be used in an endoluminal procedure, with a first flexible elongate instrument being a guidewire or catheter, and a second flexible elongate instrument being a diagnostic and/or therapeutic device or a catheter of a diagnostic/therapeutic device. In a further example, when two or more flexible elongate instruments are used in an endoluminal procedure, the first flexible elongate instrument can comprise an orifice through which the second flexible elongate instrument can traverse. The first flexible elongate instrument can comprise a central axis, and a central axis of a second flexible elongate instrument can travel in parallel or about parallel to the central axis of the first elongate instrument, for example, while a portion or all of the first flexible elongate instrument is positioned inside of a body lumen. Where a flexible elongate instrument is a catheter, it can further comprise at least one inner lumen to travel over and parallel to another flexible elongate instrument, such as a guidewire.

A flexible elongate instrument can be a guidewire comprising a sensor and a plurality of radiopaque markers. The sensor can be a location information sensor, such as a sensor that detects one or a plurality of displacement encoding markers on another device and/or a sensor that detects a signal from a diagnostic device for co-location position determination. The functional modality of the sensor can be optical, magnetic, or capacitive in nature.

In an example configuration, a first flexible elongate instrument (e.g., a guidewire) comprises a sensor that acts as a marker encoding reader, and a second flexible elongate instrument (e.g., a catheter) comprises displacement encoding markers (e.g., engraved markings disposed on the second flexible elongate instrument interfacing with the sensor as the second flexible elongate instrument traverses along the guidewire, or heat-shrinkable tubing through which the second flexible elongate instrument is inserted followed by application of heat sufficient to shrink said tubing) When the second flexible elongate instrument is moving parallel to the first flexible elongate instrument, a relative displacement of the first flexible elongate instrument and the second flexible elongate instrument can be measured from the sensor reading the displacement encoding markers. When a plurality of radiopaque markers is disposed on the first flexible elongate instrument (e.g., a guidewire), the radiopaque markers can serve as a reference coordinate system, as detected by the X-ray angiography image, such that a position of the second flexible elongate instrument to the coordinate system can be measured in real-time or about real-time. The second flexible elongate instrument can be a therapeutic and/or diagnostic device.

A flexible elongate instrument can comprise, at least in part, one or more rigid portions or components. For example, a flexible elongate instrument can include or provide for the travel of a biopsy device or an aspiration device, which can include a rigid needle or other rigid structure(s) to effect obtaining a diagnostic sample or providing for delivery of a treatment.

As used herein, the term, “therapeutic and/or diagnostic device” refers to a region of a flexible elongate endoluminal instrument that is adapted to perform a function when inside of a body lumen. Examples of therapeutic and/or diagnostic devices on a flexible elongate endoluminal instrument include a stent, balloon, ablation tips, electrodes, ultrasound imaging transducer, pressure sensor, and optical coherent tomography light emitting tip.

As used herein, the terms “diagnostic device” or “diagnostic system” refers to medical equipment, medical systems, an instrument or a component thereof, an apparatus or substance, either active or passive, that is used during medical procedures, including interventional procedures both inside and/or outside of the body, for the detection, analysis, and/or measuring of a disease or medical condition of a patient. A diagnostic device can, for example, measure a temperature, pressure, conductivity, density, blood flow rate, oxygen level, or tissue morphology of the lumen. Examples of diagnostic devices that can be used with the provided methods and systems include intravascular ultrasound (IVUS) devices, optical coherence tomography (OCT) devices, photoacoustic sensing devices, fractional flow reserve (FFR) devices, endoscopic devices, arthroscopic devices, biopsy devices, and other devices which include a sensor configured to measure a tissue composition, a physical property, a physiological property, and/or a molecular property of anatomy.

As used herein, the terms “therapeutic device” of “therapeutic system” refers to medical equipment, medical systems, an instrument or a component thereof, an apparatus or substance, either active or passive, that is used during medical procedures, including interventional procedures for the treatment of a disease or medical condition of a patient, and in the prevention of disease or condition, amelioration from a disease or condition, or maintenance or restoration of health. Examples of therapeutic devices that can be used with the provided methods and systems include angioplasty devices, stents, embolization devices, atherectomy devices, ablation devices, drug-delivery devices, optical delivery devices, aspiration devices, and other devices capable of delivering a mechanical or physical intervention, a chemical intervention, or an energy-delivery intervention.

A therapeutic and/or diagnostic device can comprise one or a plurality of sensors. The sensor can be an ultrasound transducer (for IVUS), an optical light emitter/receiver (for OCT), a pressure sensor (for FFR). The sensor can be configured to be a component of (e.g., by mounting or affixing to) a flexible elongate instrument (e.g., a catheter or a guidewire). A therapeutic and/or diagnostic device can include or exclude: IVUS, OCT, FFR, or iFR.

Examples of IVUS imaging instruments suitable for use with the systems and methods described herein include: Boston Scientific Polaris, Phillips (Volcano) S5, Phillips S5i, Phillips CORE Mobile, Phillips SyncVision, Phillips IntraSight, and ACIST HDi. Examples of OCT imaging instruments for use with the systems and methods described herein include: Abbott (St. Jude) OPTIS, Terumo Lunawave, and Terumo FastView. Cardiology imaging instruments, with which the methods described herein can be performed, can include or exclude any of the foregoing OCT or IVUS imaging instruments, and the following: Boston Scientific Avvigo, Abbott Radianalyzer Xpress, Abbott QUANTIEN, Abbott Pressure Wire Receiver, ACIST RXI, OpSens Optowire and Conavi Novasight Hybrid System.

A diagnostic and/or therapeutic device can be a guidewire, a microcatheter, a thrombectomy catheter, a steerable catheter, a balloon catheter, a device delivery catheter, a cardiac catheter, a renal catheter, an urinary catheter, an oncology catheter, a robotic catheter/guidewire, a biopsy device, an atherectomy device (which can include or exclude an aperipheral arterial disease catheter), a lithotripsy device, or a neuromodulation device. A cardiac catheter can include or exclude a radiofrequency ablation catheter, a mapping catheter, a percutaneous transluminal angioplasty (PTA) catheter, an embolic protection device, a chronic total occlusion device, an infusion catheter, a snare, a support catheter, a thermodilution catheter, and a valvulotome. A diagnostic and/or therapeutic device can be configured to be used in a body lumen which does or does not have blood flow.

As used herein, the terms “diagnostic scan” or “body lumen information scan” or “vessel displacement scan” refer to imaging or assessing part or all of a body lumen using a diagnostic device. A diagnostic scan can measure any of a pressure, temperature, density, conductivity, inductance, tissue morphology, etc. at selected locations across the body lumen.

As used herein, the term “radiopaque” refers to refers to opacity from the radio wave to X-ray portion of the electromagnetic spectrum. Radiopaque components serve as a contrast when viewed with X-rays. Radiopaque materials can be made from, for example, titanium, platinum, gold, palladium, tungsten, barium, zirconium oxide, or any material identified by ASTM F640 Standard Test Methods for Measuring Radiopacity for Medical Use, as of Oct. 1, 2020.

As used herein, the term “IVUS” refers a method of imaging tissue using intravascular ultrasound. IVUS methods can include use of a device comprising an ultrasound probe attached at a distal end of a therapeutic and/or diagnostic device. A proximal end of the therapeutic and/or diagnostic device can be connected (either by wire, or wirelessly) to a computer. X-ray angiography is used to visualize a body lumen from external body and to guide physicians to navigate an IVUS catheter moving along a guidewire and imaging inside from a body lumen. IVUS data analysis methods are described, for example, in U.S. Pat. Nos. 4,794,931, 5,000,185, and 5,313,949; 5,243,988, and 5,353,798; 4,951,677; 5,095,911, 4,841,977, 5,373,849, 5,176,141, 5,240,003, 5,375,602, 5,373,845, 5,453,575, and 5,135,486, the teachings of which are incorporated herein by reference.

An IVUS catheter can move along a flexible elongate instrument that comprises a transducer, and the flexible elongate instrument can send distance signal information to a calculation unit to generate displacement information. The flexible elongate instrument can send distance signal information to the IVUS system to generate displacement information, when the IVUS system comprises or interfaces to the calculation unit. In IVUS methods, a second flexible elongate instrument can be a catheter comprising a plurality of radiopaque markers and a sensor. The catheter can be an IVUS catheter that can move inside the body lumen. The IVUS catheter can further comprise a motor-drive connected to the proximal end of the catheter, outside of the patient's body. Before performing IVUS catheter pullback, an operator/physician can take an X-ray image that captures both the body lumen and the radiopaque markers inside the body lumen, thereby establishing a relationship between the plurality of radiopaque markers in reference to the body lumen image as captured by the X-ray. During pullback, the IVUS transducer travel distance can be measured by the motor-drive pullback device on catheter's proximal end, outside body. The motor-drive position can determine a location of the IVUS transducer, and a relationship of the IVUS transducer location to the imaging markers can be established based on the transducer and imaging markers being disposed on the same catheter at known distances. The travel displacement from the motor drive unit can be input to the calculation unit to locate the position of the IVUS transducer during the pullback scan with the captured X-ray image containing the plurality of imaging markers as a reference. Optionally, X-ray imaging can be applied at the beginning of the procedure, then turned off after acquisition of one X-ray image comprising the profile of the plurality of radiopaque markers. The second flexible elongate instrument can be an IVUS catheter attached to a robotic arm. The second flexible elongate instrument can be selected from: IVUS, OCT, a therapeutic catheter (which can include or exclude antrectomy or Intuitive Surgical's surgical arm or lung probe or Siemens's Corindus vascular robotic platform). The displacement can be measured by a motor-drive on a robotic system connected to the second flexible elongate instrument.

As used herein, the term “OCT” (optical coherent tomography) refers to a medical imaging method using a light-emitting probe that is configured to acquire three-dimensional images (e.g., at micron-resolution) from within an optical scattering media (e.g., biological tissue). Generally, OCT methods involve a light source that delivers a beam of light to an imaging device to image target tissue. The OCT light source can be selected from a broad spectrum of wavelengths, or provide a limited spectrum of wavelengths (e.g., near-infrared light). An OCT light source can be applied in pulsed durations or as a continuous wave. Examples of suitable OCT light sources include a diode, a diode array, a semiconductor laser, an ultrashort pulsed laser, and a supercontinuum light source. The OCT light source can be filtered and an OCT system can optionally allow an operator to select a wavelength of light to be amplified. Wavelengths commonly used in medical applications include near-infrared light for tissue penetrance, for example between about 800 nm and about 1700 nm. OCT systems and methods include those described in U.S. Pat. Nos. 8,108,030, 8,989,849, 8,531,676, 10,219,780, 8,125,648, 7,929,148, 7,474,407, U.S. Pat. No. U.S. Pat. Nos. 5,321,501, and 9,046,339, the teachings of which are incorporated herein by reference.

As used herein, the term “angiography” refers to a medical imaging method that involves a combination of X-ray angiography imaging, typically fluoroscopy, and radiopaque contrast agent injections into the patient to identify a structure of the patient's vasculature. Real-time vasculature images can be displayed on a monitor during a PCI procedure such that the operator/physician can view the manipulation of the guidewire or inserted device in real-time or with minimal lag time. A displayed image (i.e., angiogram) can be processed with software and displayed on a computer, or the image may be a closed-circuit image of a scintillating surface combined with a visibly fluorescent material.

As used herein, the term “FFR” or “fractional flow reserve” refers to its meaning in the art and includes a method to measure a blood pressure difference across a body lumen, wherein the body lumen is a coronary artery. The blood pressure difference can result from stenosis. FFR methods typically involve use of a flexible elongate instrument comprising a pressure transducer to measure pressure, temperature, and/or blood flow. FFR is typically performed when the patient is induced to have maximal blood flow (hyperemia). Maximal blood flow can be achieved by administering a vasodilator to the patient. The flexible elongate instrument is pulled back (e.g., as in a “pullback” scan), and pressures are recorded across the body lumen. FFR can be measured as a ratio of maximum blood flow distal (p_d) to a stenotic lesion to normal maximum flow (p_a) in the blood vessel, as provided by: FFR={p_{d}}/{p_{a}}.

As used herein, the term “iFR” or “instantaneous wave-free ratio” refers to its meaning in the art and includes a method to measure blood pressure difference across a body lumen. The body lumen can be a coronary artery and the method does not require the administration of a vasodilator to the patient. In iFR, a flexible elongate instrument comprising a pressure transducer is positioned to a point distal to a stenotic lesion. During a period of diastole known as the “wave-free period,” iFR then calculates the ratio of the distal coronary artery pressure (Pd) to the pressure within the aortic outflow tract (Pa). During this timeframe completing blood flow complicating these measurements is negligible.

As used herein, the term “stent” refers to a tubing placed into a body lumen to keep a passageway open. Stents can be placed into, for example, a coronary lumen to treat a coronary disease, a cerebrovasculature lumen to treat a cerebrovascular disease, a peripheral lumen to treat a peripheral disease, a ureteral lumen to treat an ureteral disease, and a gastrointestinal lumen to treat a gastrointestinal tract disease.

As used herein, the term “real-time or about real-time” means the occurrence of an event at the present time or delayed by some amount of time due to latency in the circuitry of the system components. An about real-time event is one that would be in real time but for the delay in transfer of data, either electronically or wirelessly. A delay in transfer of data can range from, for example, 1 nanosecond to 1 second, including any time period in between.

A position of a sensor relative to a plurality of displacement encoding markers can be measured by a function of time and speed at which the sensor moves relative to the plurality of encoding markers.

As used herein, the term, “linear position” refers to a distance between two objects or two identified regions in a body lumen, as measured following the path of the body lumen. The shape of the line can thus be straight or curvilinear. A curvilinear line can comprise multiple curves. The term “linear position” is used to distinguish from the term “linear distance” which refers to a distance between the two selected objects or two identified regions.

As used herein, the term “body lumen reference point” refers to a body lumen location that coincides with a location on a flexible elongated instrument having a plurality of imaging markers and positioned inside of the body lumen when an external body or angiographic image of the body lumen and flexible elongated instrument is obtained. The location on the flexible elongated instrument has known distances to the plurality of imaging markers. This location can coincide with an imaging marker itself (e.g., when a diagnostic sensor is on the same flexible elongated instrument as the plurality of image markers, and therefore the diagnostic sensor location is known relative to the imaging markers, as in the example shown in FIG. 4; or when the diagnostic sensor and plurality of imaging markers are on different flexible elongated instruments, but the diagnostic sensor location relative to the image markers is determined from an angiographic image, as in the example shown in FIGS. 5A-5B). Alternatively, the location can be the location of a signal transducer used to determine when another transducer, such as a diagnostic sensor, is next to or coincident with it, as in the example shown in FIG. 6

As used herein, the term “body lumen diagnostic scan” refers to a scan performed by body lumen diagnostic sensor that obtains body lumen information while displacing inside of a body lumen. The obtained body lumen information can be correlated with a measured displacement.

As used herein, the term, “imaging marker” refers to a segment of finite length located on a flexible elongated instrument that is visually distinguishable from “no marker” sections when viewed by an external body imager. An example of an imaging maker on a catheter or a guidewire for an X-ray imager is a radiopaque marker made of a heavy element that blocks more X-ray than the native catheter or guidewire material. An imaging marker can be detectable by both an X-ray angiogram and a diagnostic sensor, at either the same time or at different times. An imaging marker can be MR and/or NMR-sensitive (e.g., comprises atoms with a free nuclear spin), electromagnetic sensitive, electromechanical sensitive, optically sensitive, and/or mechanically sensitive. An imaging marker can be ultrasound-sensitive (e.g., comprising bands filled with an agent having a different acoustic impedance from that of human blood). An imaging marker can be detectable in one or more imaging modalities. For example, imaging markers can comprise nanoparticles that enable visibility under MM and fluoroscopy (e.g., EmeryGlide™ wire (B. Braun Interventional Systems Inc.)).

A plurality of imaging markers (e.g., radiopaque markers) on a first flexible elongate instrument can serve as the basis of a coordinate system to quantify a position of a target (e.g., a sensor disposed on second flexible elongate instrument). Once a displacement or movement (delta x) of a target relative to the plurality of imaging markers is detected by a displacement calculation mechanism, a position of the target can be established in the coordinate system. The displacement calculation mechanism can be based upon pullback time, the reading of encoding markers, or from a combination thereof.

As used herein, the term “displacement” refers to an absolute value that started from zero, using a reference position as zero. The reference position can serve as the basis of a 2D or 3D reference coordinate system for determining subsequent positions of one or more flexible elongate instruments. A displacement can be calculated, for example, from displacement encoding markers using the following formula: Location=Displacement+Offset. The offset is the distance from a starting point of the device to a selected displacement encoding marker or to a point between encoding markers. Alternatively, or in addition, a displacement can be measured relative to the reference position by calculating pullback speed(s) based on pullback timestamps. In IVUS methods, a typical pullback rate can vary between 0.5 to 1 mm per second. In OCT methods, a typical pullback speed is 20 mm per second, with a pullback length of about 50 mm. As a non-limiting example, when the pullback speed is 1 mm per second and the pullback is performed for 50 seconds, the displacement distance can be calculated to be about 50 mm.

A diagnostic device (e.g., flexible elongate instrument having a diagnostic device) can comprise a displacement sensor, displacement encoding markers, or both. Separately, a guidewire can comprise displacement encoding markers, a displacement sensor, or both. The displacement sensor can detect a relative movement of one or a plurality of encoding markers relative to the sensor or a distance the sensor has traversed along a flexible elongate instrument relative to a reference position such that displacement can be measured. The sensor can be an optical sensor, an electrical sensor, an electromagnetic senor, a mechanical sensor, a pressure sensor, a chemically-selective sensor, and/or a sonographic sensor. A displacement sensor can optionally detect relative positions of encoding markers. The sensor can be, for example, a transducer selected to transmit and/or receive electromagnetic (e.g., inductance, resistance, voltage), light, ultrasound, or pressure signals.

In systems and methods in which displacement is measured using encoding markers, the encoding markers can be configured to be on a sleeve that is separate from the flexible elongate instrument. The sleeve can be positioned parallel to, or sharing about the center of axis of, a flexible elongate instrument and can be configured to travel along the length of the flexible elongate instrument. For example, the sleeve can comprise a heat-shrinkable tubing, such that the sleeve will shrink around a flexible elongate instrument upon the application of heat. A shape of the displacement encoding elements about the sleeve can be a “zig-zag” pattern, such that when the heat-shrinkable tubing is heated, the “zig-zag” periodicity is reduced, but the encoding element is of a greater density per unit area about the flexible elongate instrument. Alternatively, encoding markers can be configured to be on the flexible elongate instrument, and surrounded by a sleeve.

Flexible elongate instruments can generally comprise a proximal end, a distal end, and at least one of a sensor and a plurality of elements circumferentially or partially circumferential positioned around the flexible elongate instrument. A sensor disposed on or in a flexible elongate instrument can be shaped and adapted for insertion into a body lumen. The elements can be imaging markers, displacement encoding markers, or both. The plurality of elements can be independently of a selected distance from each other, of a selected dimension (e.g., width), and/or of a selected shape. The width of the elements can range from 0.01 mm to 3 cm. The number of elements can range from 2 to 500. The number of elements can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. Optionally, the elements can provide for a checksum—for example, a width of three successive elements can equal a width of a fourth successive element.

As used herein, the term, “displacement encoding” refers to a region on a flexible elongated instrument that comprises a plurality of encoding elements (also referred to as “encoding markers”) positioned at selected distance intervals on the flexible elongate instrument. The encoding elements are detectable by an encoding sensor.

As used herein, the term “encoding sensor” refers to a device that can detect or measure the displacement encoding. The displacement encoding can be positioned to be located on a first flexible elongate instrument and the encoding sensor can be positioned to be located on a second flexible elongate instrument. When the encoding sensor is in proximity to the displacement encoding, the encoding sensor can detect one or a plurality of the encoding elements. The encoding sensor can, for example, comprise a transducer that can transmit and/or receive a physical signal. The physical signal can be optical, electrical, magnetic, inductive, or capacitive. Variations in the signal generated by the encoding sensor on a second flexible elongate instrument when the encoding sensor is in proximity to, and moving in a direction parallel to, the first flexible elongate instrument, can be used to measure the relative displacement of the encoding sensor on the second flexible elongate instrument relative to the encoded section on a first flexible elongate instrument.

### 6. COMPUTER IMPLEMENTED SYSTEMS

The systems and methods provided herein are generally useful for predicting the location of diagnostic and/or therapeutic devices within a body lumen. The methods can be implemented on a computer server accessible over one or more computer networks. In some embodiments, the one or more computer networks can interface with a computer server. The computer server where the methods are implemented may in principle be any computing system or architecture capable of performing the computations and storing the necessary data. The exact specifications of such a system can change with the growth and pace of technology, so the example computer systems and components described herein should not be seen as limiting. The systems will typically contain storage space, memory, one or more processors, and one or more input/output devices. It is to be appreciated that the term “processor” as used herein is intended to include any processing device, such as, for example, one that includes a CPU (central processing unit). The term “memory” as used herein is intended to include memory associated with a processor or CPU, such as, for example, RAM, ROM, etc. In addition, the term “input/output devices” or “I/O devices” as used herein is intended to include, for example, one or more input devices, e.g., keyboard, for making queries and/or inputting data to the processing unit, and/or one or more output devices, e.g., a display and/or printer, for presenting query results and/or other results associated with the processing unit. An I/O device might also be a connection to the network where queries are received from and results are directed to one or more client computers. It is also to be understood that the term “processor” may refer to more than one processing device. Other processing devices, either on a computer cluster or in a multi-processor computer server, may share the elements associated with the processing device. Accordingly, software components including instructions or code for performing the methodologies of the invention, as described herein, may be stored in one or more of the associated memory or storage devices (e.g., ROM, fixed or removable memory) and, when ready to be utilized, loaded in part or in whole into memory (e.g., into RAM) and executed by a CPU. The storage may be further utilized for storing program codes, databases of genomic sequences, etc. The storage can be any suitable form of computer storage including traditional hard-disk drives, solid-state drives, or ultrafast disk arrays. In some embodiments the storage includes network-attached storage that may be operatively connected to multiple similar computer servers that comprise a computing cluster.

The data can be real-time or about real-time, and/or data acquired from different procedures and/or steps such as but not limit to ECG (Electrocardiogram), Doppler, FFR (Fractional flow reserve), FFR-CT (Fractional flow reserve-computed tomography), IVUS (intravascular ultrasound), and OCT (Optical coherence tomography). The computer system can also save raw data and final processed data from memory banks to local and/or external storage system for further data processing from other therapeutic and/or diagnostic instruments.

The systems and methods of the invention can be applied to any interventional procedures for a body lumen. The body lumen can include or exclude: blood vessels, vasculature of the lymphatic and nervous systems, various structures of the gastrointestinal tract including lumen of the small intestine, large intestine, stomach, esophagus, colon, pancreatic duct, bile duct, hepatic duct, lumen of the reproductive tract including the vas deferens, uterus and fallopian tubes, structures of the urinary tract including urinary collecting ducts, renal tubules, ureter, and bladder, and structures of the head and neck and pulmonary system including sinuses, parotid, trachea, bronchi, and lungs.

The methods described herein can be performed on a computer, which may include or exclude non-transient memory comprising a set of instructions for performing the methods. The systems described herein can comprise a computer and at least one non-transitory machine-readable medium storing instructions which, when executed by a programmable processor, cause the programmable processor to perform operations comprising a selected method as described herein.

The computer systems of this disclosure can comprise a visualization display. The form of the visualization display systems can vary such as but not limits to monitor, mobile device, wearable device and AR/VR head mounting device. The inputs from an operator/physician at are executed via an electronic device such as a computer, a server with a monitor, a host workstation, a controller with a monitor, or a third party system operator/physician interface. In some embodiments, the displays can comprise a 2D depiction of the body lumen comprising a flexible elongate instrument using voxels.

The equations and methods described herein can be performed on a computer processor. Processors suitable for the execution of computer program which include the equations and methods described herein can include or exclude a general purpose computer microprocessor, a special purpose microprocessors, and combinations thereof. A processor will receive instructions and data from a read-only memory or a random access memory or both. A computer comprises a processor for executing instructions and one or more memory devices for storing instructions and data. In some embodiments, the computer will also comprise, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, NAND-based flash memory, solid state drive (SSD), and other flash memory devices); magnetic disks, (e.g., internal hard disks or removable disks); magneto-optical disks; and optical disks (e.g., CD and DVD disks). In some embodiments, the processor and the memory can be supplemented by, or incorporated in, special purpose logic circuitry.

The computer can further comprise an I/O (input-output) device for enabling interaction with an operator/physician. In some embodiments, the I/O device can include or exclude a CRT, LCD, LED, or projection device for displaying information to the operator/physician, and an input or output device such as a keyboard and a pointing device, (e.g., a mouse or a trackball, Virtual Reality goggles, wearable touchpad and finger mounted pointing devices), by which the operator/physician can provide input to the computer. In some embodiments, the I/O device can transmit information to the computer from the operator/physician via sensory feedback, (e.g., visual feedback, auditory feedback, or tactile feedback), and input from the operator/physician can be received in any form, including acoustic, speech, or tactile input. In some embodiments the calculation unit can be connected to the display, input-output device, or both by a method selected from electronic connection or wireless connection. The wireless connection can be Bluetooth (a wireless technology standard used for exchanging data between fixed and mobile devices over short distances using UHF radio waves in the industrial, scientific and medical radio bands, from 2.402 GHz to 2.480 GHz), WiFi (IEEE 802.11 standard), or a cellular network such as 3G, 4G, 5G, or combinations thereof.

The computer described herein can further comprise a computing system that further comprises a back-end component (e.g., a data server), a middleware component (e.g., an application server), a front-end component (e.g., a client computer having a graphical operator/physician interface, or a web browser through which a physician/operator can interact with an implementation of the patient matter described herein), or any combination thereof. In some embodiments, the components of the computer system can be interconnected through a network by any form or medium of digital data communication, e.g., a communication network. In some embodiments, the communication network can include or exclude: cell networks (3G, 4G, 5G), Personal Area Network (wireless such as infrared, ZigBee, Bluetooth and ultrawideband, or UWB, and wired connection such as USB or FireWire), a local area network (LAN such as Ethernet (IEEE 802.3) and Wi-Fi/WLAN (IEEE 802.11)), and a wide area network (WAN), e.g., the Internet.

The equations and methods described herein can be performed on a computer system which further comprises or more computer programs tangibly embodied in an information carrier (e.g., in a non-transitory computer-readable medium) for execution by, or to control the operation of, data processing apparatus (e.g., a programmable processor, a computer, or multiple computers). In some embodiments, the computer program (also referred to as a program, software, software application, app, macro, or code) can be written in any form of programming language, including compiled or interpreted languages (e.g., C, C++, Perl, Machine Language, Assembly, C#, Python, MatLab), and it can be deployed in any form, including as a stand-alone program or as a module, component, subroutine, or other unit suitable for use in a computing environment. In some embodiments, the computer system can include programming language known in the art, including, without limitation, C, C++, C#, Perl, Java, ActiveX, HTML5, Visual Basic, Machine Language, Assembly, Python, MatLab, or JavaScript. In some embodiments, when using the C++programming language, the computer program can include or exclude the following tools: powerful Visualization Tool Kit (VTK) library for volumetric data visualization (https://www.vtk.org/), Insight Segmentation and Registration Toolkit (ITK) for implementation of different algorithms for medical volume segmentation (https://itk.org/), Qt—library for GUI (https://www.qt.io/), Common Tool Kit (CTK) for operator/physician interaction elements for use with VTK and CTK (http://www.commontk.org/index.php/Main_Page), Grassroots DICOM (GDCM) library to work with DICOM files, (https://sourceforge.net/projects/gdcm/), Boost, for type safe dimensional analysis for using information about measurement units. All of the aforementioned websites are as of Nov. 1, 2020 (confirmable by the Wayback Machine).

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs, which can also be referred to programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In some embodiments, the computer program can be deployed to be executed on one computer or a plurality of computer or processing units at one site or distributed across multiple sites and interconnected by a communication network.

In some embodiments, the computer program used to perform the equations and methods described herein can further comprise writing a file. In some embodiments, a file can be a digital file, (e.g., stored on a hard drive, SSD, CD, or other tangible, non-transitory medium). A file can be sent from one device to another over the communication network as packets being sent from a server to a client.

Writing a file can comprise transforming a tangible, non-transitory computer-readable medium, for example, by adding, removing, or rearranging particles (e.g., with a net charge or dipole moment) into patterns of magnetization by read/write heads, the patterns then representing new collocations of information desired by, and useful to, the user. In some embodiments, writing involves a physical transformation of material in tangible, non-transitory computer readable media with certain properties such that magnetic read/write devices can then read the new and useful collocation of information. In some embodiments, writing a file comprises using flash memory such as NAND flash memory and storing information in an array of memory cells include floating-gate transistors. Methods of writing a file are well-known in the art and, for example, can be invoked automatically by a program from software or from a programming language.

Any of the electronic devices and/or components mentioned above in this system, with the associated interfaces, may be controlled and/or coordinated by operating system software, such as Windows OS (e.g. Windows XP, Windows 8, Windows 10, Windows Server, etc.), Windows CE, Mac OS, iOS, Android, Chrome OS, Unix, Linux, VxWorks, or other suitable operation systems. In other embodiment, the said electronics may be controlled by a proprietary operating system. Conventional operating systems control and schedule system processes for execution, perform memory management, provide file system, networking, I/O services, and provide an user interface functionality, such as a graphical user interface (GUI), among other systems and/or devices.

### 7. EXAMPLE EMBODIMENTS

A1. A system for measuring body lumen locations and displaying information obtained from a diagnostic device at each body lumen location, comprising: a computer processor configured to obtain body lumen location information and generate display information, and a display, wherein the computer processor is further configured to obtain at least one X-ray angiographic image of a body lumen comprising an inserted flexible elongate endoluminal instrument therein, and a plurality of imaging markers configured to be on the instrument, such that both the body lumen and one or a plurality of the imaging markers are detectable, optionally, wherein the computer processor is further configured to obtain body lumen diagnostic scan data, which comprises body lumen diagnostic information from a diagnostic device of at least one location which is defined by a selected distance from a start point, optionally, wherein the computer processor is further configured to obtain the position of at least one body lumen reference point defined by the plurality of imaging markers identified from the X-ray angiographic image of the body lumen, such that the linear distance between the reference point and two or more of the plurality of imaging markers are known, optionally, wherein the computer processor is further configured to obtain the location of the start point, which is the distance between the start point and the body lumen reference point, optionally, wherein the computer processor calculates the locations of the at least one diagnostic point, identifies the relative location of the at least one diagnostic point to the plurality of imaging markers, and displays the diagnostic locations and associated diagnostic information in reference to the plurality of imaging markers.

A2. The system of A1, wherein the computer processor is further configured to interface with a display.

A3. The system of A1, wherein the computer processor is further configured to display a plurality of imaging markers on an IVUS pullback distance scale.

A4. A system for measuring body lumen locations and displaying said locations with information obtained from a diagnostic device, comprising: a first flexible elongate endoluminal instrument configured to be positioned within in a body lumen wherein the flexible elongate endoluminal instrument comprises a plurality of imaging markers, a second flexible elongate endoluminal instrument comprising a diagnostic and/or therapeutic device configured to be positioned within the body lumen as the first flexible elongate endoluminal instrument and configured to traverse parallel to said first flexible elongate endoluminal instrument, wherein the relative displacement between the first and second flexible elongate instruments is measured, a location computer processor configured to obtain body lumen location information, and generate a display of diagnostic information from a diagnostic device, which interfaces to a display, wherein the location computer processor obtains the position of at least one reference point located on the first flexible elongate endoluminal instrument, such that the distance between the reference point and one or a plurality of imaging markers is known, optionally, a display, one or a plurality of X-ray angiographic images of the body lumen, wherein both the body lumen and a plurality of imaging markers are detectable within an X-ray angiographic image, thereby defining at least one body lumen reference point which is the body lumen point of the at least one reference point when the X-ray angiographic image was generated.

A5. The system of A4, wherein the plurality of imaging markers is positioned at the distal portion of the first flexible elongate endoluminal instrument and each of the imaging markers comprises a selected dimension and the distance between each markers are of a selected distance, and at least one imaging marker is uniquely identifiable.

A6. The system of A4, wherein the second flexible endoluminal elongate instrument is IVUS.

A7. The system of A4, wherein the location computer processor is further configured to interface to a displacement measurement unit.

A8. The system of A4, wherein the location computer processor is further configured to obtain at least one displacement of the diagnostic and/or therapeutic device as measured from a start point.

A9. The system of A8, wherein the location computer processor is further configured to obtain the location of the start point, which is the distance between the diagnostic sensor and the body lumen reference points at the start of a diagnostic scan.

A10. The system of A4, wherein the location computer processor is further configured to calculate the locations of the diagnostic and/or therapeutic device from the received one or plurality of imaging markers, and transmits to a display the locations and diagnostic information obtained from a diagnostic device in reference to the plurality of imaging markers.

A11. A system for measuring body lumen locations and displaying the locations and diagnostic information obtained from a diagnostic device, comprising: a first flexible elongate endoluminal instrument configured to be positioned within a body lumen comprising a plurality of imaging markers, an image generated by an X-ray angiography instrument comprising at least one of the imaging markers of the flexible elongate endoluminal instrument inserted into the body lumen, wherein both the body lumen and the plurality of imaging markers are detectable and the detectable imaging markers provide fixed points for a linear location reference system for the body lumen on the generated X-ray angiography image, and a location computer processor which is configured to receive body lumen location information, and transmit the body lumen information, the linear location reference, and the X-ray angiography image to a display, wherein the location computer processor obtains at least one body lumen location in reference to the plurality of imaging markers as shown on the X-ray angiography image.

A12. The system of A11, wherein the plurality of imaging markers is configured to be positioned at the distal portion of the flexible elongate instrument.

A13. The system of A11, wherein each of the plurality of imaging markers comprises a selected dimension, and have a selected distance separating each of the imaging markers.

A14. The system of A11, wherein at least one of the plurality of imaging markers is uniquely identifiable.

A15. The system of A14, wherein at least one of the plurality of imaging markers comprises a selected indicia.

A16. The system of A11, further comprising: a second flexible elongate instrument comprising a plurality of displacement encoding markers wherein the second flexible elongate instrument is inserted into the body lumen, wherein the second flexible elongate instrument is configured to traverse parallel to the central axis of the first flexible elongate instrument, and an interface to a displacement measurement component comprising an encoding sensor, which is configured to detect the displacement encoding markers of the second flexible elongate instrument when moving inside the body lumen, wherein the first flexible endolumen elongate instrument comprises a diagnostic and/or therapeutic device which is at a selected distance from a selected imaging marker, the distance between the two defining a first quantify body lumen location, wherein the displacement measurement component comprising an encoding sensor detects at least one of a plurality of displacement encoding markers from a start position, wherein the distance between the start position and the first quantified body lumen location is zero, and the location computer processor is configured to calculate the location of the at least one of a plurality of displacement encoding markers within the body lumen, and display the calculated locations.

A17. The system of A11, further comprising: a second flexible elongate instrument comprising a diagnostic and/or therapeutic device and a plurality of displacement encoding markers wherein the second flexible elongate instrument is within the body lumen, and the diagnostic and/or therapeutic device is configured to traverse within the body lumen, an interface to a displacement measurement component comprising an encoding sensor which is configured to measure the displacement encoding markers of the diagnostic and/or therapeutic device when moving inside the body lumen, wherein the second flexible elongate endoluminal instrument further comprises at least one of a plurality of imaging markers with a selected length and selected distance between each of the plurality of imaging markers wherein at least one imaging marker is at a selected distance from the diagnostic and/or therapeutic device, wherein from an X-ray angiography image, the distance between the diagnostic and/or therapeutic device and the plurality of imaging markers on the first flexible elongate endoluminal instrument defines a first quantified body lumen location, wherein the displacement measurement component comprising an encoding sensor detects at least one of a plurality of displacement encoding markers from a start position, wherein the distance between the start position and the first quantified body lumen location is zero, and the location computer processor calculates the body lumen location of the at least one of a plurality of displacement encoding markers, and transmits the calculated locations to a display.

A18. The system of A17, wherein the calculated locations and transmitted to and depicted on the display.

A19. The system of any of A16 or 17, wherein the diagnostic and/or therapeutic device comprises a diagnostic sensor configured to obtain body lumen information correlated to the at least one of a plurality of displacement encoding markers, and the system further comprises an interface receiving said generated body lumen information, and wherein the body lumen information is correlated to the calculated body lumen locations, and the correlated body lumen information to the calculated body lumen locations is transmitted to a display, and optionally further displayed.

A20. The system of A11, further comprising: a body lumen diagnostic sensor positioned on a flexible elongate instrument inserted into the body lumen, wherein the diagnostic body lumen diagnostic sensor is configured to traverse within the body lumen, a location computer processor interfaced to a displacement measurement component comprising an encoding sensor which is configured to measure the displacement encoding markers of the diagnostic sensor when moving inside the body lumen, wherein the location computer processor is further configured to detect a displacement measurement from a start position, wherein the diagnostic sensor detects body lumen information correlated to the at least one of a plurality of displacement encoding markers, and the location computer processor further comprises an interface which receives the generated body lumen information.

A21. The system of A20, wherein the diagnostic sensor is on a second flexible elongate endoluminal instrument.

A22. The system of A21, wherein the first flexible elongate endoluminal instrument further comprises a signal transducer operating at a detectible modality and range of the diagnostic sensor, positioned at a selected distance from one of the plurality of imaging markers, wherein said distance defines a first quantified body lumen location.

A23. The system of A22, wherein the signal transducer is interfaced to a signal unit capable of generating and optionally receiving signals.

A24. The system of A23, wherein based on the diagnostic sensor information generated while measuring displacement encoding markers from a starting position, the sensor measures a displacement position where the diagnostic sensor co-aligns with the signal transducer, wherein the distance between the start position and the first quantified body lumen location is the displacement of the diagnostic sensor at the co-alignment point.

A25. The system of A24, wherein the location computer processor is configured to calculate the body lumen location of the at least one of a plurality of displacement encoding markers, and the body lumen information is correlated to the calculated body lumen locations, and is optionally displayed.

A26. The system of A11, wherein the location computer processor further comprises an interface to the signal unit, wherein the co-alignment position between the diagnostic sensor and the signal transducer is measured based on signal received by the signal transducer.

A27. The system of A11, wherein the location computer processor further comprises an interface to the signal unit, wherein the co-alignment position between the diagnostic sensor and the signal transducer is measured based on timing between the emitted signal and received signal.

A28. The system of any of A11-27, wherein the generation and display of body lumen locations, and/or body lumen information in conjunction with the body lumen locations is real-time or about real-time as when the body lumen distances to a first body lumen point are received.

A29. The system of any of A11-28, wherein the diagnostic and/or therapeutic device comprises a defined dimension positioned in the body lumen, and the location computer processor further receive the defined dimensions of the diagnostic and/or therapeutic device, generating a graphical representation of the diagnostic and/or therapeutic device, and display in relation to the graphical representation of the plurality of imaging markers.

A30. A method for measuring and displaying body lumen locations and diagnostic information associated with said locations, comprising: inserting a flexible elongate endoluminal instrument comprising a distal end and a proximal end into a body lumen, wherein the flexible elongate endoluminal instrument further comprises a plurality of imaging markers at the distal portion of the instrument and each marker comprises a selected dimension and a selected distance between each imaging markers, wherein the flexible elongate endoluminal instrument further comprises a displacement measuring component located at a selected distance from the plurality of imaging markers, capable of generating body lumen information and adapted to displace inside of the body lumen, and the selected distance between the diagnostic displacement measuring component and the plurality of imaging markers is received by a location computer processor, wherein the displacement measuring component is configured to measure displacement encoding markers, obtaining at least one X-ray angiographic image of the body lumen with the inserted flexible elongate endoluminal instrument such that both the body lumen and the plurality of imaging markers are detectable, and at least one imaging marker is uniquely identifiable, performing a body lumen diagnostic scan from a start position where the diagnostic sensor is at the relative location to the plurality of imaging markers as where it is in the obtained X-ray angiography image, transmit the body lumen information and displacement measurements to a diagnostic processor, correlating the body lumen information to measured displacement encoding markers, transmitting the correlated displacement encoding markers and body lumen information to a location computer processor, wherein the location computer processor is configured to measure the location of each received displacement point by calculating their distances to the plurality of imaging markers, and correlate the body lumen information with the locations, and output the correlated body lumen information with the locations to a display, and optionally displaying the information.

A31. A method for measuring and displaying body lumen locations and diagnostic information associated with said locations, comprising: inserting a first flexible elongate endoluminal instrument comprising a distal end and a proximal end into a body lumen, wherein the first flexible elongate endoluminal instrument comprises a plurality of imaging markers at the distal portion of the instrument and each marker dimension is of a selected length and the distances between each of the imaging markers are of a selected distance, inserting a second flexible elongate endoluminal instrument comprising at least one body lumen diagnostic sensor capable of obtaining body lumen information, and a plurality of imaging markers located at a selected distance from the at least one body lumen diagnostic sensor, wherein the displacement of the encoding markers to the diagnostic sensor is measured by a displacement measuring component, obtaining at least one X-ray angiogram of the body lumen with the inserted flexible elongate endoluminal instruments such that both the body lumen and the plurality of imaging markers from both the first and second flexible elongate endoluminal instrument are detectable, and at least one imaging marker on the first flexible elongate endoluminal instrument is uniquely identifiable, measuring the distance between the diagnostic sensor and the plurality of imaging markers on the first flexible elongate endoluminal instrument from on the plurality of imaging markers positions on both flexible elongate endoluminal instruments detected by the X-ray angiographic image, and output the measured distance to a location computer processor, performing a body lumen diagnostic scan from a start position where the diagnostic sensor is at the relative location to the plurality of imaging markers on the first instrument as where it is in the obtained X-ray angiography image, output the body lumen information and displacement measurements to a diagnostic processor, wherein the body lumen information is correlated to measured displacement encoding markers, and transmitting the correlated displacement encoding markers and body lumen information from the diagnostic processor to the location computer processor, wherein the location computer processor is configured to measure the location of each received displacement point by calculating their distances to the plurality of imaging markers, and correlate the body lumen information with the locations, and output the correlated body lumen information with the locations to a display.

A32. A method for measuring and displaying body lumen locations and diagnostic information associated with the locations, comprising: inserting a first flexible elongate endoluminal instrument comprising a distal end and a proximal end into a body lumen, wherein the first flexible elongate endoluminal instrument comprises a plurality of imaging markers at the distal portion of the instrument and each marker dimension is of a selected width and the distances between each of the imaging markers are of a selected distance, wherein the first flexible elongate endoluminal instrument comprises a signal transducer located at a selected distance from the plurality of imaging markers, and the selected distance is received by a location computer processor, and the signal transducer is interfaced to a signal unit, inserting a second flexible elongate endoluminal instrument comprising at least one body lumen diagnostic sensor capable of generating body lumen information and adapted to displace inside of the body lumen, wherein displacement between the body lumen diagnostic sensor and the imaging markers is known or measured by a displacement measuring component, wherein the signal transducer located on the first flexible elongate endoluminal instrument operates at a detectible modality and range of the diagnostic sensor located on the second flexible elongate endoluminal instrument, obtaining at least one X-ray angiogram of the body lumen with the inserted flexible elongate endoluminal instruments such that both the body lumen and at least one of the plurality of imaging markers from the first flexible elongate endoluminal instrument are detectable, and at least one imaging marker is uniquely identifiable, performing a body lumen diagnostic scan from a start position, output the body lumen information and displacement measurements to a diagnostic processor, wherein the body lumen information is correlated to measured displacement encoding markers, measure a co-alignment distance defined by the displacement of the diagnostic sensor from the start position to the position when it is co-aligned with the signal transducer, transmitting the co-alignment distance to the location computer processor, and transmitting the correlated displacement and body lumen information from the diagnostic processor to the location computer processor, wherein the location computer processor is configured to measure the location of each received displacement point by calculating their distances to the plurality of imaging markers, and correlate the body lumen information with the locations, and output the correlated body lumen information with the locations to a display, and optionally displaying the information.

A33. The method of A32, wherein the location computer processor further comprises an interface to the signal unit, wherein the co-alignment position between the diagnostic sensor and the signal transducer is measured based on signal received by the signal transducer.

A34. The method of A32, wherein the location computer processor further comprises an interface to the signal unit, wherein the co-alignment position between the diagnostic sensor and the signal transducer is measured based on timing between the emitted signal and received signal.

A35. The method of A32, wherein the flexible elongate endoluminal instrument that comprises the plurality of imaging markers is a medical guidewire.

A36. The method of any of A32-35, wherein the generation and display of body lumen locations, and/or body lumen information with the body lumen locations is performed in real-time or about real-time when the body lumen diagnostic scan is performed.

A37. A system for identifying the locations of imaging markers on both a diagnostic imaging display and an X-ray angiogram display, comprising: one or a plurality of flexible elongate endoluminal instrument configured to be inserted in a body lumen that comprises a plurality of imaging markers positioned at the distal end of the instrument and each imaging marker dimension is of a selected width and distances between each of the plurality markers are of a selected distance, an X-ray angiogram comprising an image of a body lumen and the one or a plurality of imaging markers, wherein at least one flexible elongate endoluminal instrument in the body lumen comprises at least one diagnostic sensor which receives diagnostic information of the body lumen (a diagnostic device) and is adapted to traverse longitudinally inside the body lumen, a sensor displacement measurement unit comprising a displacement sensor that measures the sensor displacement inside the body lumen, a body lumen information processor that is configured to obtain sensor displacement information from the sensor displacement measurement unit and body lumen information from the sensor, a sensor location information relative to the plurality of imaging markers, and correlates the information and optionally transmits the information to a display, wherein the diagnostic sensor is configured to perform a body lumen diagnostic scan from the inside of the body lumen by traversing longitudinally inside of the body lumen, wherein the location of the body lumen scan is referenced to the plurality of imaging markers as detected by the X-ray angiogram.

A38. The system of A37, wherein the diagnostic information of the body lumen is selected from pressure, temperature, size, oxygen level, density, or tissue morphology.

A39. The system of A37, wherein when the body lumen diagnostic sensor and the plurality of imaging markers are not on the same flexible elongate endoluminal instrument, the system further comprises: a signal transducer operating at a detectible modality and range of the body lumen diagnostic sensor affixed to the flexible elongate endoluminal instrument that comprises a plurality of imaging markers, and the locations of markers relative to the signal transducer are at a selected distance; wherein the signal transducer comprises an interface to a signal unit capable of generating and optionally receiving signals.

A40. The system of A39, wherein the location of the body lumen diagnostic sensor relative to the plurality of imaging markers is measured based on signal interactions between the signal transducer and the body lumen diagnostic sensor.

A41. The system of A39, further comprising an interface between the signal unit and body lumen information processor, wherein the location where when the signal transducer and body lumen diagnostic sensor are within a selected distance from each other, the location is measured.

A42. The system of A41, wherein the location is measured based on timing information, and optionally based on signal strength information.

A43. The system of A39, where the interface between the signal unit and body lumen detector processor is wireless.

A44. The system of A37, wherein of the plurality of imaging markers and the body lumen diagnostic sensor are mounted on the flexible elongate endoluminal instrument, and are set at a selected distance from each other when the X-ray angiogram detects the inserted flexible elongate instrument comprising the plurality of imaging markers.

A45. The system of A37, wherein the flexible elongate endoluminal instrument comprising a body lumen diagnostic sensor further comprises at least one imaging marker located at a selected distance to the body lumen diagnostic sensor.

A46. The system of A44, wherein the location of the body lumen diagnostic sensor relative to the plurality of imaging markers is measured based on the X-ray angiogram.

A47. The system of claim, wherein at least one of the plurality of imaging markers is displayed with body lumen information as a function of distance displacement in real time, or about real time during a body lumen information scan.

A48. A method for displaying the locations of imaging markers configured to be positioned on an elongated medical instrument on a diagnostic imaging display and X-ray angiogram, comprising: inserting at least one flexible elongate instrument comprising a distal end, a proximal end, and a plurality of imaging markers at the distal portion of the instrument wherein each marker dimension is of a selected width and the distances between each of the plurality of imaging markers are of a selected distance, into a body lumen, obtaining at least one X-ray angiogram of a body lumen with the inserted flexible elongate instrument such that both the body lumen and at least one of the plurality of imaging markers are detectable, and the sequence of the markers is identifiable, wherein the flexible elongate instrument further comprises at least one body lumen diagnostic sensor capable of receiving information of the body lumen (pressure, temperature, size, density, oxygen level, tissue morphology), and is configured to traverse longitudinally within the body lumen, performing a body lumen displacement scan to obtain body lumen diagnostic information, obtaining displacement information using a displacement measurement unit comprising a displacement encoding sensor, combining the displacement information with the body lumen diagnostic information obtained by the body lumen diagnostic sensor to generate position-correlated body lumen diagnostic information, measuring a location of the sensor from the body lumen scan relative to the plurality of imaging markers as detected by the X-ray angiogram, measuring a location of the body lumen diagnostic sensor relative to the plurality of imaging markers from a selected position, measuring the location of the body lumen scan relative to the plurality of imaging markers, displaying the position-correlated body lumen diagnostic information and the linear locations of the plurality of imaging markers as detected by the X-ray angiogram.

A49. The method of A48, wherein the plurality of imaging markers and the body lumen diagnostic sensor are positioned on the flexible elongate instrument, and their relative locations are determinable from the X-ray angiogram comprising the body lumen and the imaging markers.

A50. The method of A48, wherein the plurality of imaging markers are positioned on a first flexible elongate instrument, and the at least one body lumen diagnostic sensor is positioned on a second flexible elongate instrument, and the second flexible elongate instrument further comprises at least one imaging marker located at a defined distance from the body lumen diagnostic sensor such that the location of the body lumen diagnostic sensor relative to the plurality of imaging markers on the first flexible elongate instrument is measured from obtained body lumen image using the X-ray angiogram when both flexible elongate instruments are inside the body lumen.

A51. The method of A50, wherein the second flexible elongate instrument comprises a second set of a plurality of imaging markers at a selected distance from the body lumen detector, and the first set of a plurality of imaging markers is distinguishable from the second set of a plurality of imaging markers on the first flexible elongate instrument.

A52. The method of A50, wherein the plurality of imaging markers are positioned on a first flexible elongate instrument, and the at least one body lumen diagnostic sensor is positioned on a second flexible elongate instrument, and the first flexible elongate instrument further comprises a signal transducer (optionally a signal emitter, a signal receiver, or both) positioned at an defined distance from the plurality of imaging markers, and operates in a detectible modality and range of the body lumen diagnostic sensor, and the location of the body lumen diagnostic sensor is measured from the signaling between the signal transducer and the body lumen diagnostic sensor.

A53. The method of A52, wherein the signal transducer is interfaced with the body lumen diagnostic sensor to measure the distance of the body lumen diagnostic sensor in relation to the signal transducer through either signal timing and/or signal strength means, and optionally wherein the signal transducer can be in emitting or receiving mode.

A54. The method of A53, wherein the second flexible elongate instrument that comprises the body lumen detector is signally coupled to the signal transducer on the first flexible elongate instrument by using a separate transducer also mounted on the second flexible elongate instrument.

A55. The method of A48, wherein the flexible elongate instrument comprising the plurality of imaging markers is a medical guidewire.

A56. The method of A48, wherein at least one of the plurality of imaging markers is uniquely identifiable.

B1. A system for measuring the relative displacement of at least two flexible elongate instruments within a body lumen comprising: a first flexible elongate instrument comprising a proximal end, a distal end, a central axis, and one or a plurality of displacement encoding markers configured to be positioned between the proximal and distal ends, and a second flexible elongate instrument comprising a proximal end, a distal end, a central axis, and an encoding sensor which is configured to obtain a signal from the displacement encoding markers of the first flexible elongate instrument, wherein the second flexible elongate instrument is configured to traverse parallel to the central axis of the first flexible elongate instrument.

B2. The system of B1, wherein the encoding sensor comprises an interface to a signal processor that translates the obtained encoding signal to relative displacement distances between the first and second flexible elongate instruments in real-time or about real-time.

B3. The system of B 1, wherein the displacement encoding markers comprises a plurality of displacement encoding markers which are configured to be circumferentially or partially circumferentially about the first flexible elongate instrument and comprise a medium which is reflective of a signal.

B4. The system of B1, wherein the first flexible elongate instrument is configured to be positioned completely or partially inside the body lumen when used.

B5. The system of B1, wherein the medium which is reflective of a signal is selected from a metal or metal alloy, a magnet, a ceramic, a crosslinked hydrogel, or a fluoropolymer.

B6. The system of B1, wherein the first flexible elongate instrument, the second flexible elongate instrument, or both the first and second flexible elongate instruments further comprise a therapeutic and/or diagnostic device (which can include or exclude a diagnostic or treatment device) which is configured to be positioned at the distal portion of said elongate instrument.

B7. The system of B6, wherein the location of the displacement encoding markers and/or the encoding sensor is known when at least one flexible elongate instrument comprises a therapeutic and/or diagnostic device.

B8. The system of B1, wherein the therapeutic and/or diagnostic device is a diagnostic device which obtains body lumen information.

B9. The system of B8, wherein the diagnostic device is in electronic or optical communication with the signal processor.

B10. The system of B9, wherein the signal processor calculates from the obtained displacement information, body lumen information per displacement distance.

B11. The system of B10, wherein the body lumen information per displacement distance is electronically transmitted to a display.

B12. The system of B11, wherein the display is a component of a diagnostic system.

B13. The system of B12, wherein the diagnostic system is IVUS.

B14. The system of B10, wherein the body lumen information is selected from tissue density, temperature, pressure, flow rate, impedance, or conductivity.

B15. A system for measuring the location of a therapeutic and/or diagnostic device when within a body lumen in reference to selected positions of said body lumen, comprising: a first flexible elongate instrument comprising one or a plurality of displacement encoding markers positioned on the first flexible elongate instrument and one or a plurality of radiopaque imaging markers positioned on the first flexible elongate instrument, and a second flexible elongate instrument comprising a proximal end, a distal end, and an encoding sensor, wherein the encoding sensor and the displacement encoding markers on the first flexible elongate instrument, forms a first engagement position when the encoding sensor begins to detect the displacement encoding markers, at least one X-ray angiogram image of a body lumen with the flexible elongate instrument inserted completely or partially therein wherein the image comprises one or a plurality of radiopaque imaging markers on the flexible elongate instrument, such that both the body lumen and the plurality of radiopaque imaging markers are identifiable, and at least one of the radiopaque imaging markers is individually identifiable, wherein the obtained X-ray angiogram image identifies a location of the plurality of radiopaque imaging markers in the body lumen, and wherein the second flexible elongate instrument is configured to traverse parallel to the longitudinal axis of the first flexible elongate instrument.

B16. The system of B15, wherein the location of the function device relative to the location of the plurality of radiopaque imaging markers as obtained by the X-ray angiogram image is (optionally, continuously) measured.

B17. The system of B15, further comprising a plurality of radiopaque imaging markers configured to be positioned on the first or the second flexible elongate instrument, such that the position of the plurality of radiopaque imaging markers to either the encoded region, or to the encoding sensor on the selected flexible elongate instrument is known.

B18. The system of B16, wherein the first or second flexible elongate instrument is a therapeutic and/or diagnostic device, and the position of the therapeutic and/or diagnostic device relative to the encoded region or the encoding sensor on the flexible elongate instrument is at a selected distance, and optionally further defines a start location, which is the location of the therapeutic and/or diagnostic device relative to the plurality of radiopaque imaging markers at the first engagement position.

B19. The system of B18, further comprising a signal processor which is configured to obtain a signal from the encoding sensor, convert the encoding signal to displacement information location, and calculate locations.

B20. The system of B19, wherein the signal processor displays the locations and diagnostic information obtained from the therapeutic and/or diagnostic device relative to the location of the plurality of radiopaque imaging markers obtained from the X-ray angiogram image.

B21. The system of B18, wherein the start location is obtained by the signal processor when the encoding sensor first begins to detect the displacement encoding markers.

B22. The system of B21, wherein the signal processor continuously or intermittently obtains the data from the encoding sensor and associates the location of the therapeutic and/or diagnostic device relative to the plurality of radiopaque imaging markers.

B23. The system of B15, further comprising a display.

B24. The system of B15, wherein the therapeutic and/or diagnostic device provides diagnostic information at each tested location, the diagnostic sensor further comprises an interface to a signal processor, and the signal processor displays the diagnostic body lumen information relative to the location of the plurality of radiopaque imaging markers as presented in the X-ray angiogram image.

B25. The system of B15, wherein the locations of the therapeutic and/or diagnostic device are presented to the display in such a manner that the locations of the therapeutic and/or diagnostic device relative to the location of the plurality of radiopaque imaging markers as obtained in the X-ray angiogram image on a simulated line are depicted.

B26. The system of any of B15-B25, wherein the presentation of the locations of the therapeutic and/or diagnostic device within the body lumen are presented to the display in real time or about real-time.

B27. The system of B15, wherein when performing displacement measurements at a plurality of different times (and optionally using different therapeutic and/or diagnostic devices) the locations of the therapeutic and/or diagnostic devices and associated diagnostic information provided by the diagnostic device are provided to the display when the locations from the measurements at a plurality of times are measured relative to the location of the plurality of radiopaque imaging markers as obtained from the X-ray angiogram image.

B28. The system of B15, wherein the signal emitted and/or obtained by the displacement encoding markers and the encoding sensor is selected from optical, electro-magnetic, capacitive, or acoustic.

B29. A computer-implemented method for measuring the relative displacement of a second flexible elongate instrument relative to a first flexible elongate instrument when both the first and second flexible elongate instruments are positioned to be wholly or partially within a body lumen comprising: receiving a plurality of encoding signals from an encoding sensor which is a component of a second flexible elongate instrument comprising a proximal end, a distal end, and an encoding sensor, which is inserted into a body lumen, wherein the encoding signals are reflective of one or a plurality of encoding markers which are a component of a first flexible elongate instrument inserted into a body lumen, transmitting the plurality of encoding signals from the encoding sensor to a signal processor which converts the obtained encoding signals to one or a plurality of displacement values of the relative displacement difference between the first and second flexible elongate instruments to calculate the relative displacements, and transmitting the calculated relative displacements through an interface to a display, wherein the first or the second flexible elongate instrument or both, further comprise at least one therapeutic and/or diagnostic device.

B30. The method of B29, wherein the at least one therapeutic and/or diagnostic device is selected from a body lumen diagnostic sensor capable of obtaining diagnostic information about the body lumen and is further interfaced to the signal processor, and generates body lumen information at each relative displacement.

B31. A system comprising at least one non-transitory machine-readable medium storing instructions which, when executed by a programmable processor, cause the programmable processor to perform operations comprising the methods of any of B29-30.

B32. A computer-implemented method for measuring the position of a first flexible elongate instrument within a body lumen comprising: obtaining encoding information obtained from having the following steps performed: (i) inserting into a body lumen a first flexible elongate instrument which either comprises a plurality of displacement encoding markers or comprises an encoding sensor, (ii) inserting into a body lumen a second flexible elongate instrument configured to be used in conjunction with the first flexible elongate instrument and wherein the second flexible elongate instrument comprises an encoding sensor when the first flexible elongate instrument comprises a plurality of displacement encoding markers or the second flexible elongate instrument comprises a plurality of displacement encoding markers when the first flexible elongate instrument comprises an encoding sensor, (iii) obtaining an encoding signal from the displacement encoding markers as detected by the encoding sensor to generate encoding information, obtaining at least one X-ray angiogram image of a body lumen with the flexible elongate instrument comprising a plurality of radiopaque imaging markers placed partially or entirely inside the body lumen such that both the body lumen and the plurality of radiopaque imaging markers are identifiable, and at least one of the plurality of radiopaque imaging markers is individually identifiable, wherein the obtained angiographic image defines a location of the plurality of radiopaque imaging markers in the body lumen, identifying a first location of the displacement encoding markers relative to the location of the plurality of radiopaque imaging markers as obtained by the X-ray angiogram image, transmitting the encoding information to a signal processor, and processing the encoding information by the signal processor to translate the encoding information into a spatial displacement between the first and second flexible elongate instrument to identify the position of the first flexible elongate instrument, wherein the first flexible elongate instrument or the second flexible elongate instrument further comprises a plurality of radiopaque imaging markers and the position of the radiopaque imaging markers to the displacement sensor or plurality of displacement encoding markers on the respective flexible elongate instrument is of a selected distance.

B33. The method of B31, wherein the location of the function device is continuously measurable.

B34. The method of B31, wherein the step of obtaining at least one X-ray angiogram image of a body lumen with the flexible elongate instrument comprising a plurality of radiopaque imaging markers placed partially or entirely inside the body lumen such that both the body lumen and the plurality of radiopaque imaging markers are identifiable is performed before first engagement of the encoding sensor and the encoding but the flexible elongate instrument with the plurality of radiopaque imaging markers has not moved from its imaged position when first engagement occurred.

B35. The method of B31, wherein the start location is obtained by the signal processor.

B36. The method of B31, wherein the location of the flexible elongate instrument comprising the plurality of displacement encoding markers relative to the plurality of radiopaque imaging markers is continuously or intermittently measurable.

B37. The method of B35, wherein the periodicity of the intermittent measurements is once per 0.1 sec, once per 1 sec, once per 10 sec, once per minute, once per 5 minutes, once per 10 minutes, once per 20 minutes, once per 30 minutes, once per 40 minutes, once per 50 minutes, or once per hour.

B38. The method of B31, wherein the first flexible elongate instrument or second flexible elongate instrument further comprises a therapeutic and/or diagnostic device.

B39. The method of B37, further comprising displaying the locations and obtained diagnostic information from the therapeutic and/or diagnostic device relative to the location of the plurality of radiopaque imaging markers as obtained in the X-ray angiogram image.

B40. The method of B37, wherein a therapeutic and/or diagnostic device is located either on the first or the second flexible elongate instrument, such that the position of the therapeutic and/or diagnostic device to either the encoded region or the encoding sensor is known on the flexible elongate instrument, and further defines a start location, which is the location of the therapeutic and/or diagnostic device relative to the plurality of radiopaque imaging markers when the encoding sensor begins to obtain signals from the displacement encoding markers.

B41. The method of B37, wherein the clinician is alerted when the first engagement occurs.

B42. The method of B41, wherein the alert is selected from: audio (which can include a sound) or visual (which can include a light or a message communicated to a display) or physical (which can include or exclude haptic feedback signals).

B43. A system comprising at least one non-transitory machine-readable medium storing instructions which, when executed by a programmable processor, cause the programmable processor to perform operations comprising the methods of any of B32-42.

B44. A computer-implemented method for measuring the position of a therapeutic and/or diagnostic device within a body lumen comprising: obtaining information from an inserted first flexible elongate instrument which either comprises a displacement encoding markers at a location typically positioned inside the body lumen during use, or comprises an encoding sensor, obtaining information from an inserted second flexible elongate instrument configured to be used in conjunction with the first flexible elongate instrument, wherein the first flexible elongate instrument or the second flexible elongate instrument comprises an encoding sensor which obtains an encoding signal, or one or a plurality of displacement encoding markers providing encoding information to the encoding sensor, (depends on the design of the first flexible elongate instrument) wherein in conjunction with the first flexible elongate instrument, comprises a first engagement position, such that the encoding sensor first engage with the encoded region in normal clinical use, wherein a plurality of radiopaque imaging markers are located either the first or the second flexible elongate instrument such that the position of the plurality of radiopaque imaging markers to either the encoded region, or the encoding sensor is known on the flexible elongate instrument, wherein a therapeutic and/or diagnostic device is located either on the first or the second flexible elongate instrument, such that the position of the therapeutic and/or diagnostic device to either the encoded region or the encoding sensor is known on the flexible elongate instrument, and further defines a start location, which is the location of the therapeutic and/or diagnostic device relative to the plurality of radiopaque imaging markers at the first engagement position, interfacing the encoding sensor to a signal processor capable of translating the encoding signal to displacement between the first and second flexible elongate instrument, and the signal processor has interface to receive other inputs, and is interfaced to a display, wherein the start location is obtained by the signal processor, wherein upon first engagement of the two flexible elongate instruments, the location of the therapeutic and/or diagnostic device relative to the plurality of radiopaque imaging markers is continuously measurable, obtaining at least one X-ray angiogram image of a body lumen with the flexible elongate instrument with a plurality of radiopaque imaging markers placed inside the body lumen such that both the body lumen and the plurality of radiopaque imaging markers are identifiable, and at least one of the plurality of radiopaque imaging markers is individually identifiable, wherein the obtained angiographic image defines a location of the plurality of radiopaque imaging markers in the body lumen, measuring a first location of the therapeutic and/or diagnostic device relative to the location of the plurality of radiopaque imaging markers as obtained by the X-ray angiogram image is measured, either (i) the angiographic image is obtained after first engagement of the encoding sensor and the encoding and therefore the location of the function device is already continuously measurable, or (ii) the angiographic image is obtained before first engagement of the encoding sensor and the encoding but the flexible elongate instrument with the plurality of radiopaque imaging markers has not moved from its imaged position when first engagement occurred, optionally, continuously measuring the location of the function device relative to the location of the plurality of radiopaque imaging markers as obtained by the X-ray angiogram image, optionally, displaying the locations and associated information of the therapeutic and/or diagnostic device relative to the location of the plurality of radiopaque imaging markers as obtained in the X-ray angiogram image.

B45. The method of B40, wherein the function device is selected from a body lumen diagnostic sensor generating diagnostic body lumen information at each measured location, and the diagnostic sensor further comprises an interface to the signal processor, and the signal processor displays the diagnostic body lumen information relative to the location of the plurality of radiopaque imaging markers as obtained in the X-ray angiogram image.

B46. The method of B41, display the locations of the therapeutic and/or diagnostic device relative to the linear position of the plurality of radiopaque imaging markers as obtained in the X-ray angiogram image on a simulated line.

B47. The method of B40, wherein the information is displayed in real time or about real-time as they are being received and calculated.

B48. The method of B40, when performing displacement measurements from different time point, and optionally using different therapeutic and/or diagnostic devices, the locations of therapeutic and/or diagnostic devices and associated information are overlapping displayed (on a single image) when the locations from the different measurements are measured relative to the location of the plurality of radiopaque imaging markers as obtained by the same X-ray angiogram image.

B49. A system comprising at least one non-transitory machine-readable medium storing instructions which, when executed by a programmable processor, cause the programmable processor to perform operations comprising the methods of any of B42-B46.

B50. A system for identifying in real-time or about the location of a therapeutic and/or diagnostic device when within a body lumen comprising: a first flexible elongate instrument comprising a proximal end, a distal end, a central axis, and one or a plurality of displacement encoding markers, a second flexible elongate instrument comprising a proximal end, a distal end, and an encoding sensor, wherein the second flexible elongate instrument is configured to traverse along the first flexible elongate instrument substantially parallel to the central axis of the first flexible elongate instrument, wherein a first engagement position is defined when the displacement encoding markers on the first flexible elongate instrument are first detected by the encoding sensor, a plurality of radiopaque imaging markers located either on the first or the second flexible elongate instrument, such that the linear position of the plurality of radiopaque imaging markers to either the encoded region, or the encoding sensor is known on the selected flexible elongate instrument, a therapeutic and/or diagnostic device located on the flexible elongate instrument that does not comprise the plurality of radiopaque imaging markers, such that the position of the therapeutic and/or diagnostic device to either the encoded region or the encoding sensor is known on the flexible elongate instrument, wherein when the first and second flexible elongate instrument are at the first engagement position, the location of the therapeutic and/or diagnostic device to the plurality of radiopaque imaging markers is known, a signal processor which is configured to obtain a signal from the encoding sensor and optionally from the therapeutic and/or diagnostic device, converts the encoding signal to a relative displacement distance, optionally performs location calculations, and optionally further comprises an interface to a display, wherein the relative distance between the therapeutic and/or diagnostic device and the plurality of radiopaque imaging markers at the first engagement position is obtained by the signal processor, an X-ray imaging system which is configured to obtain and display one or a plurality of images of the plurality of radiopaque imaging markers in the body lumen, a display, wherein the signal processor transmits to the display a simulated representation of the therapeutic and/or diagnostic device relative to the positions of the plurality of radiopaque imaging markers in real-time or about real-time.

B51. The system of B50, wherein after the encoding sensor first engages with the encoded region, the locations of the therapeutic and/or diagnostic device on one flexible elongate instrument relative to the plurality of radiopaque imaging markers on the other flexible elongate instrument are continuously measurable.

B52. The system of B50, wherein the update rate for the X-ray imaging is lower than the update rate for the simulated representation of the function device relative to the positions of the plurality of radiopaque imaging markers display.

B53. The system of B50, wherein the X-ray imaging system is further configured to repeatably update the one or plurality of images in real-time or about real-time as the first flexible elongate instrument moves relative to the second flexible elongate instrument.

B54. The system of B53, wherein the repeat rate is selected from once per 0.1 sec, 1 sec, 2 sec, 3 sec, 4 sec, 5 sec, 6 sec, 7 sec, 8 sec, 9 sec, 10 sec, 20 sec, 30 sec, 40 sec, 50 sec, 60 sec, 2 min, 3 min, 4 min, 5 min, 6 min, 7 min, 8 min, 9 min, 10 min, or any rate between the aforementioned rates.

B55. The system of B50, wherein upon the first engagement, a signal is sent to the clinician which is selected from an audio, visual, or physical signal.

B56. A computer-implemented method for measuring the position of therapeutic and/or diagnostic device within a body lumen comprising: a. obtaining displacement encoding information from a first flexible elongate instrument and a second flexible elongate instrument obtained from a process comprising: (i) inserting a first flexible elongate instrument comprising a proximal end, a distal end, a central axis, and one or a plurality of displacement encoding markers into a body lumen, (ii) inserting a second flexible elongate instrument comprising a proximal end, a distal end, and an encoding sensor, wherein the second flexible elongate instrument is configured to traverse along the first flexible elongate instrument substantially parallel to the central axis of the first flexible elongate instrument into said body lumen, (iii) forming a first engagement position when the displacement encoding markers on the first flexible elongate instrument are first detected by the encoding sensor, (iv) detecting the displacement encoding markers with the encoding sensor to generate displacement encoding information, wherein a plurality of radiopaque imaging markers is located either on the first or the second flexible elongate instrument, such that the linear position of the plurality of radiopaque imaging markers to either the encoded region, or the encoding sensor is known on the selected flexible elongate instrument, wherein a therapeutic and/or diagnostic device is located on the flexible elongate instrument that does not comprise the plurality of radiopaque imaging markers, such that the position of the therapeutic and/or diagnostic device to either the encoded region or the encoding sensor is known on the flexible elongate instrument, and wherein when the first and second flexible elongate instrument are at the first engagement position, the location of the therapeutic and/or diagnostic device to the plurality of radiopaque imaging markers are known, b. obtaining one or a plurality of X-ray images of the plurality of radiopaque imaging markers in the body lumen, c. translating the displacement encoding information to the known position of the radiopaque imaging markers to measure the position of the flexible elongate instrument comprising the therapeutic and/or diagnostic device to the position of the radiopaque imaging markers to generate the measured position of the therapeutic and/or diagnostic instrument in the body lumen relative to the radiopaque imaging markers, wherein a plurality of radiopaque imaging markers are located either the first or the second flexible elongate instrument such that the position of the plurality of radiopaque imaging markers to either the encoded region, or the encoding sensor is known on the flexible elongate instrument, wherein a therapeutic and/or diagnostic device is located on the elongate instrument that do not have the plurality of radiopaque imaging markers, such that the position of the therapeutic and/or diagnostic device to either the encoded region or the encoding sensor is known on the flexible elongate instrument.

B57. The method of B56, further comprising displaying a simulated representation of the therapeutic and/or diagnostic device relative to the positions of the plurality of radiopaque imaging markers in real-time or about real-time.

B58. The method of B56, wherein after the encoding sensor first engages with the encoded region, the locations of the therapeutic and/or diagnostic device on one flexible elongate instrument relative to the plurality of radiopaque imaging markers on the other flexible elongate instrument are continuously measured.

B59. The computer-implemented method of B58, wherein the update rate for the X-ray image is lower than the update rate for the simulated representation of the function device relative to the positions of the plurality of radiopaque imaging markers display.

B60. A system comprising at least one non-transitory machine-readable medium storing instructions which, when executed by a programmable processor, cause the programmable processor to perform operations comprising a method of any of B50-B59.

C1. A co-location system comprising: at least one first flexible elongate instrument comprising a proximal end, a distal end, a device position acquisition unit, which is shaped and adapted for insertion into a body lumen and further comprises a plurality of imaging markers circumferentially and/or partially circumferential positioned around each of the at least one flexible elongate instrument, at least one second flexible elongate instrument wherein the second flexible elongate instrument is a therapeutic and/or diagnostic device, a sensor which detects the relative movement of the flexible elongate instruments, a display and/or an interface to a display, which optionally comprises an interface to an input/output device, an interface to an external body imaging device which obtains one or a plurality of body images and provides said body images to a calculation unit, an interface to a computer network, and a calculation unit which is configured to generate one or a plurality of 2D and/or 3D models of the at least one flexible elongate instrument positions within a body lumen, calculate co-location information of the second flexible elongate instrument with said models, and is connected to the interface of a display and optionally is connected to an input/output device, wherein at least the first flexible elongate instrument and/or second flexible elongate instrument comprises a plurality of displacement encoding markers, wherein the co-location information comprises the positional information of the therapeutic and/or diagnostic device position within the body lumen, wherein the calculation unit optionally sends to the interface to a display electronic data which displays an image of data obtained from the at least one therapeutic and/or diagnostic device at one or multiple locations along the first flexible elongate instrument to the display, and wherein the at least one therapeutic and/or diagnostic device provides positional information to the calculation unit.

C2. The co-location system of C1, wherein the first flexible elongate instrument is electronically or wirelessly connected to the calculation unit.

C3. The co-location system of C2, wherein the therapeutic and/or diagnostic device is electronically or wirelessly connected to the calculation unit.

C4. The co-location system of C1, wherein the sensor is positioned outside the body of the patient.

C5. The co-location system of C3, wherein the sensor is configured to be within a robotic arm.

C6. The co-location system of C3, wherein the calculation unit constructs the 2D and/or 3D models for the position of the first flexible elongate instrument within the body lumen at a separate time from acquiring body images.

C7. The co-location system of C1, wherein the first flexible elongate instrument is configured to further comprise the sensor.

C8. The co-location system of C1, wherein the therapeutic and/or diagnostic device is further configured to comprise displacement encoding markers.

C9. The co-location system of C1, wherein the first flexible elongate instrument is selected from a guidewire or a catheter.

C10. The co-location system of C1, wherein the plurality of imaging markers are each independently of a selected distance from each other.

C11. The co-location system of C1, wherein the plurality of imaging markers are each independently of a selected dimension.

C12. The co-location system of C1, wherein the therapeutic and/or diagnostic device comprises a central axis that is positioned parallel to, or sharing about the same center of axis as the first flexible elongate instrument and is configured to travel parallel to the axis of the first flexible elongate instrument.

C13. The co-location system of C12, wherein the calculation unit detects movement or the distance of the therapeutic and/or diagnostic device has traversed along the first flexible elongate instrument relative to the fixed position of the first flexible elongate instrument by comparing a first signal transmitted from the sensor upon detection of the plurality of displacement encoding markers and/or the signal from the said therapeutic and/or diagnostic device from a second signal transmitted from said sensor and/or therapeutic and/or diagnostic device.

C14. The co-location system of C1, wherein the sensor is selected from an optical sensor, an electrical sensor, or a sonographic sensor.

C15. The co-location system of C1, wherein the therapeutic and/or diagnostic device is IVUS.

C16. The co-location system of C1, wherein the interfaces to the display, X-ray angiogram imaging device, and computer network are bi-directional.

C17. The co-location system of C16, wherein the interfaces are selected from wired (electronically connected via solid-line communication) or wireless (electronically connected via communication via wavelength transmitters and receivers).

C18. The co-location system of C1, wherein the system is configured to be an independent instrument.

C19. The co-location system of C1, wherein the system is configured to be a component of a body imaging system, or a component of a therapeutic and/or diagnostic system.

C20. The co-location system of C1, wherein the connection to the interface of a display is selected from an electronic connection or a wireless connection.

C21. The co-location system of C1, wherein the connection to the input/output device is selected from an electronic connection or a wireless connection.

C22. The co-location system of C1, wherein the calculation unit is configured to:

receive at least one external body image of a body lumen with the first flexible elongate instrument inserted in the lumen, and/or body lumen location information; generate one or a plurality of 2D and/or 3D models of a selected section of the first flexible elongate instrument from its external body images and the plurality of imaging markers located on the 2D/3D model of the instrument section; calculate the body lumen position co-location with the external body lumen image and/or data, and/or corresponding diagnostic and/or therapeutic device data, and/or data from an input/out device with said one or a plurality of 2D and/or 3D models; generate a simulated representation of the dimension and position of the therapeutic and/or diagnostic device located within the body lumen as a 2D and/or 3D illustration to form a simulated device image; generate one or a plurality of images that overlay the one or a plurality of 2D and/or 3D models and the simulated device image with the one or plurality of body images, and/or with the corresponding diagnostic and/or therapeutic device data, and/or an input/out device; display the therapeutic and/or diagnostic device 2D and/or 3D illustration with the one or a plurality of 2D and/or 3D models; optionally display the said therapeutic and/or diagnostic device 2D and/or 3D illustration and the position information on the external body image, and/or on the corresponding diagnostic and/or therapeutic device data, and/or an input/out device; optionally, obtain diagnostic and/or therapeutic information from the therapeutic and/or diagnostic device; optionally, display the diagnostic and/or therapeutic information obtained from the therapeutic and/or diagnostic device and/or body lumen location at one or a plurality of selected locations on the first flexible elongate instrument; optionally, enable at least one interactive display among diagnostic and/or therapeutic devices/systems, control device/system and displays; optionally, store the position information, co-location image and data locally, optionally, transmit the position information and co-location data to a separate local system and/or local computer network and/or outside computer network.

C23. The co-location system of C22, wherein the calculation of the co-location configuration element (c) is performed in real time or about real-time with obtaining the therapeutic and/or diagnostic device positions relative to the first flexible elongate instrument from the sensor.

C24. The co-location system of C22, wherein the calculation of the co-location configuration element (c) is performed separately from obtaining the therapeutic and/or diagnostic device positions on the flexible elongate instrument relative to the first flexible elongate instrument from the sensor.

C25. The co-location system of C22, wherein the step (i) display the diagnostic and/or therapeutic information obtained from the therapeutic and/or diagnostic device at one or a plurality of selected locations on the first elongate instrument is performed at the same time or about the same time as when step (a) obtain device positions on the flexible elongate instrument from a sensor, is performed.

C26. The co-location system of \C22, wherein the step (i) display the diagnostic and/or therapeutic information obtained from the therapeutic and/or diagnostic device at one or a plurality of selected locations on the first elongate instrument is performed at a separate time as when step (a) obtain device positions on the flexible elongate instrument from the sensor, is performed.

C27. The co-location system of C22, wherein the step (j) enable at least one interactive display among diagnostic and/or therapeutic devices/systems, control device/system and displays, comprises obtaining position sensing data from an input/output device.

C28. The co-location system of any of C1-27, wherein the external body imaging system is X-ray angiography, and the external body image is an X-ray angiogram.

C29. A flexible elongate instrument comprising a proximal end, a distal end, and a sensor, which is shaped and adapted for insertion into a body lumen and further comprises a plurality of imaging markers circumferentially positions circumferentially around the flexible elongate instrument.

C30. The flexible elongate instrument of C29, wherein the plurality of imaging markers are independently of a selected distance from each other.

C31. The flexible elongate instrument of C29, wherein the plurality of imaging markers are independently of a selected dimension, wherein the dimension of the imaging markers are of a selected width.

C32. The flexible elongate instrument of C29, wherein the number of imaging markers ranges from 2 to 500.

C33. The flexible elongate instrument of any of C29-32, wherein the imaging markers are radiopaque.

C34. A method for measuring the position of a portion or all of a flexible elongate instrument within a body lumen, the method comprising: obtain an image of a first image of part or all of the body lumen of a patient, wherein the body lumen comprises an inserted flexible elongate instrument comprising a plurality of imaging markers; delineating the outline of the part or all of the body lumen; associating the position of the flexible elongate instrument within the body lumen; developing a 2-D and/or 3-D model of the part or all of the body lumen; and generating geometry of the inserted flexible elongate instrument in the body lumen such that the position of a portion or all of the flexible elongate instrument within the body lumen is measured.

C35. The method of C34, wherein the associating the position of the flexible elongate instrument within the body lumen is performed by receiving electronic information from the flexible elongate instrument as to its relative position within the body lumen.

C36. The method of C34, wherein developing a 2-D and/or 3-D model of the body lumen comprises identifying boundary points on the body lumen and fitting the 2-D and/or 3-D model of the body lumen to the boundary points.

C37. A method for constructing one or a plurality of 2-dimensional models of a flexible elongate instrument which has been inserted into a body lumen of a patient, comprising:

obtaining positional data electronic information from the flexible elongate instrument inserted into a body lumen of a patient, wherein the flexible elongate instrument comprises a proximal end, a distal end, and a plurality of imaging markers positioned circumferentially about the flexible elongate instrument, obtaining one or a plurality of images of the plurality of imaging markers within the body lumen, generating a 2-dimensional model depicting dimension information of the flexible elongate instrument when inside the body lumen from the at least one image and the positional data electronic information obtained from the flexible elongate instrument, wherein the dimension information is calculated from the known spacing and dimensions of the plurality of imaging markers.

C38. A method for constructing a 3-dimensional model of a flexible elongate instrument which has been inserted into a body lumen, comprising: obtaining positional data electronic information from the flexible elongate instrument inserted into a body lumen of a patient, wherein the flexible elongate instrument comprises a proximal end, a distal end, and a plurality of imaging markers positioned circumferentially about the flexible elongate instrument, obtaining at least two separate images from at least two orientations of the plurality of radiopaque markers within the body lumen, generating a 3-dimensional model of the flexible elongate instrument in the body lumen from the images obtained and the positional data electronic information obtained from the flexible elongate instrument wherein the dimension information is calculated from the known spacing and dimensions of the plurality of imaging markers.

C39. The method of any of A37 or A38, wherein the one or a plurality of images are X-ray images, preferably X-ray angiograms.

C40. The method of C39, further comprising: recording at least one body lumen image with the first flexible elongate instrument positioned within the body lumen, with a plurality of imaging markers positioned partially or wholly inside the body lumen at the same orientation as the model, aligning the markers from the 2-dimensional model with the imaging markers on the at least one recorded image as a correlated unit, superimposing on a display the body-lumen image with the model of the first flexible elongate instrument from the aligned radiopaque markers on a display.

C41. The method of C39, further comprising: storing the at least one body lumen image with the first flexible elongate instrument with a plurality of imaging markers inside the body lumen from a selected orientation in a physical medium, aligning the imaging markers from the 3-dimensional model with the markers on the recorded image with that orientation as correlated unit, superimposing the body lumen image from the new orientation with the model of the first flexible elongate instrument from the aligned imaging markers on a display.

C42. The method of any of C40 or C41, further comprising: storing to a physical medium a second body-lumen image obtained from the plurality of imaging markers within the body lumen, aligning the endo-lumen positions of the two stored body lumen images, identifying differences in one or a plurality of selected imaging marker positions between the two stored body lumen images, measuring the differences in one or a plurality of selected imaging marker positions between the two stored body lumen images to obtain a self-correction coefficient, and optionally, applying the self-correction coefficient to a subsequent body-lumen image obtained from the plurality of imaging markers within the body lumen.

C43. The method of C39, further comprising: generating a 2-dimensional model of a body lumen with a first flexible elongate instrument inside the body lumen with dimensions by a method comprising: obtaining at least one image of the body lumen comprising a flexible elongate instrument partially or completely inside the body lumen which comprises a plurality of imaging markers each independently having a selected distance and width, and generating a 2-dimensional model of the body lumen with the first flexible elongate instrument inside the body lumen, where the positions of the plurality of imaging markers relative to the body lumen model are measured.

C44. The method of C39, further comprising: generating a 3-dimensional (3-D) model of a body lumen with a first flexible elongate instrument inside the body lumen with dimensions by a method comprising: obtaining at least two images of the body lumen comprising a flexible elongate instrument partially or completely inside the body lumen which comprises a plurality of imaging markers each independently having a selected distance and width from at least two orientations, and generating a 3-dimensional model of the body lumen with the first flexible elongate instrument inside the body lumen, where the position of the plurality of imaging markers relative to the body lumen model are measured.

C45. The method of any of C43 or C44, wherein the calculation of the dimension information is calculated from the known spacing and dimensions of the plurality of imaging markers is generated from the distance encoding built into at least one flexible elongate instrument.

C46. The method of any of C43 or C44, wherein the displayed position and the associated dimension information of the said another device along the first flexible elongate instrument is superimposed with the 2D and/or 3D model of the first flexible elongate instrument.

C47. The method of any of C34-46, wherein the imaging markers are radiopaque and the body imaging system is X-ray angiography.

C48. A body lumen signal correlation processing system comprising: one or a plurality of flexible elongate instruments wherein each flexible elongate instrument comprises a plurality of imaging markers, wherein the imaging markers are visible by an external body imager, and the imaging markers comprises a length and distance between each marker which are of a selected dimension, and at least one imaging marker is uniquely identifiable; an external body imager configured to obtain one or a plurality of body lumen images from one or a plurality of orientations, with the flexible elongate instrument inserted in the body lumen, wherein the body lumen image comprises an image of the body lumen and one or a plurality of imaging markers, an interface to a calculation unit which is configured to transmit body lumen location information relative to the plurality of imaging markers; a processor capable of receiving imaging information from the external body imager and body lumen location information in reference to the plurality of imaging markers; and a display or an interface to a display, and optionally an interface to an input/output device.

C49. The body lumen signal correlation processing system of C48, wherein the processor is configured to receive at least one external body image of a body lumen with the flexible elongate instrument inserted in the body lumen, and both the body lumen outline and the plurality of imaging markers are detected, and the at least one individually identifiable marker is in the field of image.

C50. The body lumen signal correlation processing system of C48, wherein the processor is configured to generate a 2D/3D model of a selected section of the flexible elongate instrument with the plurality of imaging markers located on the 2D/3D model of the instrument, wherein the model is generated by a relationship of the received at least one image, and the linear distance scale along the flexible elongate instrument (measured based on the known marker lengths and known gap between each imaging marker dimension).

C51. The body lumen signal correlation processing system of C48, wherein the processor is configured to generate a 2D/3D model of the body lumen segment with the plurality of imaging markers located in the 2D/3D model of the body lumen segment, and measure the linear distance scale along the central axis of the body lumen.

C52. The body lumen signal correlation processing system of C51, wherein the processor is configured to measure the location of one or a plurality of body lumen on the 2D/3D model based on a selected relationship between the said body lumen location information in reference to the plurality of imaging markers.

C53. The body lumen signal correlation processing system of C48, wherein the display is configured to display the body lumen location with the constructed model.

C54. The body lumen signal correlation processing system of C48, wherein the processor is configured to overlay the body lumen location with an external body image of the body lumen by aligning the plurality of imaging markers between the constructed model and the external body image.

C55. The body lumen signal correlation processing system of C48, wherein the processor is configured to display the body lumen location in real-time and near real-time.

C56. The body lumen signal correlation processing system of C48, wherein when the body lumen location information received by the processor is the location of a body lumen diagnostic sensor, and the processor is further configured to receive information from the diagnostic sensor, and correlates the diagnostic sensor information with the sensor location information, and the diagnostic sensor information can optionally be selectively be displayed at a selected location.

C57. The body lumen signal correlation processing system of C48, wherein the model of the flexible elongate instrument is generated with a recaptured external body image, and the location of the device on the flexible elongate instrument model is also generated and overlayed on the display with the recaptured external body image.

C58. The body lumen signal correlation processing system of C57, wherein the external body image is an X-ray angiogram, and the model of the flexible elongate instrument is generated when the X-ray instrument is not emitting X-rays.

C59. The body lumen signal correlation processing system of C49, wherein the body lumen location information received by the processor is the location of a device with defined geometric dimension, and the processor generates a simulated representation of the therapeutic and/or diagnostic device and displays the representation on the elongate instrument and/or the body lumen model, or optionally overlaid and displayed the representation and position information on the external body image, or optionally the corresponding diagnostic and/or therapeutic device data.

C60. The body lumen signal correlation processing system of C48, wherein the processor is configured to interface with at least one component within the said system, optionally in a bidirectional manner.

C61. The body lumen signal correlation processing system of C48, wherein the processor comprises a calculation component, a storage component, and an input/output interface.

C62. The body lumen signal correlation processing system of C48, wherein the interfaces and the connections with the system are selected from wired (electronically connected via solid-line communication) or wireless (electronically connected via communication via wavelength transmitters and receivers), optionally in a bidirectional manner.

C63. The body lumen signal correlation processing system of C62, wherein the interfaces are selected from wired (electronically connected via solid-line communication) or wireless (electronically connected via communication via wavelength transmitters and receivers), and optionally bidirectional.

C64. The body lumen signal correlation processing system of C48, wherein the one and/or multiple body lumen location information is displayed about simultaneously on the at least one model and/or on the external body image, or optionally the diagnostic and/or therapeutic system data, or optionally on at least one display.

C65. The body lumen signal correlation processing system of C48, wherein the processor is configured to store processed information locally, and optionally store processed information in an external computer network via interface.

C66. The body lumen signal correlation processing system of C48, wherein the processor is configured to be positioned in a separate housing than the other components of the system.

C67. The body lumen signal correlation processing system of C48, wherein the processor is configured to be a component of an external body imaging system, and/or a component of a diagnostic and/or therapeutic device and/or system.

C68. A method of displaying a body lumen location on a body lumen image, comprising: obtaining at least one body image of a body lumen comprising a lumen-inserted flexible elongate instrument comprising a plurality of imaging markers into a body lumen, wherein the markers are visible by an external body imager, and each imaging marker dimension and spacing between each imaging markers are known, and at least one imaging marker is individually identifiable, from at least one orientation, wherein both the body lumen outline and the plurality of imaging markers are detectable, and the at least one individually identifiable imaging marker is in the field of image, constructing a 2D or 3D model of the flexible elongate instrument within the body lumen, and optionally the lumen, with the positions of the imaging markers, and displaying the linear distance scale along the central axis of the flexible elongate instrument within the body lumen, receiving body lumen locations of one or a plurality of an inserted diagnostic and/or therapeutic device inserted into the body lumen and having a central axis which traverses the central axis of the flexible elongate instrument body lumen locations, wherein the positions are calculated relative to the positions of the plurality of imaging markers, and calculating the received body lumen locations on the model, and displaying the body lumen locations of the inserted diagnostic and/or therapeutic device on the external body image by overlaying the model with the external body image using the plurality of imaging markers for alignment.

C69. The method of C68, wherein the display of location is in real-time or near real-time.

C70. The method of C68, wherein the location is from a diagnostic sensor, and the diagnostic sensor information is correlated with the body lumen location information, and the diagnostic sensor information is selectively displayed at selected positions on the image of the body lumen.

C71. The method of C68, wherein the flexible elongate instrument comprises a defined geometric dimension, and the processor generates a simulated representation of the therapeutic and/or diagnostic device and display the representation and position information on the model, or optionally overlay the representation and position information on the body image of the body lumen.

C72. The method of C68, wherein the body lumen location is selected by interacting with the display of the external body lumen image, or optionally interacting with the diagnostic and/or therapeutic system, or optionally interacting with at least one device via an interface.

C73. The method of C68, wherein 2D/3D model dimension information is calculated from the known spacing and dimensions of the plurality of imaging markers.

C4. The method of C69, wherein the model of the flexible elongate instrument is generated with a recaptured body image, and the location of the of the diagnostic and/or therapeutic device relative to the position of the flexible elongate instrument model is also generated displayed as an overlay with the recaptured external body image.

C75. The method of C74, wherein the recaptured body image is an X-ray angiogram which is obtained when the X-ray instrument is not emitting X-rays.

C76. The method of C68, further comprising recording at least one body lumen image of the flexible elongate instrument comprising a plurality of imaging markers inside the body lumen at the same orientation as the model, aligning the markers on the 2-dimensional model with the corresponding markers on at least one recorded image as a correlated unit, and superimposing on a display the body-lumen image with the aligned markers on the model.

C77. The method of C68, further comprising: storing at least one body lumen image of the flexible elongate instrument comprising a plurality of imaging markers inside the body lumen from any orientation to generate a recorded oriented body lumen image, aligning the markers from the 3-dimensional model with the markers on the recorded oriented body lumen image, and superimposing the oriented body lumen image with the model of the flexible elongate instrument comprising a plurality of imaging markers on a display.

C78. The method of any of C76 or C77, further comprising: storing a second body-lumen image obtained from the plurality of imaging markers within the body lumen, aligning the lumen positions of the two stored body lumen images, identifying differences in one or a plurality of selected imaging marker positions between the two stored body lumen images, measuring the differences in one or a plurality of selected imaging marker positions between the two stored body lumen images to obtain a self-correction coefficient, and optionally, applying the self-correction coefficient to a subsequent body lumen image obtained from the plurality of imaging markers within the body lumen.

C79. A computer configured to perform any of the methods of C34-C47 or C68-C78.

While example embodiments have been particularly shown and described, it will be understood by those skilled in the art that various changes in form and details may be made therein without departing from the scope of the embodiments encompassed by the appended claims.

What is claimed is:

1. A system for locating a medical device in a body lumen, comprising:
a first flexible elongate instrument comprising a plurality of imaging markers;
a location information sensor disposed at the first flexible elongate instrument or at a second flexible elongate instrument configured for relative movement with respect to the first flexible elongate instrument;
a processor configured to:
establish a reference coordinate system based on the plurality of imaging markers, the plurality of imaging markers being visible in a medical image comprising the first flexible elongate instrument disposed in a body lumen,
receive diagnostic scan or therapeutic delivery information at a plurality of locations of the body lumen from the first or second flexible elongate instrument, and
correlate the diagnostic scan or therapeutic delivery information with the imaging markers for the plurality of locations based on the reference coordinate system and location information as sensed by the location information sensor; and
a display configured to display a composite image comprising the correlated diagnostic scan or therapeutic delivery information and the imaging markers.

2. The system of claim 1, wherein the location information sensor is disposed on the first flexible elongate instrument.

3. The system of claim 2, wherein the location information sensor is a sensor configured to detect encoding markers of the second flexible elongate instrument.

4. The system of claim 2, wherein the first flexible elongate instrument is a guidewire and the second flexible elongate instrument comprises a diagnostic or therapeutic device.

5. The system of claim 4, wherein the second flexible elongate instrument comprises the diagnostic device, and the diagnostic device is an intravascular ultrasound (NUS) device, an optical coherence tomography (OCT) device, a fractional flow reserve (FFR) catheter, a photoacoustic device, an endoscopic device, an arthroscopic device, or a biopsy device.

6. The system of claim 4, wherein the second flexible elongate instrument comprises the therapeutic device, and the therapeutic device is an angioplasty device, an embolization device, a stent, an ablation device, a drug-delivery device, an optical delivery device, an atherectomy device, or an aspiration device.

7. The system of claim 3, wherein the second flexible elongate instrument comprises the encoding markers disposed at an inner circumferential surface of a catheter or liner configured for advancement over the first flexible elongate instrument.

8. The system of claim 1 wherein the location information sensor is disposed on the second flexible elongate instrument.

9. The system of claim 8, wherein the location information sensor is a sensor configured to detect encoding markers of the first flexible elongate instrument.

10. The system of claim 9, wherein the first flexible elongate instrument is a fractional flow reserve (FFR) wire.

11. The system of claim 1, wherein the location information sensor is a diagnostic sensor disposed on the second flexible elongate instrument.

12. The system of claim 11, wherein first flexible elongate instrument comprises a signal emitter configured to emit a signal for detection by the diagnostic sensor.

13. The system of claim 12, wherein the signal emitter is an ultrasound transducer, an optical light emitter, or a signal reflector configured to reflect a signal originating from the diagnostic sensor.

14. The system of claim 12, wherein correlating the diagnostic scan information with the imaging markers includes establishing a co-position location based on the detected signal.

15. The system of claim 1, wherein the first flexible elongate instrument is a diagnostic device and the location information sensor is a sensor that detects a push distance, a pullback distance, or a combination thereof of the diagnostic device.

16. The system of claim 15, wherein correlating the diagnostic scan information with the imaging markers includes establishing a start location of a diagnostic sensor of the diagnostic device based on a relative position of the diagnostic sensor to at least one of the plurality of imaging markers.

17. The system of claim 1, wherein the second flexible elongate instrument is a diagnostic device comprising at least one imaging marker and the location information sensor is a sensor that detects a push distance, a pullback distance, or a combination thereof of the diagnostic device.

18. The system of claim 17, wherein correlating the diagnostic scan information with the imaging markers includes establishing a start location of a diagnostic sensor of the diagnostic device based on a relative position of the at least one imaging marker of the diagnostic device and at least one of the plurality of imaging markers of the first flexible elongate instrument.

19. The system of claim 1, wherein the system further comprises the second flexible elongate instrument.

20. The system of claim 1, wherein the sensor is disposed at a distal portion of the first or second flexible elongate instrument.

21. The system of claim 1, wherein the processor is further configured to receive the medical image, and the reference coordinate system is two-dimensional.

22. The system of claim 1, wherein the processor is further configured to receive the medical image, the medical image including at least two medical images comprising the first flexible elongate instrument disposed in the body lumen, and wherein the reference coordinate system is three-dimensional.

23. The system of claim 1, wherein the location information sensor is a single element sensor.

24. A method for locating a medical device in a body lumen, comprising:
establishing a reference coordinate system based on a plurality of imaging markers of a first flexible elongate instrument disposed in a body lumen, the imaging markers visible in a medical image comprising the first flexible elongate instrument;
receiving diagnostic scan or therapeutic delivery information at a plurality of locations of the body lumen from the first flexible elongate instrument or a second flexible elongate instrument configured for relative movement with respect to the first flexible elongate instrument, at least one of the first and second flexible elongate instruments comprising a location information sensor;
correlating the diagnostic scan or therapeutic delivery information with the imaging markers for the plurality of locations based on the reference coordinate system and location information as sensed by the location information sensor; and
displaying a composite image comprising the correlated diagnostic scan or therapeutic delivery information and the imaging markers.

25. The method of claim 24, wherein the location information sensor is a sensor configured to detect encoding markers, and wherein the method further includes detecting encoding markings of one of the first and second flexible elongate instruments.

26. The method of claim 24, wherein the location information sensor is a diagnostic sensor disposed on the second flexible elongate instrument, and wherein the method further includes detecting a signal emitted by the first flexible elongate instrument.

27. The method of claim 26, wherein correlating the diagnostic scan information with the imaging markers includes establishing a co-position location based on the detected signal.

28. The method of claim 24, wherein the location information sensor is a sensor that detects a push distance, a pullback distance, or a combination thereof of the diagnostic device, one of the first and second flexible elongate instruments comprising the diagnostic device.

29. The method of claim 28, wherein correlating the diagnostic scan information with the imaging markers includes establishing a start location of a diagnostic sensor of the diagnostic device based on a relative position of the diagnostic sensor to at least one of the plurality of imaging markers.

30. The method of claim 28, wherein the second flexible elongate instrument is a diagnostic device comprising at least one imaging marker, and wherein correlating the diagnostic scan information with the medical image includes establishing a start location of a diagnostic sensor of the diagnostic device based on a relative position of at least one imaging marker of the diagnostic device and at least one of the plurality of imaging markers of the first flexible elongate instrument.

31.-64. (canceled)

65. The system of claim 1, wherein the medical image is an X-ray angiogram and the imaging markers are radiopaque imaging markers.

66. The method of claim 24, wherein the medical image is an X-ray angiogram and the imaging markers are radiopaque imaging markers.

67. The system of claim 1, further comprising a direction sensor configured to detect advancement and retraction of the relative movement of the first and second flexible elongate instruments.

68. The method of claim 24, further comprising receiving directional information from a direction sensor configured to detect advancement and retraction of the relative movement of the first and second flexible elongate instruments.

69. The system of claim 1, wherein the composite image further comprises a simulated representation of a treatment delivered to at least one of the plurality of locations.

70. The system of claim 1, wherein the composite image further comprises a simulated representation of a location of the diagnostic or therapeutic device with respect to the medical image.

71. The system of claim 70, wherein the simulated representation provides for a dimensional representation of the diagnostic or therapeutic device with respect to the lumen.

72. The method of claim 24, wherein displaying the composite image further includes displaying a simulated representation of a treatment delivered to at least one of the plurality of locations.

73. The method of claim 24, wherein displaying the composite image further includes displaying a simulated representation of a location of the diagnostic or therapeutic device with respect to the medical image.

74. The method of claim 73, wherein the simulated representation provides for a dimensional representation of the diagnostic or therapeutic device with respect to the lumen.

75.-77. (canceled)
